(19) United States
(12) Patent Application Publication
(10) Pub. No.: US 2023/0202973 A1
(43) Pub. Date: Jun. 29, 2023

(54) BARNESIN A, DERIVATIVES AND USES THEREOF

(71) Applicants: Leibniz-Institut Für Naturstoff-Forschung Und Infektionsbiologie E.V. (HKI), Jena (DE); Friedrich-Schiller-Universität Jena, Jena (DE)

(72) Inventors: Christine Beemelmanns, Jena (DE); Huijuan Guo, Jena (DE); Sandra Höfgen, Jena (DE); Maja Rischer, Jena (DE); Luka Raguz, Jena (DE); Francois Keiff, Jena (DE); Tobias Goris, Berlin (DE)

(21) Appl. No.: 16/978,447

(22) PCT Filed: Mar. 6, 2019
(86) PCT No.: PCT/EP2019/055500

§ 371 Date: Sep. 4, 2020

(30) Foreign Application Priority Data
Mar. 6, 2018 (EP) 18160274.9

(51) Int. Cl. C07C 279/14; C12N 15/52; C12P 13/02; C12N 9/00; C12N 9/10; C07C 237/52; C07C 279/12; A61P 33/02

(52) U.S. Cl. CPC C07C 279/14; C12N 15/52; C12P 13/02; C12N 9/93; C12N 9/1029; C07C 237/52; C07C 279/12; A61P 33/02

(57) ABSTRACT

This invention relates to a compound according to general formula (IA), which acts as a selective cysteine protease inhibitor; to a pharmaceutical composition containing one or more of the compound(s) of the invention; to a combination preparation containing at least one compound of the invention and at least one further active pharmaceutical ingredient; and to uses of said compound(s), including the use as a medicament.

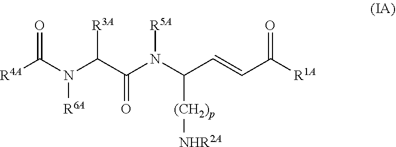

### FIELD OF THE INVENTION

This invention relates to a compound according to general formula (I), which acts as a selective cysteine protease inhibitor; to a pharmaceutical composition containing one or more of the compound(s) of the invention; to a combination preparation containing at least one compound of the invention and at least one further active pharmaceutical ingredient; and to uses of said compound(s), including the use as a medicament.

### BACKGROUND OF THE INVENTION

Natural products of microbial origin have been a rich source of compounds for drug discovery. In particular, actinomycetes and myxobacteria are prime producers of industrially important natural products. However, studies of such well-investigated organisms have led to high rediscovery rate of already known natural product classes. Yet, microbial natural products are still one of the most promising sources for novel drugs. This is, because natural products own an element of structural complexity which allows for the specific and effective inhibition of many targets (e.g. proteins, DNA, RNA). For instance, nonribosomally synthesized peptides and polyketides are classes of secondary metabolites in bacteria, fungi, and plants that exhibit a wide range of bioactivities which are interesting for pharmaceutical applications.

Global genome mining efforts implied the general abundance of secondary metabolite gene clusters within all bacterial phyla (Ziemert and Jensen, Methods in enzymology 2012; 517:161). The increasing number of known genome sequences in combination with bioinformatic tools allows identifying so far uncharacterized and interesting putative biosynthetic gene clusters in microorganisms that may encode novel bioactive secondary metabolites.

Epsilonproteobacteria are among those that have so far been neglected in the characterization of unprecedented secondary metabolites and biosynthetic pathways. Bacteria belonging to the genus Sulfurospirillum (Epsilonproteobacteria) are frequently found in sediments, aquatic habitats (fresh and salt water), oil reservoirs, and sewage plants, and include the non-dehalogenating species Sulfurospirillum barnesii (Goris and Dieckert, The genus Sulfurospirillum. In Organohalide-respiring bacteria. Adrian, L., and Löffler, F. E. (eds). Berlin Heidelberg: Springer 2016, Goris et al, Environmental microbiology 2014; 16(11): 3562-3580).

Cysteine proteases are validated targets for treatment of a number of diseases, including neurodegenerative disorders, e.g. Alzheimer's disease, (Siklos et al., Acta Pharmaceutica Sinica B 2015; 5(6): 506-519); parasitic infections, e.g. Chagas disease and human African trypanosomiasis, (Ferreira and Andricopulo, Pharmacology & Therapeutics 2017; 180: 49-61); and invasive and metastatic cancers (Mason and Joyce, Trends in Cell Biology 2011, 21(4): 228-237).

Although much progress has been made in the development of antitumor agents, cancer is still one of the leading causes of death. In addition, many of the current therapies are associated with severe side effects. Similar considerations apply to Chagas disease and human African trypanosomiasis, which are endemic life-threatening conditions in Latin America and Africa for the treatment of which only a few drugs with serious efficacy and safety drawbacks are available (Ferreira and Andricopulo, loc. cit.). Available drug therapies for neurodegenerative disorders are also dismal as many suffer from serious efficacy and toxicity problems.

In view of the deficits of the prior art and the severe conditions associated with a pathophysiological level of cysteine proteases, both acute and chronic, there is a need for novel cysteine protease inhibitors.

### Summary and Description of the Invention

The present invention was made in view of the prior art and the needs described above, and, therefore, the object of the present invention is to provide novel cysteine protease inhibitors according to general formula (I). Other objects of the present invention are to provide a pharmaceutical composition comprising at least one cysteine protease inhibitor as described herein; a combination preparation containing at least one compound of the invention and at least one further active pharmaceutical ingredient; and uses of the compound(s) of the invention, including the use as a medicament.

These objects are solved by the subject-matter of the attached claims as will become apparent upon reference to the following description and definitions. In particular, the inventors established that the compound according to the invention is a potent selective inhibitor of cysteine proteases.

These surprising and unexpected results allow an alternative therapeutic, preventive and/or curative role to be conceived for the compound according to the invention in the prevention and/or treatment of various conditions or disorders associated with a pathophysiological level of a cysteine protease, including neurodegenerative disorders, e.g. Alzheimer's disease; parasitic infections, e.g. Chagas disease and human African trypanosomiasis; and invasive and metastatic cancers.

Accordingly, the present invention is directed to a compound of the general formula (I):

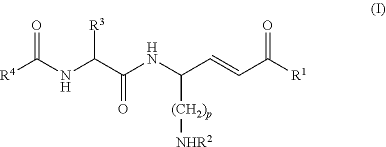

or a pharmacologically acceptable salt thereof, wherein
R¹ represents a hydrogen atom, —OR¹¹, —NR¹¹R¹²; or a (C₁-C₆)alkyl, (C₂-C₆)alkenyl, (C₂-C₆) alkinyl, or (C₃-C₆) cycloalkyl group, all of which groups may optionally be substituted;

R¹¹ and R¹² each, independently of one another, represents a hydrogen atom, or a (C₁-C₆)alkyl, (C₂-C₆)alkenyl, (C₂-C₆) alkinyl or (C₁-C₆)heteroalkyl group, all of which groups may optionally be substituted, or R¹¹ and R¹² together with the nitrogen atom to which they are attached form a 5- to 8-membered heterocyclic or heteroaromatic ring that can be substituted with from 0 to 3 substituents which substituents are each independently selected from halogen atom, —OH, —NH₂, —NHC₁₋₆ alkyl, and —N(C₁₋₆ alkyl)₂;

R² is a hydrogen atom, a group of formula —C(═NH)NH₂, or a group of formula —C(═O)R²¹;

R²¹ represents a (C₁-C₆)alkyl, (C₂-C₆)alkenyl, (C₂-C₆) alkinyl or (C₁-C₆)heteroalkyl group; all of which groups may optionally be substituted;

R³ is an amino acid side chain; a hydrogen atom, a halogen atom, OH; or an alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl group; all of which groups may optionally be substituted;

R⁴ is an alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl group; all of which groups may optionally be substituted; and

n is an integer of from 1 to 6.

The present invention is further directed to a compound of the general formula (IA):

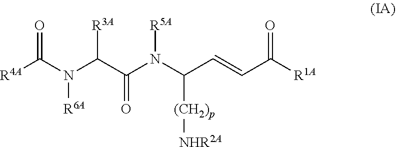

or a pharmacologically acceptable salt thereof, wherein
R^{1A}, R^{2A}, R^{3A} and R^{4A} are defined as the corresponding substituents R¹, R², R³ and R⁴ in general formula (I) above; p is defined as n in general formula (I) above; and

R^{5A} and R^{6A} each, independently of one another, represents a hydrogen atom or a methyl group.

Compounds are usually described herein using standard nomenclature or the definitions presented below. For compounds having asymmetric centers, it should be understood that (unless otherwise specified) all of the optical isomers and mixtures thereof are encompassed. Compounds with two or more asymmetric elements can also be present as mixtures of diastereomers. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope (i.e., an atom having the same atomic number but a different mass number). By way of general example, and without limitation, isotopes of hydrogen include tritium and deuterium and isotopes of carbon include ¹¹C, ¹³C, and ¹⁴C.

Compounds according to the formulas provided herein, which have one or more stereogenic center(s), have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors or by resolution of the racemates. Resolution of the racemates can be accomplished, for example, by conventional methods such as crystallization in the presence of a resolving agent, or chromatography, using, for example a chiral HPLC column.

The compound according to the invention is described herein using a general formula that includes variables such as, e.g. R¹, R², R³, and R⁴. Unless otherwise specified, each variable within such a formula is defined independently of any other variable, and any variable that occurs more than one time in a formula is defined independently at each occurrence. Thus, for example, if a group is shown to be substituted with 0-2 R*, the group may be unsubstituted, or substituted with 1 or 2 group(s) R*, wherein R* at each occurrence is selected independently from the corresponding definition of R*. Also, it should be understood that combinations of substituents and/or variables are permissible only if such combinations result in stable compounds, i.e., compounds that can be isolated, characterized and tested for biological activity.

As used herein a wording defining the limits of a range of length such as, e. g., “from 1 to 5” means any integer from 1 to 5, i. e. 1, 2, 3, 4 and 5. In other words, any range defined by two integers explicitly mentioned is meant to comprise and disclose any integer defining said limits and any integer comprised in said range. For example, the term “C₁-C₆” refers to 1 to 6, i.e. 1, 2, 3, 4, 5 or 6, carbon atoms. Further, the prefix “(Cx-y)” as used herein means that the chain, ring or combination of chain and ring structure as a whole, indicated in direct association of the prefix, may consist of a minimum of x and a maximum of y carbon atoms (i.e. x<y), wherein x and y represent integers defining the limits of the length of the chain (number of carbon atoms) and/or the size of the ring (number of carbon ring atoms).

A “synthetic compound” or “a not naturally occurring derivative” of a compound disclosed herein is a compound that is chemically distinct from the natural compound, e.g. different stereochemistry, modified by a substituent, etc.

A “pharmacologically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. Such pharmaceutical salts include mineral and organic acid salts of basic residues such as amines, as well as alkali or organic salts of acidic residues such as carboxylic acids.

Suitable pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fiumaric, maleic, propionic, hydroxymaleic, hydroiodic, phenylacetic, alkanoic such as acetic, HOOC—(CH₂)ₙ—COOH where n is any integer from 0 to 4 (i.e., 0, 1, 2, 3, or 4) and the like. Similarly, pharmaceutically acceptable cations include, but are not limited to sodium, potassium, calcium, aluminum, lithium and ammonium. Those of ordinary skill in the art will recognize further pharmacologically acceptable salts for the compounds provided herein. In general, a pharmacologically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of nonaqueous media, such as ether, ethyl acetate, ethanol, isopropanol or acetonitrile, is preferred.

It will be apparent that each compound of formula (I) may, but need not, be present as a hydrate, solvate or non-covalent complex. In addition, the various crystal forms and polymorphs are within the scope of the present invention. The hydratization/hydration may occur during the process of production or as a consequence of the hygroscopic nature of the initially water free compounds. The solvates and/or hydrates may e.g. be present in solid or liquid form.

A “substituent,” as used herein, refers to a molecular moiety that is covalently bonded to an atom within a molecule of interest. For example, a substituent on a ring may be a moiety such as a halogen atom, an alkyl, haloalkyl, hydroxy, cyano, or amino group, or any other substituent described herein that is covalently bonded to an atom, preferably a carbon or nitrogen atom, that is a ring member. The term “substituted,” as used herein, means that any one or more hydrogen atom(s) on the designated atom or group (e.g. alkyl, alkoxy, alkoxyalkyl, cycloalkyl, heterocycloalkyl, heteroaryl) is replaced with a selection from the indicated substituents, provided that the designated atom's normal valence or the group's number of possible sites for substitution is not exceeded, and that the substitution results in a stable compound, i.e. a compound that can be isolated, characterized and tested for biological activity. When a substituent is oxo, i.e., ═O, then 2 hydrogens on the atom are replaced. An oxo group that is a substituent of an aromatic carbon atom results in a conversion of —CH— to —C(═O)— and may lead to a loss of aromaticity. For example, a pyridyl group substituted by oxo is a pyridone. The indication mono-, di-, tri or tetrasubstituted denotes groups having one (mono), two (di), three (tri) or four substituents, provided that the substitution does not exceeded the number of possible sites for substitution and results in a stable compound. For example, a monosubstituted imidazolyl group may be an (imidazolidin-2-on)yl group and a disubstituted isoxazolyl group may be a ((3,5-dimethyl)isoxazolyl) group.

As used herein, “comprising”, “including”, “containing”, “characterized by”, and grammatical equivalents thereof are inclusive or open-ended terms that do not exclude additional, unrecited elements or method steps. Yet, “comprising”, etc. is also to be interpreted as including the more restrictive terms “consisting essentially of” and “consisting of”, respectively.

As used herein, “consisting of” excludes any element, step, or ingredient not specified in the claim.

When trade names are used herein, it is intended to independently include the trade name product formulation, the generic drug, and the active pharmaceutical ingredient(s) of the trade name product.

In general, unless defined otherwise, technical and scientific terms used herein have the same meaning as commonly understood by one of ordinary skill in the art to which this invention belongs, and are consistent with general textbooks and dictionaries.

As used herein, the term amino acid encompasses both “proteinogenic” amino acids and “non-conventional” amino acids. “Proteinogenic amino acids” denote α-amino derivatives of aliphatic carboxylic acids that are naturally incorporated into polypeptides, i.e. the twenty two amino acids, which are selected from the group consisting of Glycine, Leucine, Isoleucine, Valine, Alanine, Phenylalanine, Tyrosine, Tryptophan, Aspartic acid, Asparagine, Glutamic acid, Glutamine, Cysteine, Methionine, Arginine, Lysine, Proline, Serine, Threonine, Histidine, Selenocysteine and Pyrrolysine; and all stereomeric isoforms, i.e. D, L-, D- and L-amino acids thereof, are encompassed. The term “non-conventional amino acid” refers to unnatural amino acids or chemical amino acid analogues, e.g. α,α-disubstituted amino acids, N-alkyl amino acids, homo-amino acids, dehydroamino acids, aromatic amino acids (other than phenylalanine, tyrosine and tryptophan), and ortho-, meta- or para-aminobenzoic acid. Non-conventional amino acids also include compounds which have an amine and carboxyl functional group separated in a 1,3 or larger substitution pattern, such as β-alanine, γ-amino butyric acid, Freidinger lactam, the bicyclic dipeptide (BTD), amino-methyl benzoic acid and others well known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art may also be used. The use of analogues or non-conventional amino acids may improve the stability and biological half-life of the added peptide since they are more resistant to breakdown under physiological conditions. The person skilled in the art will be aware of similar types of substitution which may be made. A non limiting list ofnon-conventional amino acids which may be used as suitable building blocks for a peptide and their standard abbreviations (in brackets) is as follows: α-aminobutyric acid (Abu), L-N-methylalanine (Nmala), α-amino-α-methylbutyrate (Mgabu), L-N-methylarginine (Nmarg), aminocyclopropane (Cpro), L-N-methylasparagine (Nmasn), carboxylate L-N-methylaspartic acid (Nmasp), aniinoisobutyric acid (Aib), L-N-methylcysteine (Nmcys), aminonorbornyl (Norb), L-N-methylglutamine (Nmgln), carboxylate L-N-methylglutamic acid (Nmglu), cyclohexylalanine (Chexa), L-N-methylhistidine (Nmhis), cyclopentylalanine (Cpen), L-N-methylisolleucine (Nmile), L-N-methylleucine (Nmleu), L-N-methyllysine (Nmlys), L-N-methylmethionine (Nmmet), L-N-methylnorleucine (Nmnle), L-N-methylnorvaline (Nmnva), L-N-methylomithine (Nmom), L-N-methylphenylalanine (Nmphe), L-N-methylproline (Nmpro), L-N-methylserine (Nmser), L-N-methylthreonine (Nmthr), L-N-methyltryptophan (Nmtrp), D-omithine (Dom), L-N-methyltyrosine (Nmtyr), L-N-methylvaline (Nmval), L-N-methylethylglycine (Nmetg), L-N-methyl-t-butylglycine (Nmtbug), L-norleucine (NIe), L-norvaline (Nva), α-methyl-aminoisobutyrate (Maib), α-methyl-γ-aminobutyrate (Mgabu), D-α-methylalanine (Dmala), α-methylcyclohexylalanine (Mchexa), D-α-methylarginine (Dmarg), α-methylcylcopentylalanine (Mcpen), D-α-methylasparagine (Dmasn), α-methyl-α-napthylalanine (Manap), D-α-methylaspartate (Dmasp), α-methylpenicillamine (Mpen), D-α-methylcysteine (Dmcys), N-(4-aminobutyl)glycine (NgIu), D-α-methylglutamine (Dmgln), N-(2-aminoethyl)glycine (Naeg), D-α-methylhistidine (Dmhis), N-(3-aminopropyl)glycine (Nom), D-α-methylisoleucine (Dmile), N-amino-α-methylbutyrate (Nmaabu), D-α-methylleucine (Dmleu), α-napthylalanine (Anap), D-α-methyllysine (Dmlys), N-benzylglycine (Nphe), D-α-methylmethionine (Dmmet), N-(2-carbamylethyl)glycine (NgIn), D-α-methylornithine (Dmom), N-(carbamylmethyl)glycine (Nasn), D-α-methylphenylalanine (Dmphe), N-(2-carboxyethyl)glycine (NgIu), D-α-methylproline (Dmpro), N-(carboxymethyl)glycine (Nasp), D-α-methylserine (Dmser), N-cyclobutylglycine (Ncbut), D-α-methylthreonine (Dmthr), N-cycloheptylglycine (Nchep), D-α-methyltryptophan (Dmtrp), N-cyclohexylglycine (Nchex), D-α-methyltyrosine (Dmty), N-cyclodecylglycine (Ncdec), D-α-methylvaline (Dmval), N-cylcododecylglycine (Ncdod), D-N-methylalanine (Dnmala), N-cyclooctylglycine (Ncoct), D-N-methylarginine (Dnmarg), N-cyclopropylglycine (Ncpro), D-N-methylasparagine (Dnmasn), N-cycloundecylglycine (Ncund), D-N-methylaspartate (Dnmasp), N-(2,2-diphenylethyl)glycine (Nbhm), D-N-methylcysteine (Dnmcys), N-(3,3-diphenylpropyl)glycine (Nbhe), D-N-methylglutamine (Dnmgln), N-(3-guanidinopropyl)glycine (Narg), D-N-methylglutamate (Dnmglu), N-(1-hydroxyethyl)glycine (Ntbx), D-N-methylhistidine (Dnmhis), N-(hydroxyethyl))glycine (Nser), D-N-methylisoleucine (Dnmile), N-(imidazolylethyl))glycine (Nhis), D-N-methylleucine (Dnmleu), N-(3-indolylyethyl)glycine (Nhtrp), D-N-methyllysine (Dnnilys), N-methyl-γ-aminobutyrate (Nmgabu), N-methylcyclohexylalanine (Nmchexa), D-N-methylmethionine (Dnmmet), D-N-methylomithine (Dnmom), N-methylcyclopentylalanine (Nmcpen), N-methylglycine (NaIa), D-N-methylphenylalanine (Dnmphe), N-methylaminoisobutyrate (Nmaib), D-N-methylproline (Dnmpro), N-(1-methylpropyl)glycine (Nile), D-N-methylserine (Dnmser), N-(2-methylpropyl)glycine (Nleu), D-N-methylthreonine (Dnmthr), D-N-methyltryptophan (Dnmtrp), N-(1-methylethyl)glycine (Nval), D-N-methyltyrosine (Dnmtyr), N-methyla-napthylalanine (Nmanap), D-N-methylvaline (Dnmval), N-methylpenicillamine (Nmpen), γ-aminobutyric acid (Gabu), N-(p-hydroxyphenyl)glycine (Nhtyr), L-/-butylglycine (Tbug), N-(thiomethyl)glycine (Ncys), L-ethylglycine (Etg), penicillamine (Pen), L-homophenylalanine (Hphe), L-α-methylalanine (Mala), L-α-methylarginine (Marg), L-α-methylasparagine (Masn), L-α-methylaspartate (Masp), L-α-methyl-t-butylglycine (Mtbug), L-α-methylcysteine (Mcys), L-methylethylglycine (Metg), L-α-methylglutamine (MgIn), L-α-methylglutamate (MgIu), L-α-methylhistidine (Mhis), L-α-methylhomophenylalanine (Mhphe), L-α-methylisoleucine (Mile), N-(2-methylthioethyl)glycine (Nmet), L-α-methylleucine (Mleu), L-α-methyllysine (Mlys), L-α-methylmethionine (Mmet), L-α-methylnorleucine (MnIe), L-α-methylnorvaline (Mnva), L-α-methylornithine (Morn), L-α-methylphenylalanine (Mphe), L-α-methylproline (Mpro), L-α-methylserine (Mser), L-α-methylthreonine (Mthr), L-α-methyltryptophan (Mtrp), L-α-methyltyrosine (Mtyr), L-α-methylvaline (Mval), L-N-methylhomophenylalanine (Nmhphe), N—(N-(2,2-diphenylethyl)carbamylmethyl)glycine (Nnbhm), N—(N-(3,3-diphenylpropyl)carbamylmethyl)glycine (Nnbhe), 1-carboxy-1-(2,2-diphenyl-ethylamino)cyclopropane (Nmbc), L-O-methyl serine (Omser), L-O-methyl homoserine (Omhser).

The expression alkyl or alkyl group denotes a saturated, straight-chain or branched hydrocarbon group that contains from 1 to 20 carbon atoms, preferably from 1 to 12 carbon atoms, more preferably from 1 to 6 carbon atoms, or the number of carbon atoms indicated in the prefix. If an alkyl is substituted, the substitution may take place, independently of one another, by mono-, di-, or tri-substitution of individual carbon atoms of the molecule, e.g. 1, 2, 3, 4, 5, 6, or 7 hydrogen atom(s) may, at each occasion independently, be replaced by a selection from the indicated substituents. The foregoing also applies if the alkyl group forms a part of a group, e.g. haloalkyl, hydroxyalkyl, alkylamino, alkoxy, or alkoxyalkyl. Examples of an alkyl group include methyl, ethyl, propyl, iso-propyl, n-butyl, iso-butyl, sec-butyl, tert-butyl, n-pentyl, iso-pentyl, n-hexyl, 2,2-dimethylbutyl, or n-octyl, and examples of a substituted alkyl group or a group where the alkyl forms a part of a group, include haloalkyl, e.g. a trifluoromethyl or a difluoromethyl group; hydroxyalkyl, e.g. hydroxymethyl or 2-hydroxyethyl group, and a methoxymethyl group. The term “(C₁₋₆) alkyl” includes, for example, H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)—, H₃C—CH₂—CH(CH₂CH₃)—, —CH₂CH₂CH₂CH₂CH₂CH₃, —CH(CH₃)CH₂CH₂CH₂CH₃, (H₃CH₂C)CH(CH₂CH₂CH₃)—, —C(CH₃)₂(CH₂CH₂CH₃), —CH(CH₃)CH(CH₃)CH₂CH₃, and —CH(CH₃)CH₂CH(CH₃)₂.

The expression alkoxy or alkoxy group refers to an alkyl group singular bonded to oxygen, i.e. —O— alkyl. The term “(C₁-C₆) alkoxy” includes, for example, methoxy, ethoxy, n-propoxy, isopropoxy, n-butoxy, sec-butoxy, isobutoxy, tert-butoxy, n-pentyloxy, tert-amyloxy- or n-hexyloxy, and accordingly (C₁-C₃)alkoxy includes methoxy, ethoxy, n-propoxy, or isopropoxy.

The expression alkoxyalkyl or alkoxyalkyl group refers to an alkyl group singular bonded to one or more alkoxy group(s), e.g. -alkyl-O-alkyl or -alkyl-O-alkyl-O-alkyl. The term “(C₂-C₅) alkoxyalkyl” includes, for example, methoxymethyl, methoxyethoxymethyl, and 1-ethoxyethyl.

The expression haloalkyl or haloalkyl group refers to an alkyl group in which one, two, three or more hydrogen atoms have been replaced independently of each other by a halogen atom. The term “(C₁-C₃) haloalkyl” includes, for example, fluoromethyl, difluoromethyl, trifluoromethyl, chloromethyl, dichloromethyl, bromomethyl, dibromomethyl, iodomethyl, (1- or 2-)haloethyl (e.g. (1- or 2-)fluoroethyl or (1- or 2-)chloroethyl), (2- or 3-) halopropyl (e.g. (2- or 3-) fluoropropyl or (2- or 3-) chloropropyl). The expression hydroxyalkyl or hydroxyalkyl group refers to an alkyl group in which one, two, three or more hydrogen atoms have been replaced independently of each other by a hydroxy (OH) group. The term “(C₁-C₄) hydroxyalkyl” includes, for example, hydroxymethyl, hydroxyethyl, hydroxypropyl and hydroxybutyl.

The expression alkenyl or alkenyl group refers to an at least partially unsaturated, straight-chain or branched hydrocarbon group that contains one or more double bond(s) and from 2 to 20 carbon atoms, preferably from 2 to 12 carbon atoms, more preferably from 2 to 6 carbon atoms, wherein said alkenyl group may be optionally substituted. Examples of an unsubstituted alkenyl group include an ethenyl (vinyl), propenyl (allyl), iso-propenyl, butenyl, isoprenyl and hex-2-enyl group. Preferably, an alkenyl group has one or two, especially one, double bond(s).

The expression alkynyl or alkynyl group refers to an at least partially unsaturated, straight-chain or branched hydrocarbon group that contains one or more triple bond(s) and from 2 to 20 carbon atoms, preferably from 2 to 12 carbon atoms, more preferably from 2 to 6, e.g. 2, 3 or 4, carbon atoms, wherein said alkynyl group may be optionally substituted. Examples of an unsubstituted alkynyl group include an ethynyl (acetylenyl), propynyl, butynyl or propargyl group. Preferably, an alkynyl group has one or two, especially one, triple bond(s).

As used herein, the expression heteroalkyl or heteroalkyl group refers to an alkyl, alkenyl or alkynyl group (straight chain or branched) as defined above, in which one or more, preferably 1 to 8, and more preferably 1, 2, 3 or 4, carbon atom(s) has/have been replaced (each independently of the others) by an oxygen, nitrogen, phosphorus, boron, selenium, silicon or sulphur atom, preferably by an oxygen, sulphur or nitrogen atom, or by a SO or SO₂ group, wherein said heteroalkyl group may be optionally substituted.

As a result, the expression heteroalkyl encompasses groups containing 1 to 19 carbon atoms, preferably from 1 to 11 carbon atoms, more preferably from 1 to 5, i.e. 1, 2, 3, 4 or 5, carbon atoms, and accordingly may also be referred to as C₁-C₁₉, C₁-C₁₁, and C₁-C₅ heteroalkyl, respectively. Preferably, a C₁-C₅ heteroalkyl group contains from 1 to 5, e.g. 1, 2, 3 or 4, carbon atoms and 1, 2, 3 or 4, preferably 1, 2 or 3, heteroatoms selected from oxygen, nitrogen and sulphur (especially oxygen and nitrogen). Especially preferably, the expression heteroalkyl refers to an alkyl group as defined above (straight-chain or branched) in which one or more, preferably 1 to 6, especially preferably 1, 2, 3 or 4, carbon atoms have been replaced by an oxygen, sulfur or nitrogen atom. Examples of heteroalkyl groups are alkylamino, dialkylamino, alkylaminoalkyl, dialkylaminoalkyl, alkylnitrile, acyl, acylalkyl, alkoxycarbonyl, acyloxy, acyloxyalkyl, carboxyalkylamide, alkoxycarbonyloxy, alkylcarbamoyl, alkylamido, alkylcarbamoylalkyl, alkylamidoalkyl, alkylcarbamoyloxyalkyl, alkylureidoalkyl, alkoxy, alkoxyalkyl, or alkylthio group; all of which groups may be optionally substituted.

Examples of heteroalkyl groups include, for example, groups of formulae: R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})-Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{d} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a direct bond, a C₁-C₆ alkylene, a C₂-C₆ alkenylene or a C₂-C₆ alkynylene group. Specific examples of heteroalkyl groups, which may optionally be substituted, include acyl, methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, isopropylethylamino, methylaminomethyl, ethylaminomethyl, diisopropylaminoethyl, dimethylaminomethyl, dimethylaminoethyl, acetyl, propionyl, butyryloxy, acetyloxy, methoxycarbonyl, ethoxycarbonyl, isobutyrylamino-methyl, N-ethyl-N-methylcarbamoyl, N-methylcarbamoyl, cyano, nitrile, isonitrile, thiocyanate, isocyanate, isothiocyanate and alkylnitrile.

The expression cycloalkyl or cycloalkyl group refers to a saturated or partially unsaturated cyclic group that contains one or more rings (preferably 1 or 2), containing from 3 to 14 ring carbon atoms, preferably from 3 to 10 (more preferably 3, 4, 5, 6 or 7) ring carbon atoms, wherein the cycloalkyl group may be optionally substituted. In an embodiment a partially unsaturated cyclic group has one, two or more double bonds, such as a cycloalkenyl group. Specific examples of an unsubstituted cycloalkyl group are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, cyclopentylcyclohexyl, and a cyclohex-2-enyl group.

The expression heterocycloalkyl or heterocycloalkyl group refers to a cycloalkyl group as defined above in which one or more, preferably 1, 2 or 3, ring carbon atoms have been replaced each independently of the others by an oxygen, nitrogen, or sulphur atom (preferably oxygen or nitrogen), or by a SO or SO2 group. A heterocycloalkyl group has preferably 1 or 2 ring(s) containing from 3 to 10 (more preferably 3, 4, 5, 6 or 7, and most preferably 5, 6 or 7) ring atoms. Examples are an aziridinyl, oxiranyl, thiiranyl, oxaziridinyl, dioxiranyl, azetidinyl, oxetanyl, thietanyl, diazetidinyl, dioxetanyl, dithietanyl, pyrrolidinyl, tetrahydrofuranyl, thiolanyl, phospholanyl, silolanyl, azolyl, thiazolyl, isothiazolyl, imidazolidinyl, pyrazolidinyl, oxazolidinyl, isoxazolidinyl, thiazolidinyl, isothiazolidinyl, dioxolanyl, dithiolanyl, piperazinyl, morpholinyl, thiomorpholinyl, trioxanyl, azepanyl, oxepanyl, thiepanyl, homopiperazinyl, or urotropinyl group. Further examples are a 2-pyrazolinyl group, and also a lactam, a lactone and a cyclic imide. The heterocycloalkyl group can be optionally substituted, and may be saturated or mono-, di- or tri-unsaturated. As a result, the expression heterocycloalkyl group also encompasses a group derived from a carbohydrate or saccharide, such as furanoses or pentoses, e.g. arabinose, ribose, xylose, lyxose or desoxyribose, or pyranoses/hexoses or derivatives thereof, e.g. allose, altrose, glucose, mannose, gulose, idose, galactose, talose, 6-carboxy-D-glucose, 6-carboxy-D-galactose, N-acetylchitosamine, glucosamine, N-acetylchondrosamin, fucose, rhamnose, or chinovose.

The expression alkylcycloalkyl or alkylcycloalkyl group refers to a group containing both cycloalkyl and also an alkyl, alkenyl or alkynyl group in accordance with the above definitions, for example alkyl-cycloalkyl, cycloalkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (preferably 3, 4, 5, 6 or 7) carbon atoms, and one or two alkyl, alkenyl or alkynyl groups having 1 or 2 to 6 carbon atoms, the cyclic groups being optionally substituted.

The expression heteroalkylcycloalkyl or heteroalkylcycloalkyl group refers to alkylcycloalkyl groups as defined above in which one or more (preferably 1, 2 or 3) carbon atoms have been replaced each independently of the others by an oxygen, nitrogen, silicon, selenium, phosphorus or sulphur atom (preferably oxygen, sulphur or nitrogen). A heteroalkylcycloalkyl group preferably contains 1 or 2 ring systems having from 3 to 10 (preferably 3, 4, 5, 6 or 7) ring atoms, and one or two alkyl, alkenyl, alkynyl or heteroalkyl groups having from 1 or 2 to 6 carbon atoms. Examples of such groups are alkylhetero-cycloalkyl, alkylheterocycloalkenyl, alkenylheterocycloalkyl, alkynylheterocycloalkyl, hetero-alkylcycloalkyl, heteroalkylheterocycloalkyl and heteroalkylheterocycloalkenyl, the cyclic groups being optionally substituted and saturated or mono-, di- or tri-unsaturated.

The expressions aryl, Ar or aryl group refer to an aromatic group that contains one or more rings containing from 6 to 14 ring carbon atoms (C₆-C₁₄), preferably from 6 to 10 (C₆-C₁₀), more preferably 6 ring carbon atoms, wherein the aryl group may be optionally substituted. Examples of an unsubstituted aryl group include a phenyl, naphthyl, biphenyl, or indanyl group.

The expression heteroaryl or heteroaryl group refers to an aromatic group that contains one or more rings containing from 5 to 14 ring atoms, preferably from 5 to 10 (more preferably 5 or 6) ring atoms, and contains one or more (preferably 1, 2, 3 or 4) oxygen, nitrogen, phosphorus or sulphur ring atoms (preferably 0, S or N), wherein the heteroaryl group may be optionally substituted. Examples of an unsubstituted heteroaryl group include 2-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl.

The expression aralkyl or aralkyl group refers to a group containing both, aryl and also alkyl, alkenyl, alkynyl and/or cycloalkyl groups in accordance with the above definitions, wherein the aralkyl group may be optionally substituted. As a result, the expression aralkyl group encompasses groups such as, for example, arylalkyl, arylalkenyl, arylalkynyl, arylcycloalkyl, arylcycloalkenyl, alkylarylcycloalkyl and alkylarylcycloalkenyl groups, wherein all of said groups may be optionally substituted. Specific examples of an unsubstituted aralkyl group include toluene, xylene, mesitylene, styrene, benzyl chloride, o-fluorotoluene, 1H-indene, tetralin, dihydronaphthalene, indanone, phenylcyclopentyl, cumene, cyclo-hexylphenyl, fluorene and indan. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl or heteroaralkyl group refers to an aralkyl group as defined above in which one or more (preferably 1, 2, 3 or 4) carbon atoms have been replaced each independently of the others by an oxygen, nitrogen, silicon, selenium, phosphorus, boron or sulphur atom (preferably oxygen, sulphur or nitrogen), that is to say to groups containing both aryl or heteroaryl and also alkyl, alkenyl, alkynyl and/or heteroalkyl and/or cycloalkyl and/or heterocycloalkyl groups in accordance with the above definitions, wherein the heteroaralkyl group may be optionally substituted. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced each independently of the others by oxygen, sulphur or nitrogen atoms. Examples of heteroaralkyl groups are arylheteroalkyl, arylheterocycloalkyl, arylheterocycloalkenyl, arylalkylheterocycloalkyl, arylalkenylheterocycloalkyl, arylalkynylheterocycloalkyl, arylalkylhetero-cycloalkenyl, heteroarylalkyl, heteroarylalkenyl, heteroarylalkynyl, heteroarylheteroalkyl, heteroaryl-cycloalkyl, heteroarylcycloalkenyl, heteroarylheterocycloalkyl, heteroarylheterocycloalkenyl, hetero-arylalkylcycloalkyl, heteroarylalkylheterocycloalkenyl, heteroarylheteroalkylcycloalkyl, heteroaryl-heteroalkylcycloalkenyl, heteroalkylheteroarylalkyl and heteroarylheteroalkylheterocycloalkyl groups; all of which groups may be optionally substituted and the cyclic moieties of said groups being saturated or mono-, di- or tri-unsaturated. Specific examples of a heteroaralkyl group include a tetrahydroisoqui-nolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

The expression halogen or halogen atom as preferably used herein means fluorine, chlorine, bromine, or iodine.

The expression “optionally substituted” refers to groups in which one or more hydrogen atoms, e.g. 1 to 7 hydrogen atoms, preferably 1 to 5 hydrogen atoms, and more preferably 1 to 3 hydrogen atoms, have been replaced each independently of the others by fluorine, or chlorine atoms or by OH, ═O, SH, ═S, NH₂, ═NH, CN or NO₂ groups. The expression “optionally substituted” refers furthermore to groups in which one or more hydrogen atoms, e.g. 1 to 7 hydrogen atoms, preferably 1 to 5 hydrogen atoms, and more preferably 1 to 3 hydrogen atoms, have been replaced each independently of the others by unsubstituted C₁-C₆alkyl, (C₁-C₆) haloalkyl (e.g. a fluoromethyl, trifluoromethyl, chloromethyl, (1- or 2-) haloethyl (e.g. (1- or 2-) chloroethyl), or (2- or 3-) halopropyl (e.g. (2- or 3-) fluoropropyl) group), (C₁-C₆) hydroxyalkyl (e.g. a hydroxymethyl, (1- or 2-)hydroxyethyl, or (2- or 3-) hydroxypropyl group), unsubstituted C₂-C₆alkenyl, unsubstituted C₂-C₆alkynyl, unsubstituted C₁-C₆heteroalkyl (e.g. C₁-C₆alkoxy; or C₁-C₆alkoxyalkyl), unsubstituted C₃-C₇cycloalkyl, unsubstituted C₂-C₇heterocycloalkyl, unsubstituted C₆-C₁₀aryl, unsubstituted C₁-C₈heteroaryl, unsubstituted C₇-C₁₂aralkyl or unsubstituted C₂-C₁₁heteroaralkyl groups.

Generally preferred substituents include: halogen atoms (e.g. F, Cl, Br), groups of formula —OH, —O—C₁₋₆ alkyl (e.g. —OMe, —OEt, —O-nPr, —O-iPr, —O-nBu, —O-iBu or —O-tBu), —NH₂, —NHC₁₋₆ alkyl, —N(C₁₋₆ alkyl)₂, —COOH, —SO₃H, ═O, —SO₂NH₂, —CONH₂, —CN, —C₁₋₆ alkyl (e.g. -Me, -Et, -nPr, -iPr, -nBu, -iBu, -tBu), —CF₃, —SH, —S—C₁₋₆ alkyl, NHAc, —NO₂, —NHCONH₂, —SO₂Me, and cyclopropyl.

The expression hydrocarbon group refers to groups consisting of carbon and hydrogen only, including an alkyl, alkenyl, alkynyl, cycloalkyl, alkylcycloalkyl, aryl, and aralkyl group in accordance with the above definitions.

The activity and more specifically the bioactivity of the compounds according to the present invention can be assessed using appropriate assays known to those skilled in the art, e.g. in vitro or in vivo assays. For instance, the protease inhibition activity against cysteine proteases may be determined according to the procedure of Garcia-Carreño, Biotechnology Education 1992, 3: 145-150, Hiwasa et al., Cancer letters 1993, 69: 161-165 or Giroud et al, ChemMedChem 2017, 12, 257-270, as utilised in the below, which are thus embodiments of standard in vitro assays.

Preferred is a compound of formula (I) and formula (IA), or a pharmacologically acceptable salt thereof, wherein the compound, or salt thereof, is a synthetic compound or a not naturally occurring derivative.

Further preferred is a compound of formula (I), or a pharmacologically acceptable salt thereof, wherein R¹ is a hydrogen atom, —OH or a C₁₋₃ alkoxy group, preferably a methoxy (—OCH₃) or ethoxy (—OC₂H₅) group; R¹ may also represent a C₃⁻ or C₄ cycloalkyl group, which may be optionally substituted; —NH₂, —NHCH₃ or —N(CH₃)₂.

In the compound of formula (I), or a pharmacologically acceptable salt thereof, R² can be a hydrogen atom, a group of formula —C(═NH)NH₂, a group of formula —C(═O)CH₃, or a group of formula —C(═O)CH₂CH₂CH₃.

In the compound of formula (I), or a pharmacologically acceptable salt thereof, n can preferably be 3 or 4.

In the compound of formula (I), or a pharmacologically acceptable salt thereof, R³ can represent an amino acid side chain of a proteinogenic amino acid, including, for example, a hydrogen atom (glycine), a phenyl group (phenylalanine) or a 4-hydroxyphenyl group (tyrosine); or a group of formula (II):

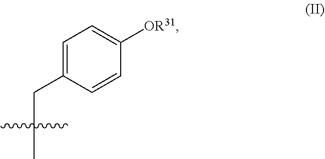

wherein R³¹ represents a (C₁-C₆)alkyl, (C₂-C₆)alkenyl, or (C₂-C₆) alkinyl group, all of which groups may optionally be substituted. Preferably, R³¹ represents an optionally substituted (C₁-C₄)alkyl, (C₂-C₄)alkenyl, or (C₂-C₄) alkinyl group; and more preferably R³¹ is selected from methyl, prop-2-enyl, prop-2-inyl, but-3-enyl and but-3-inyl.

In the compound of formula (I), or a pharmacologically acceptable salt thereof, R⁴ can be a C₁₋₇ alkyl or C₂-7 alkenyl group; which groups may optionally be substituted. Preferably, R⁴ is selected from methyl, heptyl, prop-1-enyl and hept-1-enyl.

As regards the compound of formula (IA), or a pharmacologically acceptable salt thereof, R^{1A} can preferably be a hydrogen atom, —OR¹¹, or —NR¹¹R¹²; wherein

R¹¹ and R¹² each, independently of one another, represents a hydrogen atom, or a (C₁-C₆)alkyl, (C₂-C₄)alkenyl, (C₂-C₄) alkinyl or (C₁-C₅)heteroalkyl group, or R¹¹ and R¹² together with the nitrogen atom to which they are attached form a 5- or 6-membered heterocyclic or heteroaromatic ring that can be substituted with from 0 to 3 substituents which substituents are each independently selected from halogen atom, —OH, —NH₂, —NHC₁₋₃ alkyl, and —N(C₁₋₃alkyl)₂.

More preferably, R^{1A} can represent —NH₂, —NHCH₃, —N(CH₃)₂, —NH(OC₁₋₃ alkyl), —NCH₃(OC₁₋₃ alkyl), —NH(C₁₋₃alkyl)CN;

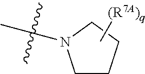

wherein each R^{7A} is independently selected from halogen atom, —OH, —NH₂, and —NHC₁₋₃ alkyl, and q is an integer of from 0 to 3;

a C₁₋₃ alkoxy group; or —OH. Particularly preferred is a compound, wherein R^{1A} is —NH₂, —NHCH₃, —N(CH₃)₂, —N(H)CH₂CN, —OCH₃, or —OC₂H₅.

In the compound of formula (IA), or a pharmacologically acceptable salt thereof, R^{2A} can be a hydrogen atom, a group of formula —C(═NH)NH₂, a group of formula —C(═O)CH₃, a group of formula —C(═O)CH₂CH₂CH₃.

In the compound of formula (IA), or a pharmacologically acceptable salt thereof, p can preferably be 3 or 4.

In the compound of formula (IA), or a pharmacologically acceptable salt thereof, R^{3A} can represent an amino acid side chain of a proteinogenic amino acid, including, for example, a hydrogen atom (glycine), a phenyl group (phenylalanine) or a 4-hydroxyphenyl group (tyrosine); or a group of formula (II):

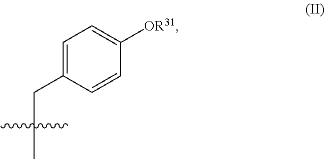

wherein R³¹ represents a (C₁-C₆)alkyl, (C₂-C₆)alkenyl, or (C₂-C₆) alkinyl group, all of which groups may optionally be substituted. The amino acid side chain of the proteinogenic amino acid can be optionally substituted. Preferred examples of the amino acid side chain of a proteinogenic amino acid include the side chains of tyrosine, phenylalanine, leucine and isoleucine.

Preferably, R³¹ represents an optionally substituted (C₁-C₄)alkyl, (C₂-C₄)alkenyl, or (C₂-C₄) alkinyl group; and more preferably R³¹ is selected from methyl, prop-2-enyl, prop-2-inyl, but-3-enyl and but inyl.

Preferably, the amino acid side chain of a proteinogenic amino acid is a side chain of tyrosine, phenylalanine, leucine and isoleucine. Also preferred is a derivative of tyrosine according to formula (II).

Further preferred, R^{3A} can represent the amino acid side chain of phenylalanine, leucine and isoleucine, wherein 1 to 3 H atoms in the respective side chain group may, independently of each other, be replaced by a halogen atom, OH, NH₂, —NHCH₃, —N(CH₃)₂, —NH(OC₁₋₃ alkyl), unsubstituted C₁-C₃alkyl, (C₁-C₃)haloalkyl, (C₁-C₃)hydroxyalkyl, or (C₁-C₃)alkoxy group.

Particularly preferred is a compound, wherein R^{3A} is:

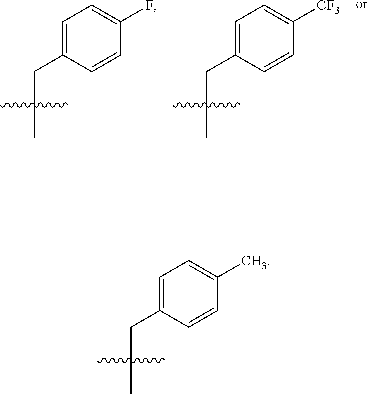 [SMILES: *Cc1ccc(F)cc1 ; *Cc1ccc(C(F)(F)F)cc1 ; "or" ; *Cc1ccc(C)cc1]

In the compound of formula (IA), or a pharmacologically acceptable salt thereof, R^{4A} can be a hydrocarbon group containing 1 to 12 carbon atoms or a heteroaryl group containing from 5 to 10 ring atoms, and, optionally, 1 to 3 H atoms in the hydrocarbon and the heteroaryl group may, independently of each other, be replaced by a halogen atom, OH, NH₂, —NHCH₃, —N(CH₃)₂, —NH(OC₁₋₃ alkyl), unsubstituted C₁-C₃alkyl, (C₁-C₃)haloalkyl, (C₁-C₃)hydroxyalkyl, or (C₁-C₃)alkoxy group.

Preferably, R^{4A} can be a C₁₋₇ alkyl, C₂-7 alkenyl, (C₂-C₇) alkynyl, cyclohexyl, phenyl, benzyl or pyridyl group; which groups may optionally be substituted, e.g. 1 to 3 H atoms in said groups may, independently of each other, be replaced by a halogen atom, OH, NH₂, —NHCH₃, —N(CH₃)₂, —NH(OC₁₋₃ alkyl), unsubstituted C₁-C₃alkyl, (C₁-C₃)haloalkyl, (C₁-C₃)hydroxyalkyl, or (C₁-C₃)alkoxy group. More preferably, R^{4A} is selected from methyl, heptyl, octyl, prop-1-enyl and hept-1-enyl, oct-1-enyl.

Particularly preferred, R^{4A} can be a methyl, heptyl, prop-1-enyl, hept-1-enyl, cyclohexyl, phenyl, or pyridyl group.

Also preferred is a compound, or a pharmacologically acceptable salt thereof, wherein R^{4A} is a C₁₋₇ alkyl or C₂-7 alkenyl group; which groups may optionally be substituted. Preferably, R^{4A} is selected from methyl, heptyl, prop-1-enyl and hept-1-enyl.

In the compound of formula (IA), or a pharmacologically acceptable salt thereof, R^{5A} and R^{6A} can be a hydrogen atom. In the compound of formula (IA), or a pharmacologically acceptable salt thereof, R^{5A} can be a methyl group and R^{6A} can be a hydrogen atom. In the compound of formula (IA), or a pharmacologically acceptable salt thereof, R^{5A} can be a hydrogen atom and R^{6A} can be a methyl group.methyl group. In the compound of formula (IA), or a pharmacologically acceptable salt thereof, R^{5A} and R^{6A} can be a methyl group.

The compound of formula (I), or a pharmacologically acceptable salt thereof, includes the compounds depicted below:

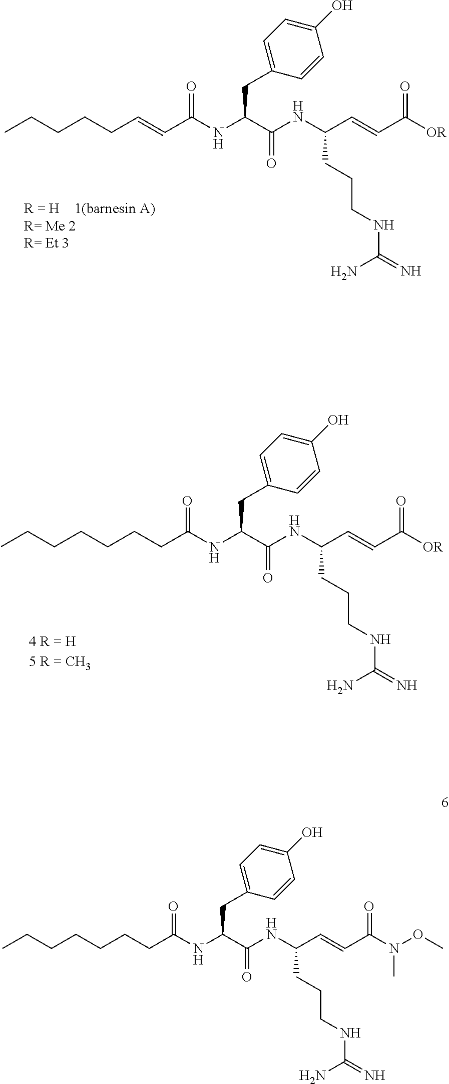

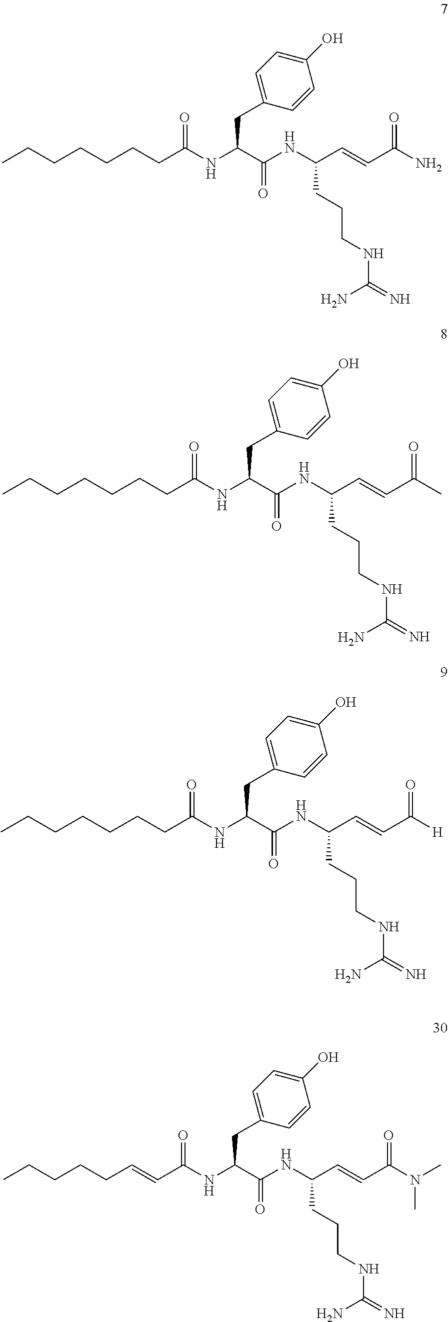

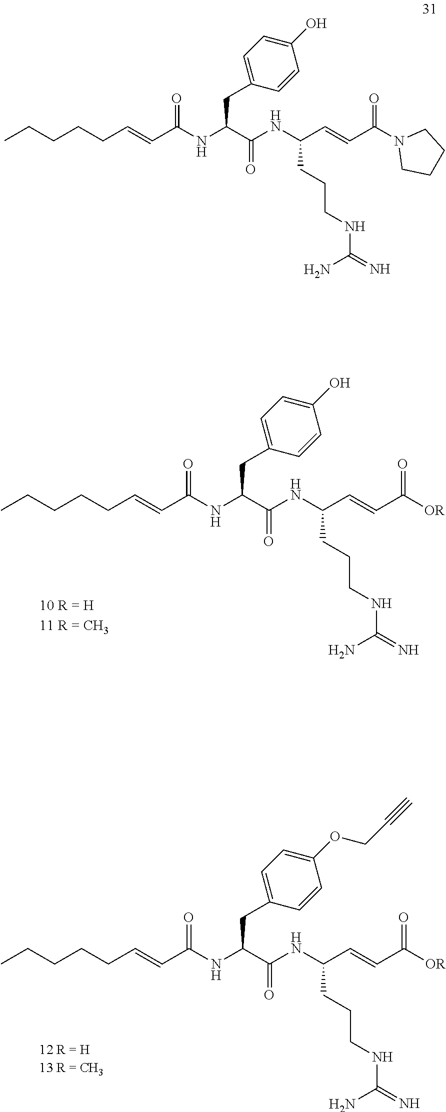

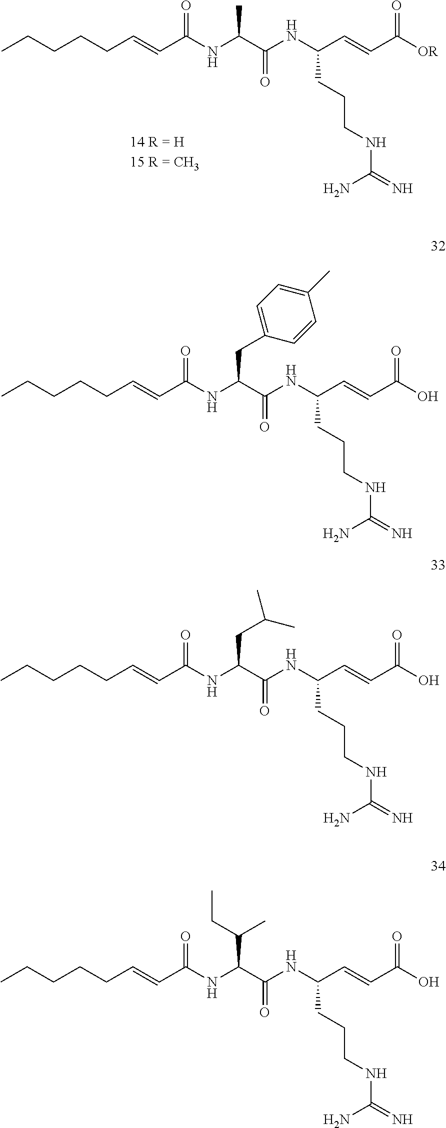

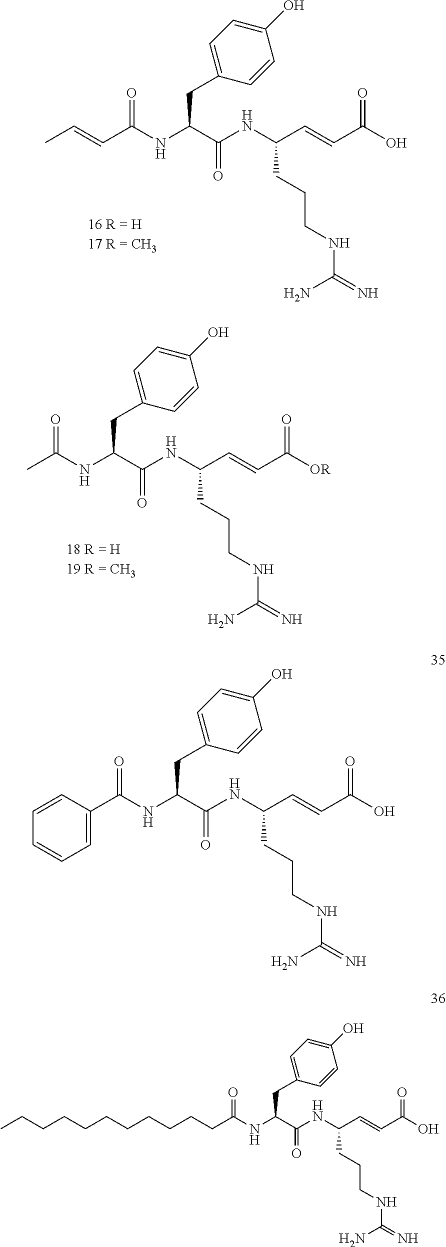

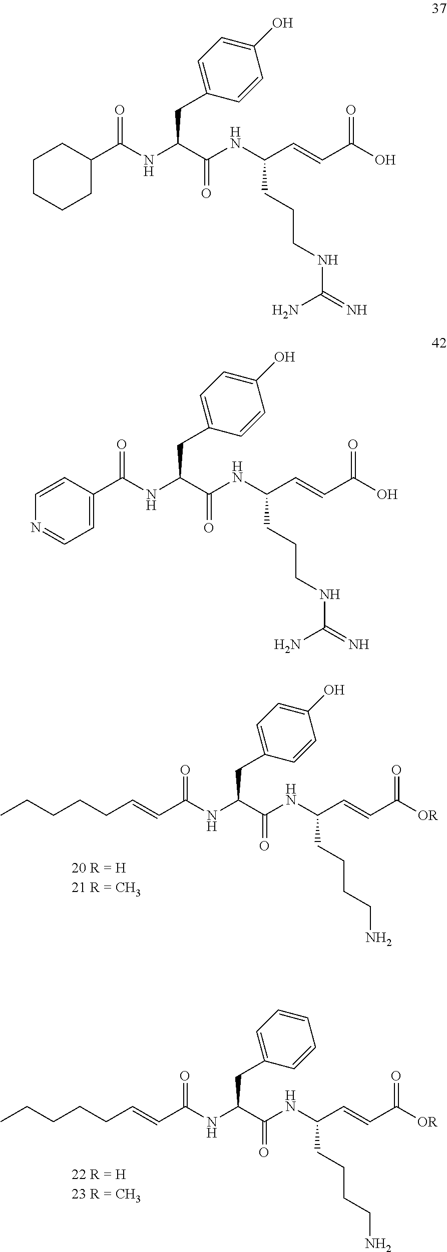

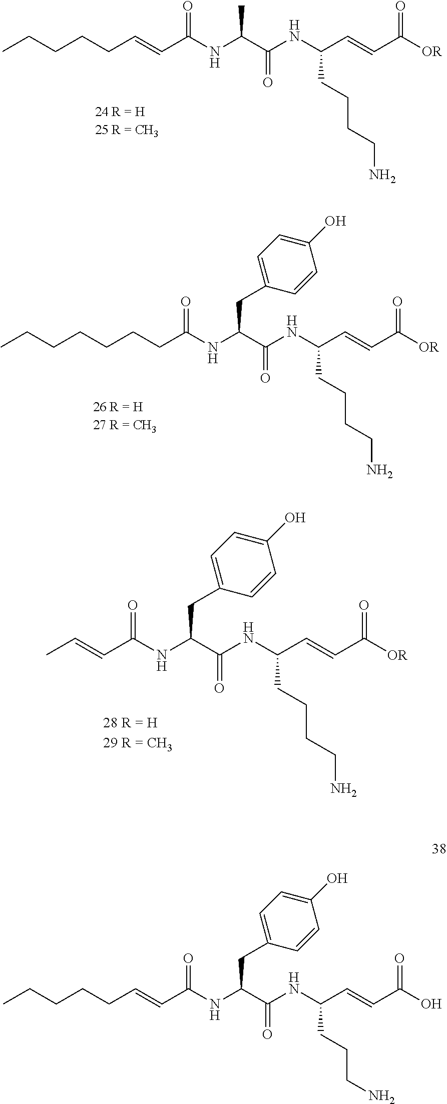

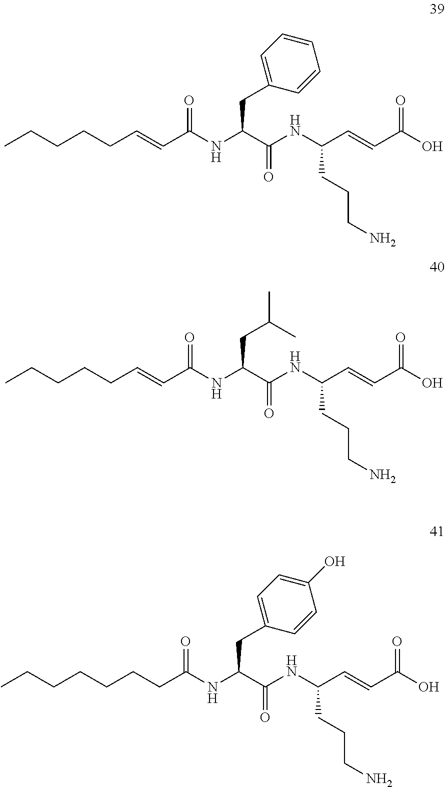 [SMILES: CCCCC/C=C/C(=O)N[C@@H](Cc1ccccc1)C(=O)N[C@H](/C=C/C(=O)O)CCCN ; CCCCC/C=C/C(=O)N[C@@H](CC(C)C)C(=O)N[C@H](/C=C/C(=O)O)CCCN ; CCCCCCCC(=O)N[C@@H](Cc1ccc(O)cc1)C(=O)N[C@H](/C=C/C(=O)O)CCCN]

The therapeutic use of a compound of formula (I), of a pharmacologically acceptable salt, solvate or hydrate thereof, and also of a formulation and/or pharmaceutical composition containing the same is within the scope of the present invention. The present invention also relates to the use of a compound of formula (I) as active ingredient in the preparation or manufacture of a medicament.

A pharmaceutical composition according to the present invention comprises at least one compound of formula (I) and, optionally, one or more carrier substance(s), excipient(s) and/or adjuvant(s). Pharmaceutical compositions may additionally comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Furthermore, one or more other active ingredients may (but need not) be included in the pharmaceutical compositions provided herein. For instance, the compounds of the invention may advantageously be employed in combination with another different antibiotic, an antifungal agent, an anti-viral agent, an anti-histamine, a non-steroidal anti-inflammatory drug, a disease modifying anti-rheumatic drug, a cytostatic drug, a drug with smooth muscle activity modulatory activity; or mixtures of the aforementioned.

A pharmaceutical composition may be formulated for any appropriate route of administration, including, for example, parenteral administration. The term parenteral as used herein includes subcutaneous, intradermal, intravascular such as, e.g., intravenous, intramuscular, spinal, intracranial, intrathecal, intraocular, periocular, intraorbital, intrasynovial and intraperitoneal injection, as well as any similar injection or infusion technique.

Carrier substances are, for example, cyclodextrins such as hydroxypropyl O-cyclodextrin, micelles or liposomes, excipients and/or adjuvants. Customary excipients include, for example, inert diluents such as, e.g., calcium carbonate, sodium carbonate, lactose, calcium phosphate or sodium phosphate, granulating and disintegrating agents such as, e.g., corn starch or alginic acid, binding agents such as, e.g., starch, gelatin or acacia, and lubricating agents such as, e.g., magnesium stearate or stearic acid. Examples of adjuvants are aluminum hydroxide, aluminum phosphate, calcium phosphate hydroxide, paraffin oil, squalene, thimerosal, detergents, Freund's complete adjuvant, or Freund's incomplete adjuvant.

For the prevention and/or treatment of conditions or disorders associated with a pathophysiological level of a cysteine protease, including neurodegenerative disorders, e.g. Alzheimer's disease; parasitic infections, e.g. Chagas disease and human African trypanosomiasis; and invasive and metastatic cancers, the dose of the biologically active compound according to the invention may vary within wide limits and may be adjusted to individual requirements. Active compounds according to the present invention are generally administered in a therapeutically effective amount. The expression “therapeutically effective amount” denotes a quantity of the compound(s) that produces a result that in and of itself helps to ameliorate, heal, or cure the respective condition or disease. Preferred doses range from about 0.1 mg to about 140 mg per kilogram of body weight per day (about 0.5 mg to about 7 g per patient per day). The daily dose may be administered as a single dose or in a plurality of doses. The amount of active ingredient that may be combined with the carrier materials to produce a single dosage form will vary depending upon the host treated and the particular mode of administration. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

The invention further relates to a combination preparation containing at least one compound according to the invention and at least one further active pharmaceutical ingredient. The combination preparation of the invention can be used as a medicament, in particular in the treatment or prophylaxis of conditions or disorders associated with a pathophysiological level of a cysteine protease, including neurodegenerative disorders, e.g. Alzheimer's disease; parasitic infections, e.g. Chagas disease and human African trypanosomiasis; and invasive and metastatic cancers. Preferably, in the combination preparation of the invention the further active pharmaceutical ingredient can be selected from an antibiotic, an anticancer drug, dopaminergic substances, cholinesterase inhibitors, antipsychotic drugs, analgesic drugs for pain, anti-inflammatories for infections, non-steroidal anti-inflammatory drugs for Alzheimer's disease, oxaboroles, fexinidazole, suramin, pentamidine, benznidazole, and nifurtimox.

Preferred compounds of the invention will have certain pharmacological properties. Such properties include, but are not limited to, bioavailability (especially with regard to oral administration), metabolic stability and sufficient solubility, such that the dosage forms can provide therapeutically effective levels of the compound in vivo.

The compound according to the invention, or a pharmacologically acceptable salt thereof, as well as the pharmaceutical composition or combination preparation according to the invention, can be used as a medicament, which can be administered to a patient, e.g. parenterally to a human or an other mammal, with dosages as described herein, and will be present within at least one body fluid or tissue of the patient. As used herein, the term “treatment” encompasses both disease-modifying treatment and symptomatic treatment, either of which may be prophylactic, i.e., before the onset of symptoms, in order to prevent, delay or reduce the severity of symptoms, or therapeutic, i.e., after the onset of symptoms, in order to reduce the severity and/or duration of symptoms. In particular, the conditions or diseases that can be ameliorated, prevented or treated using a compound of formula (I), a pharmaceutical composition or a combination preparation according to the invention include conditions or disorders associated with a pathophysiological level of a cysteine protease, including neurodegenerative disorders, e.g. Alzheimer's disease; parasitic infections, e.g. Chagas disease and human African trypanosomiasis; and invasive and metastatic cancers. Thus, the present invention also provides methods for treating patients suffering from said diseases. A method for the treatment of a subject which is in need of such treatment comprises the administration of a compound, a pharmaceutical composition, or a combination preparation according to the invention. The term “subject” refers to patients, including, but not limited to primates (especially humans), domesticated companion animals (such as dogs, cats, horses) and livestock (such as cattle, pigs, sheep).

The compound according to the invention, or a pharmacologically acceptable salt thereof, can also have utility as an inhibitor of a proteasome or a cysteine protease, e.g. in in vivo or in vitro assays. Preferably, the cysteine protease is a cathepsin, including cathepsin B, C, F, H, K, L, O, S, V, X and W, and isoforms thereof; a calpain, including calpain 1 to calpain 15, and isoforms thereof; papain, ficin, a falcipain, including falcipain 1 to falcipain 4, and isoforms thereof; rhodesain (cathepsin L-like protease), and cruzain.

The present invention also provides a synthetic (not naturally occurring) nucleic acid sequence encoding a nonribosomal peptide-synthetase (NRPS)-polyketide synthase (PKS) biosynthetic hybrid cluster capable of synthesizing barnesin A (1), wherein the sequence has a sequence identity to the full-length sequence of SEQ ID NO. 1 from at least 90%, 95%, 96%, 97%, 98%, 98.5%, 99%, or 99.5% to 100%.

The synthetic nucleic acid sequence is a variant derived from the native NRPS-PKS hybrid cluster of Sulfurospirillum barnesii which includes cDNAs and may further comprise regulatory sequences, such as promoter and translation initiation and termination sequences, and can further include sequences that facilitate stable maintenance in a host cell, i.e., sequences that provide the function of an origin of replication or facilitate integration into host cell chromosomal or other DNA by homologous recombination. The term “variant” as used herein denotes a polynucleotide that has been modified at one or more positions compared to the native NRPS-PKS hybrid cluster of Sulfurospirillum barnesii. Nucleic acids can be, relative to the native NRPS-PKS hybrid cluster, substituted (different), inserted, or deleted, but the variant has generally similar (enzymatic) activity or function as compared to the native NRPS-PKS hybrid cluster.

The term “identity” refers to a property of sequences that measures their similarity or relationship. Identity is measured by dividing the number of identical residues by the total number of residues and multiplying the product by 100.

The term “synthetic” as used herein means that the material, e.g. a nucleic acid sequence, has been synthesized in vitro by well-known chemical synthesis/recombination techniques, and further that the nucleic acid sequence is flanked to a “backbone” nucleic acid to which it is not adjacent in its natural environment. Backbone molecules according to the invention include nucleic acids such as cloning and expression vectors, self-replicating nucleic acids, viruses, integrating nucleic acids and other vectors or nucleic acids used to maintain or manipulate a nucleic acid insert of interest. Recombinant polypeptides of the invention, generated from these nucleic acids can be individually isolated or cloned and tested for a desired activity. Any recombinant expression system can be used, including bacterial, mammalian, yeast, insect or plant cell expression systems.

Also the following methods for producing a compound of formula (I) lie within the scope of the present invention: chemical synthesis, semisynthesis, and biosynthesis including recombinant techniques.

For instance, a compound of general formula (I) can be produced by a method comprising the steps:
(a) fermenting Sulfurospirillum barnesii (DSM 10660); and
(b) separating and retaining the compound according to general formula (I) from the culture broth.

It is understood that the production of compounds of formula (I) is not limited to the use of the particular organism described herein, which is given for illustrative purpose only. The invention also includes the use of any mutants which are capable of producing a compound of formula (I) including natural mutants as well as artificial mutants, e.g. genetically manipulated mutants and the expression of the gene cluster responsible for biosynthesis in a producer strain or by heterologous expression in host strains.

The culturing step can be performed in liquid culture, by growing the respective host cell in media containing one or several different carbon sources, and one or different nitrogen sources. Also salts are essential for growth and production. Suitable carbon sources are different mono-, di-, and polysaccharides like maltose, glucose or carbon from amino acids like peptones. Nitrogen sources are ammonium, nitrate, urea, chitin or nitrogen from amino acids. The following inorganic ions support the growth or are essential in synthetic media: Mg-ions, Ca-ions, Fe-ions, Mn-ions, Zn-ions, K-ions, sulfate-ions, Cl-ions, phosphate-ions. The host cell may be a microorganism, e.g. Sulfurospirillum barnesii strain SES-3 (DSM 10660, Genbank accession CP003333.1). Temperatures for growth and production are between 15° C. to 40° C., preferred temperatures are between 25° C. and 35° C., especially at 28° C. The pH of the culture solution is from 5 to 8, preferably a pH of 7.2 to 7.4.

A compound of general formula (I) can also be obtained by chemical synthesis in a number of ways well known to one skilled in the art of organic synthesis using usual chemical reaction and synthesis methods. For example, the compounds of the present invention can be synthesized according to Reaction Schemes 1 and 2 shown below using synthetic methods known in the art of synthetic organic chemistry, or variations thereon as appreciated by those skilled in the art, e.g. March's Advanced Organic Chemistry: Reactions, Mechanisms, and Structure, Michael B. Smith, Jerry March, John Wiley & Sons, 2007. Unless indicated otherwise, all variables have the above defined meaning. As starting materials reagents of standard commercial grade can be used without further purification, or can be readily prepared from such materials by routine methods. Those skilled in the art of organic synthesis will recognize that starting materials and reaction conditions may be varied including additional steps employed to produce compounds encompassed by the present invention.

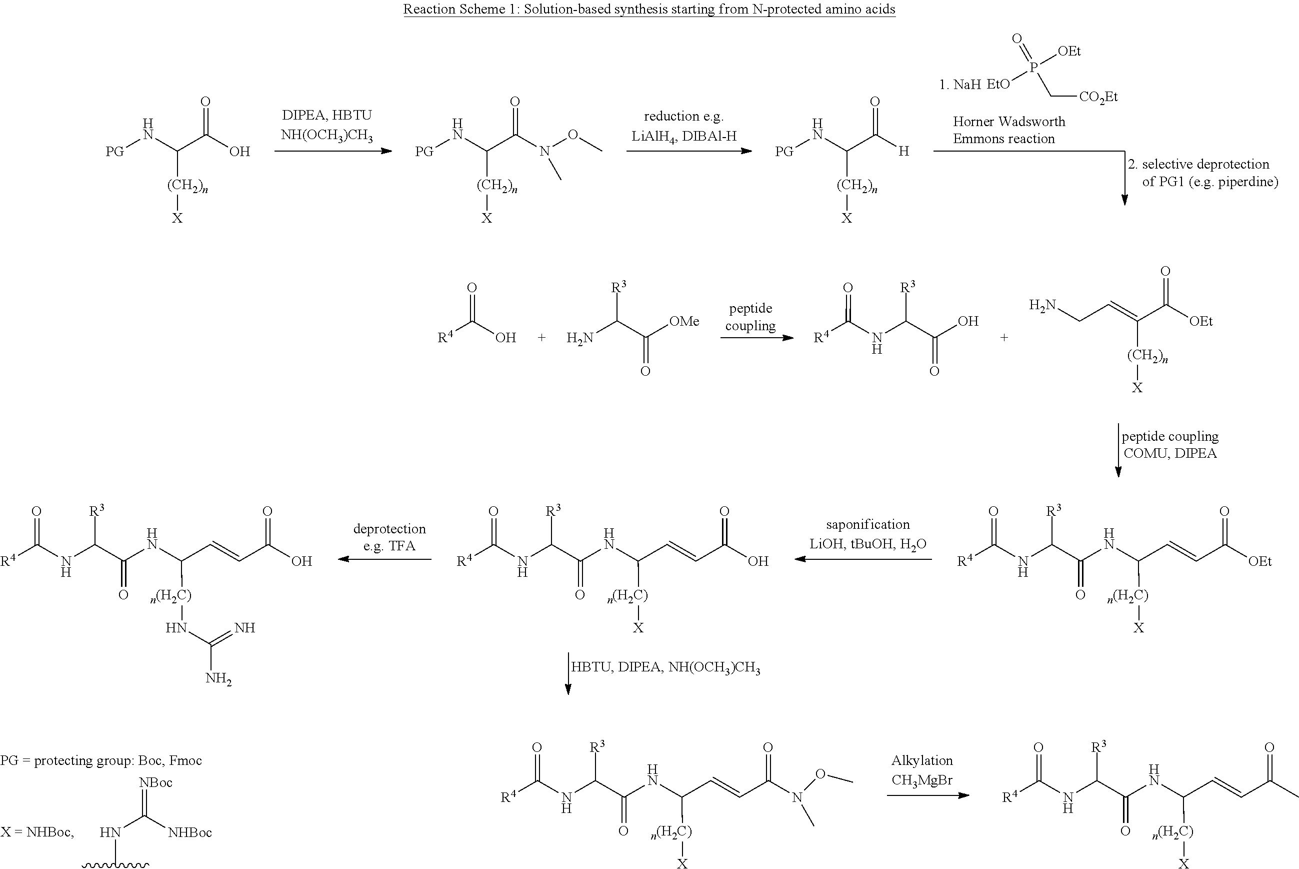

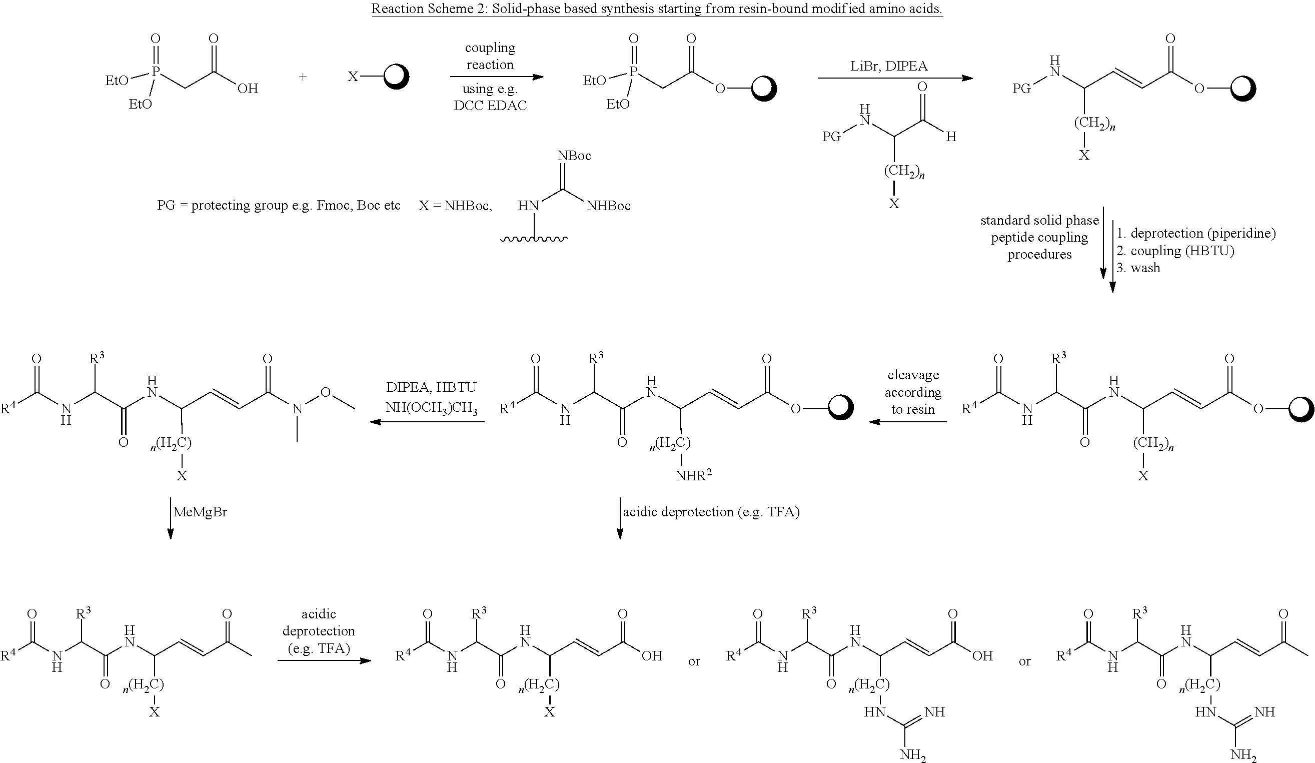

The present invention is now further illustrated by the following examples from which further features, embodiments and advantages of the present invention may be taken.

### EXAMPLES

### General Experimental Procedures

NMR measurements were performed on a Bruker AVANCE II 300 MHz, Bruker AVANCE III 500 MHz and a Bruker AVANCE III 600 MHz spectrometer, equipped with a Bruker Cryoplatform. The chemical shifts are reported in parts per million (ppm) relative to the solvent residual peak of DMSO-d₆ (¹H: 2.50 ppm, quintet; ¹³C: 39.52 ppm, heptet), CDCl₃ (¹H: 7.26 ppm, singlet; ¹³C: 77.16 ppm, triplet) and MeOD-d₄ (¹H: 3.31 ppm, quintet; ¹³C: 49.00 ppm, heptet). LC-ESI-HRMS measurements were carried out on an Accela UPLC system (Thermo Scientific) coupled with a Accucore C18 column (100×2.1 mm, particle size 2.6 μm) combined with a Q-Exactive mass spectrometer (Thermo Scientific) equipped with an elecrospray ion (ESI) source. UHPLC-MS measurements were performed on a Shimadzu LCMS-2020 system equipped with single quadrupole mass spectrometer using a Phenomenex Kinetex C18 column (50×2.1 mm, particle size 1.7 μm, pore diameter 100 Å). Column oven was set to 40° C.; scan range of MS was set to m/z 150 to 2,000 with a scan speed of 10,000 u/s and event time of 0.25 s under positive and negative mode. DL temperature was set to 250° C. with an interface temperature of 350° C. and a heat block of 400° C. The nebulizing gas flow was set to 1.5 L/min and dry gas flow to 15 L/min. Semi-preparative HPLC was performed on a Shimadzu HPLC system using a Phenomenex Luna C18(2) and phenyl-hexyl 250×10 mm column (particle size 5 μm, pore diameter 100 Å), or a Biotage Isolera Prime. IR spectra were recorded on an FT/IR-4100 ATR spectrometer (JASCO). Optical rotations were recorded in MeOH on a P-1020 polarimeter (JASCO). Solid phase extraction was carried out using Chromabond C18ec cartridges filled with 1 g and 10 g of octadecyl-modified silica gel (Macherey-Nagel, Germany). PCR was performed on a Peqstar 2× Gradient cycler.

Chemicals: Methanol (VWR, Germany); water for analytical and preparative HPLC (Millipore, Germany), formic acid (Carl Roth, Germany); acetonitrile (VWR as LC-MS grade), media ingredients (Carl Roth, Germany). All reagents and solvents for synthesis were purchased from Acros Organics, Alfa Aesar, Carbolution Chemicals, Carl Roth, Sigma Aldrich, TCI, Th. Geyer and VWR and used without further purification.

### Example 1 Biosynthesis and Biophysical Analysis of a Compound According to General Formula (I) of the Present Invention—Barnesin A (1)

Cultivation of Sulfurospirillum barnesii strain SES-3 (DSM 10660): Sulfurospirillum spp. were generally cultivated at 28° C. using the following conditions: For solid phase cultivation, S. barnesii was grown microoxically on R2A agar plates incubated in an anaerobic jar with approximately 0.2% oxygen in the gas phase for at least one week. When grown anaerobically, a defined mineral growth medium as described for S. multivorans was used with the following modifications: vitamin B12 (cyanocobalamin) and resazurine were omitted and pyruvate (40 mM) was used as electron donor and fumarate (40 mM) as electron acceptor. Small scale cultivations (100 mL) were performed in 200 mL rubber-stoppered serum bottles, large-scale cultivations in 2 L rubber-stoppered Schott bottles containing 1 L medium. Glass bottles used for cultivation were capped with Teflon-coated butyl rubber septa. Growth was monitored photometrically by measuring the optical density at 578 nm. Microaerobic cultivation with the aforementioned medium was performed using 2 L Schott bottles with an initial addition of 2% sterile air into the gas phase into the medium without fumarate as electron acceptor. Inoculation of the medium was performed with 10% of a preculture cultivated until the exponential phase.

Isolation and characterization: Cultures were centrifuged at 4000 rpm for 20 min and RT. The cell pellet was harvested and extracted using MeOH (4° C., overnight), cell debris was filtered off and methanolic extracts added to metabolites at a later step. The culture supernatant was mixed with activated HP20/XAD4 (1:1) resin and stirring overnight (4° C., 300 rpm). Then, the resin was filtered off, washed with 20% MeOH (if not mentioned otherwise, mixtures refers to MeOH in ddH₂O) and metabolites eluted with 50% MeOH and 100% MeOH. Then methanolic cell pellet extracts were added to the 100% MeOH resin eluate. Combined extracts were concentrated under reduced pressure and redissolved in 10% MeOH. The crude extract was loaded on an activated and equilibrated SPE-C18 cartridge and fractionated using 50% and 100% MeOH. The concentrated 100% MeOH fraction was purified first by a semi-preparative HPLC over Luna C18(2) column to obtain subfractions (Fr. 1-34) using the following gradient: 0-3 min, 50% B, 3-30 min 50-100% B, 30-35 min 100% B, 35-41 min 100-50% B with a flow rate of 2.0 mL/min (A: ddH₂O with 0.1% formic acid; B: MeOH). Concentrated fractions 10-12 were further separated by a second semi-preparative HPLC run over Phenyl-Hexyl column to yield pure barnesin A (1, 1.0 mg, t_{R}=25.6 min) with the following gradient: 0-5 min 10% D, 5-38 min 10-75% D, 38-40 min 75-100% D, 40-45 min 100% D, 45-50 min 100-10% D (C: ACN; D: NH₄OAc 20 mM, pH 7.0) with a flow rate of 2.0 mL/min. The samples were analyzed using analytical HPLC over Phenyl-Hexyl column (250×4.6 mm) or Luna C18(2) with the following gradient: 0-3 min 50% B, 3-30 min 50-100% B, 30-35 min 100% B, 37-40 min 100-50% B, 35-41 min 50% B (A: ddH₂O with 0.1% formic acid; B: MeOH) (1, t_{R}=14.38 min).

Marfey's reaction: Barnesin A (1, 0.2 mg) was hydrolyzed by 6 N HCl (1.0 mL) at 110° C. overnight (15 h). HCl was removed using SpeedVac (42° C.) and FDAA (1-fluoro-2,4-dinitrophenyl-5-L-alanine amide, 20 μL, 10 mg/mL in acetone) and NaHCO₃ (100 μL, 1 M) were added. The reaction was performed under 80° C. for 10 min. The reaction was quenched by adding of HCl (50 μL, 2 N) and D-tyrosine reference were converted accordingly under the same condition. After centrifugation for 15 min at 13,000 rpm the supernatant was analyzed by LC-MS. 1 μL of the reaction was injected and analyzed using the following gradient: 0-1 min 10% D, 1-7 min 70% D, 7-10 100% D, 10-13.5 min 10% D (B: ddH₂O with 0.1% formic acid; D: MeCN with 0.1% formic acid) at a flow rate of 0.7 mL/min.

Barnesin A (1): colourless solid; [α]_{D}²⁵−5.76 (c 0.0910, MeOH); UV (MeOH): λₘₐₓ 196; 214 nm; IR (ATR) vₘₐₓ: 1240, 1385, 1455, 1545, 1655, 2365, 2860, 2930, 3070, 3260 cm⁻¹; ESI-HRMS: m/z: 488.2864 [M+H]⁺; calcd. 488.2867.

Structure assignment: The molecular formula of barnesin A (1) was assigned as C₂₅H₃₇O₅N₅ based on ESI-HRMS analysis (m/z 488.2864 [M+H]⁺, calcd. 488.2867, Δ=−0.69 ppm) and confirmed by the observation of 25 carbon signals from ¹³C-NMR spectrum, including 11 olefinic or aromatic carbons, between δ_{C} 114.8 ppm and δ_{C} 157.4 ppm, three carbonyl carbons at δ_{C} 164.7 ppm, 171.1 ppm and 171.4 ppm, three nitrogen-bearing carbons (δ_{C} 39.9 ppm, 48.6 ppm, and 54.6 ppm), and eight aliphatic carbons between δ_{C} 13.9 ppm and 37.4 ppm. Detailed analysis of the obtained 2D NMR spectra (DMSO-d₆) revealed the structure of a modified di-peptides consisting of a tyrosine moiety, a modified arginine moiety and a fatty acid tail. The di-peptide was identified based on the presence of two α-carbons (δ_{H} 4.47 ppm/δ_{C} 54.6 ppm and δ_{H} 4.34 ppm/δ_{C} 48.6 ppm). The tyrosine moiety was assigned based on COSY correlations of NH(3) δ_{H} 8.02 ppm) to H-10 δ_{H} 4.47 ppm) to H-11 δ_{H} 2.87/2.56 ppm, δ_{C} 37.4 ppm), and correlations of the para-substituted aromatic protons H-13 δ_{H} 6.99 ppm/δ_{C} 130.0 ppm) to H-14 δ_{H} 6.60 ppm/δ_{C} 114.8 ppm). This assignment was confirmed by the HMBC correlations of H-10 to C-9/C-11/C-12, H-11 to C-9/C-10/C-12/C-13, H-13 to C-11/C-14/C-15 and H-14 to C-12/C-15. Furthermore, two unsaturated (trans, J=15.4 Hz and 15.7 Hz. respectively) spin systems were observed, one of which correlated to an α,β-unsaturated carbonyl group of a modified γ-amino acid connected to the tyrosine moiety (COSY correlation of NH(2) to H-4 and HMBC correlation of NH(2) to C-9). This was supported by COSY correlations from H-2 to H-3 to H-4 to H-5 to H-6 to H-7 and NH(1), and corresponding HMBC correlations of H-2/H-3 to C-1 (δ_{C} 171.4 ppm). The presence of guanidine moiety was deduced based on the chemical shift of quaternary carbon at C-8 (δ_{C} 157.4 ppm) together with the requirement of molecular formula and unsaturation degree, as well as HMBC correlation of H-7 to C-8. The second unsaturated spin system belonged to an unsaturated fatty acid moiety (COSY correlations from olefinic proton H-17 OH 5.89 ppm/δ_{C} 124.3 ppm) to H-18 δ_{H} 6.49 ppm/δ_{C} 142.6 ppm) and to methylene protons H-19, H-20, H-21, H-22, and the presence of a methyl group δ_{H} 0.86 ppm/δ_{C} 13.9 ppm). HMBC correlations of H-17/H-18/NH(3) to C-16 revealed the connection to the N-terminus of assigned tyrosine moiety. The structure assignment was further confirmed by ESI-HR-MS/MS fragmentation, which revealed the fragment ion pair of m/z at 125.0964 and 364.1966 corresponding to the amine bond cleavage on the N-terminal of tyrosine moiety. The ion pair of m/z at 288.1583 and 201.1339 indicated the amine bond cleavage on the C-terminal of tyrosine moiety.

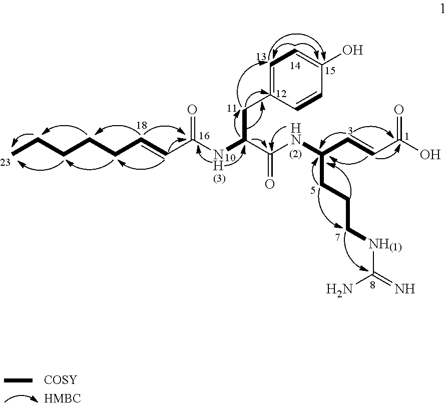

In summary, on the basis of the obtained NMR data, the compound was named according to its producing organism: barnesin A (1).

TABLE 1
NMR data of barnesin A (1) (DMSO-d₆, at 300K)^{a}.

| Position | δ_{C}, type^{b} | δ_{H}, mult. (J in Hz) | COSY | HMBC | NOESY |
|---|---|---|---|---|---|
| 1 | 171.4, qC | | | | |
| 2 | 127.8, CH | 5.73, d (15.7) | 3 | 1, 4 | 3, 4, 10, 11b, NH(2) |
| 3 | 141.2, CH | 6.42, dd | 2, 4 | 1, 2, 4 | 2, 4, 5b, 6, NH(2) |
| | | (15.7, 5.0) | | | |
| 4 | 48.6, CH | 4.34, br s | 3, 5a, 5b, NH(2) | | 2, 3, 5b, 6, 7a, 7b, NH(2) |
| 5a | 30.7, CH2 | 1.36, m | 4, 5b | 4, 7 | 3, 4, 5b, 7a, NH(2) |
| 5b | | 1.68, m | 4, 5a, 6 | | 3, 4, 6, 7a, NH(2) |
| 6 | 24.9, CH₂ | 1.46, m | 5b, 7a, 7b | 4, 5, 7 | 3, 4, 5b, 7a, NH(2) |
| 7a | 39.9, CH₂ | 3.05, m | 6 | 8 | 4, 6, 5b |
| 7b | | 3.09, m | 6 | | 4, 6, 5b |
| 8 | 157.4, qC | | | | |
| 9 | 171.1, qC | | | | |
| 10 | 54.6, CH | 4.47, t (8.4) | 11a, 11b, NH(3) | 9, 11, 12 | 6, 10, 11a, 11b, 13, NH(2), NH(3) |
| 11a | 37.4, CH₂ | 2.56, t (12.0) | 10, 11b | 9, 10, 12, 13 | 10, 11b, 13, NH(2), NH(3) |
| 11b | | 2.87, d (12.0) | 10, 11a | 9, 10, 12, 13 | 2, 10, 11a, 13, NH(2), NH(3) |
| 12 | 128.1, qC | | | | |
| 13 | 130.0, CH | 6.99, d (8.0) | 14 | 11, 14, 15 | 2, 10, 11a, 11b, 14, NH(1), NH(3) |
| 14 | 114.8, CH | 6.60, d (8.0) | 13 | 12, 15 | 13 |
| 15 | 155.7, qC | | | | |
| 16 | 164.7, qC | | | | |
| 17 | 124.3, CH | 5.89, d (15.4) | 18 | 16, 19 | 18, 19, NH(3) |
| 18 | 142.6, CH | 6.49, dd | 17, 19 | 16, 17, 19, 20 | 17, 19 |
| | | (15.4, 6.7) | | | |
| 19 | 31.1, CH₂ | 2.07, m | 18, 20 | 17, 18, 20, 21 | 17, 18 |
| 20 | 27.5, CH₂ | 1.36, m | 19, 21 | 18, 19, 22 | |
| 21 | 30.8, CH₂ | 1.24, m | 20, 22 | 19, 20, 22, 23 | |
| 22 | 21.9, CH₂ | 1.27, m | 21, 23 | 20, 21, 23 | |
| 23 | 13.9, CH₃ | 0.86, t (8.6) | 22 | 21, 22 | |
| NH(1) | | 10.09, br s | 7a, 7b | | |
| NH2) | | 8.50, d (8.1) | 4 | 9 | 2, 3, 4, 5b, 6, 7a, 10, 11a, 11b, 13, NH(3) |
| NH(3) | | 8.02, d (8.4) | 10 | 10, 11, 16 | 10, 11a, 11b, 13, 17, NH(3) |

^{a}600 MHz for ¹H NMR, COSY, HSQC and HMBC; 150 MHz for ¹³C NMR; 500 MHz for NOESY;
^{b}numbers of attached protons were determined by analysis of 2D spectra

### Example 2 Synthesis According to Reaction Scheme 1

Specific examples for the preparation of compounds of formula (I) are provided below. Unless otherwise specified all starting materials and reagents are of standard commercial grade, and are used without further purification, or are readily prepared from such materials by routine methods. Those skilled in the art of organic synthesis will recognize that starting materials and reaction conditions may be varied including additional steps employed to produce compounds encompassed by the present invention.

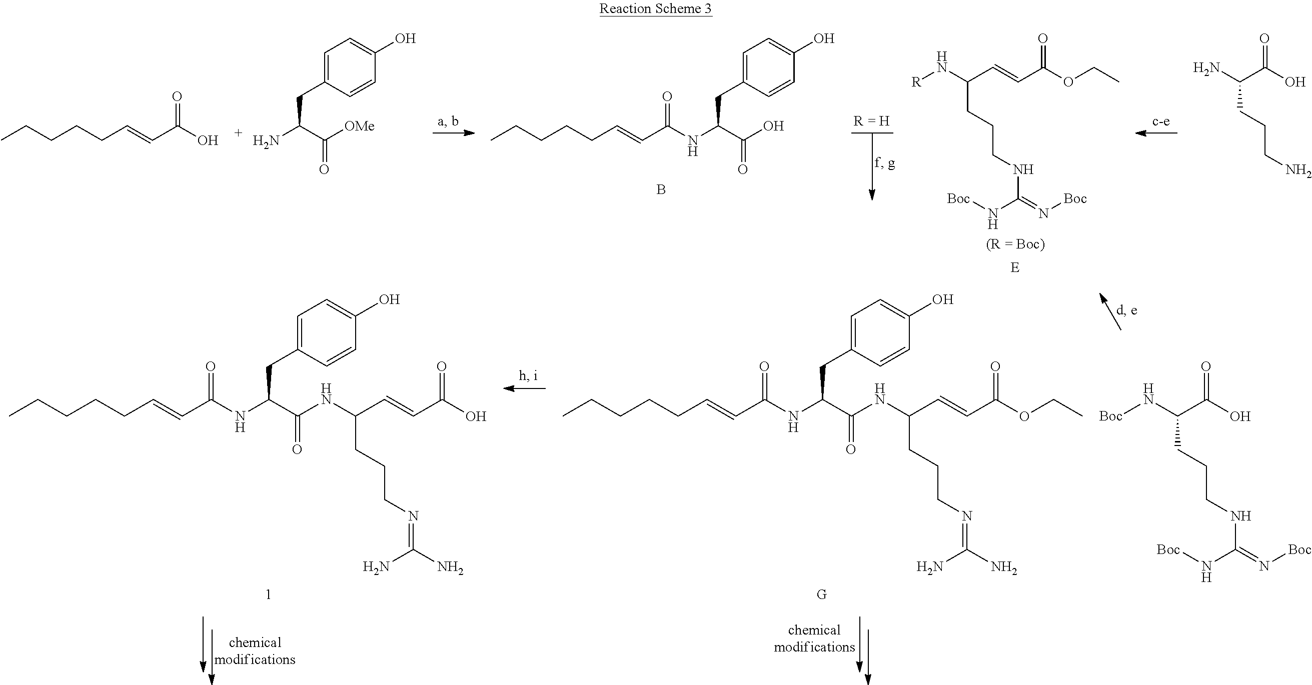

a) HBTU, DIPEA, DCM, r.t., o.n., 78%; b) 10%_{-aq} KOH, MeOH, r.t., 3 h, quant.; c) 1. CuCO₃·Cu(OH)₂, H₂O, r.t., 15 min, quant; 2. bis-Boc-pyrazolocarboxamidine, DIPEA, formamide, dioxane, r.t., o.n., 3. EDTA·2Na·2H₂O, NaHCO₃, H₂O then Fmoc-OSu, acetone, r.t., o.n. 70% (three steps); d) N,O-dimethylhydroxylamin hydrochlorid, HBTU, DIPEA, DCM, r.t., o.n. 71%; e) 1. LiAlH₄, THF, 0° C., 30 min, 2. triethyl phosphonoacetate, NaH, THF, 0° C., 45 min, 32% over two steps; f) 20% piperidine in DMF, r.t., 4 h, 86%; g) COMU, DIPEA, DMF, 0° C., 6 h, 27%; h) TFA, DCM, 0° C. to r.t., o.n.; i) porcine liver esterase, DMSO, H₂O, Tris/HCl buffer, 37° C., 5 d, 47% over two steps.

### 2.1. Methyl (E)-oct-2-enoyl-tyrosinate (A)

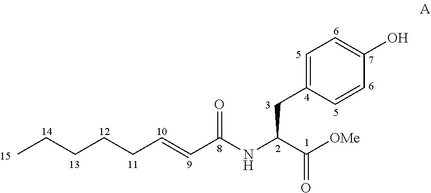 [SMILES: CCCCC/C=C/C(=O)N[C@@H](Cc1ccc(O)cc1)C(=O)OC]

To a solution of trans-2-octenoic acid (500 mg, 3.52 mmol) in DCM (3.5 mL) was added subsequently DIPEA (1.5 mL, 1.14 g, 8.79 mmol) and HBTU (1.60 g, 4.22 mmol). The mixture was stirred for 5 min at room temperature (r.t.) followed by the addition of H-Tyr-OMe HCl (896 mg, 3.87 mmol). The reaction was stirred at r.t. overnight. During this time the colourless suspension became a yellowish solution. The reaction was quenched by adding 10% aq. citric acid solution to the reaction mixture. The suspension was extracted three times with DCM. The combined organic layers were washed with sat. NaHCO₃ and brine, dried over MgSO₄, filtered and evaporated. The crude product was purified by flash chromatography over a silica gel column (mobile phase: cyclohexane/EtOAc 1:0 to 1:1). The appropriate fractions were collected and evaporated to afford ester A (878 mg, 2.75 mmol, 78% yield) as yellowish oil.

¹H-NMR (600 MHz, CDCl₃): δ=6.94-6.93 (m, 2H, 5-H), 6.85 (dt, J=15.3, 6.9 Hz, 1H, 10-H), 6.74-6.72 (m, 2H, 6-H), 5.96 (d, J=7.9 Hz, 1H, NH), 5.77 (d*, J=15.3 Hz, 1H, 9-H, *fine splitting), 4.93 (dt, J=7.9, 5.7 Hz, 1H, 2-H), 3.73 (s, 3H, OMe), 3.17 (dd, J=14.0, 5.7 Hz, 1H, 3-H), 3.04 (dd, J=14.0, 5.7 Hz, 1H, 3-H), 2.16 (q*, J=6.9 Hz, 2H, 11-H, *fine splitting), 1.43 (quint, J=7.3 Hz, 2H, 12-H), 1.32-1.25 (m, 4H, 13-H, 14-H), 0.88 (t, J=7.0 Hz, 3H, 15-H) ppm.

¹³C-NMR (150 MHz, CDCl₃): δ=172.3, 165.7, 155.2 (3s, C-1, C-8, C-7), 146.4 (d, C-10), 130.3 (d, 2×C-5), 127.3 (s, C-4), 122.7 (d, C-9), 115.5 (d, 2×C-6), 53.3 (d, C-2), 52.4 (q, OMe), 37.2, 32.0, 31.3, 27.8, 22.4 (5t, C-3, C-11, C-13, C-12, C-14), 14.0 (q, C-15) ppm.

HRMS (ESI-TOF): calculated for C₁₈H₂₆NO₄ [M+H]⁺: 320.1856; found 320.1859.

### 2.2. (E)-Oct-2-enoyl-tyrosine (B)

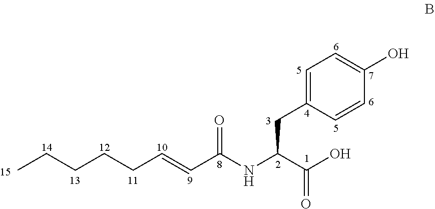 [SMILES: CCCCC/C=C/C(=O)N[C@@H](Cc1ccc(O)cc1)C(=O)O]

added 10% aq. KOH (250 μl) and the mixture was stirred for 3 h at r.t. The reaction was acidified by adding 1M HCl and then extracted with EtOAc twice. The combined organic layers were washed with water and brine, dried over MgSO₄, filtered and evaporated to afford acid B (79 mg, crude product) as clear oil. The crude product was used in the next step without further purification.

¹H-NMR (600 MHz, MeOD-d₄): δ=7.04-7.02 (m, 2H, 5-H), 6.74 (dt, J=15.4, 7.1 Hz, 1H, 10-H), 6.69-6.68 (m, 2H, 6-H), 5.95 (d*, J=15.4 Hz, 1H, 9-H, *fine splitting), 4.65 (dd, J=8.7, 5.2 Hz, 1H, 2-H), 3.11 (dd, J=14.0, 5.2 Hz, 1H, 3-H), 2.89 (dd, J=14.0, 8.7 Hz, 1H, 3-H), 2.18 (q*, J=7.0 Hz, 2H, 11-H, *fine splitting), 1.45 (quint, J=7.3 Hz, 2H, 12-H), 1.36-1.29 (m, 4H, 13-H, 14-H), 0.91 (t, J=7.0 Hz, 3H, 15-H) ppm.

¹³C-NMR (150 MHz, MeOD-d₆): δ=174.9, 168.5, 157.3 (3s, C-1, C-8, C-7), 146.4 (d, C-10), 131.2 (d, 2×C-5), 129.1 (s, C-4), 124.2 (d, C-9), 116.2 (d, 2×C-6), 55.4 (d, C-2), 37.7, 33.0 (t, C-11), 32.5, 29.1, 23.5 (5t, C-3, C-11, C-13, C-12, C-14), 14.3 (q, C-15) ppm.

HRMS (ESI-TOF): calculated for C₁₇H₂₄NO₄ [M+H]⁺: 306.1700; found 306.1702.

### 2.3. Fmoc-Arg-(Boc)₂-OH (C)

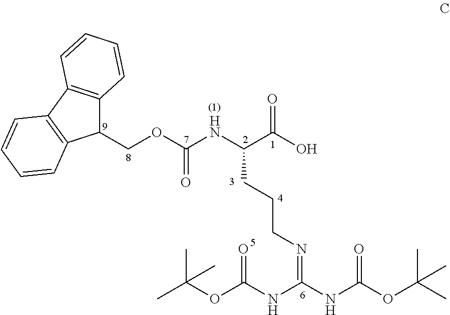 [SMILES: CC(C)(C)OC(=O)NC(=NCCC[C@H](NC(=O)OCC1c2ccccc2-c2ccccc21)C(=O)O)NC(=O)OC(C)(C)C]

mmol) and basic copper carbonate CuCO₃ (656 mg, 2.97 mmol) dissolved in dest. H₂O (8 mL) was stirred under reflux for 15 min. The blue reaction mixture was filtered, evaporated and dried under vacuum to afford a blue solid (1.22 g, quant).

Part of the blue solid (600 mg) was suspended in formamide (6 mL) and DIPEA (1.2 mL, 880 mg, 6.81 mmol) was added. To the blue solution was added drop wise bis-boc-pyrazolocarboxamidine (939 mg, 3.02 mmol) in dioxane (3 mL). The blue solution was stirred at r.t. overnight, and then quenched with H₂O (20 mL) to yield a light blue solid precipitate. The suspension was extracted twice with EtOAc. The combined organic layers were washed with brine, dried over MgSO₄, filtered and evaporated to yield blue foam (1.48 g). The crude product was suspended in H₂O (6 mL) and EDTA·2Na·2H₂O (676 mg, 1.82 mmol) and sat. aq. NaHCO₃ (597 mg, 7.11 mmol) were added, then 9-fluorenylmethyl-succinimidyl carbonate (1.22 g, 3.63 mmol), dissolved in acetone (13.5 mL), was added drop wise to the reaction mixture at r.t. The blue suspension turned light blue and the reaction mixture was stirred at r.t. overnight. Subsequently, the solvent was evaporated under vacuum, and H₂O (25 mL) was added resulting in a slight basic light blue turbid mixture (pH 8). The mixture was acidified by adding 10% aq. citric acid solution (pH 2-3). The resulting solution was extracted twice with EtOAc. The combined organic layers were washed with water and brine, dried over MgSO₄, filtered and evaporated. The crude product was purified by chromatography over a silica gel column (mobile phase: cyclohexane/EtOAc+1% AcOH 10:1 to 1:1). The appropriate fractions were collected and evaporated to afford triprotected arginine C (1.22 g, 2.05 mmol, 70% yield over two steps) as colorless foam.

¹H-NMR (600 MHz, CDCl₃): δ=11.37 (br. s, 1H, NHCO₂tBu), 8.50, 8.42* (s, 1H, NHCO₂tBu), 7.75-7.74 (m, 2H, Ar), 7.62-7.59, 7.56* (m, 2H, Ar), 7.39-7.37 (m, 2H, Ar), 7.31-7.28 (m, 2H, Ar), 5.89*, 5.83 (d, J=8.1 Hz, NH (1)), 4.62*, 4.52*, 4.42-4.37 (m, 3H, 2-H, 8-H), 4.21 (t, J=7.0 Hz, 1H, 9-H), 3.46-3.39, 3.32* (m, 2H, 5-H), 1.98-1.94 (m, 1H, 3-H), 1.76-1.66 (m, 3H, 3-H, 4-H), 1.48 (s, 9H, NHCO₂tBu), 1.47 (s, 9H, NHCO₂tBu) ppm *minor rotamer.

¹³C-NMR (150 MHz, CDCl₃): δ=174.9, 172.5* (s, C-1), 163.3*, 162.8 (s, C-6), 156.6*, 156.4 (s, C-7), 156.4 (s, NHCO₂tBu), 153.3, 152.9* (s, NHCO₂tBu), 144.0*, 143.8 (2s, Ar), 141.4 (s, 2Ar), 127.8 (d, 2Ar), 127.2 (d, 2Ar), 125.3, 124.9* (d, Ar), 120.1 (d, 2Ar), 84.3*, 83.7, 83.4* (2s, NHCO₂tBu), 80.5*, 80.1, 79.7* (2s, NHCO₂t̅B̅u̅), 67.3*, 67.1 (t, C-8), 54.1*, 53.7 (d, C-2), 47.3 (d, C-9), 40.6, 40.4* (t, C-5), 29.8*, 29.7 (t, C-3), 28.4*, 28.3 (q, NHCO₂tBu), 28.2*, 28.2 (q, NHCO₂tBu), 25.5, 25.3* (t, C-4) ppm *minor rotamer.

HRMS (ESI-TOF): calculated for C₃₁H₄₁N₄O₈ [M+H]⁺ 597.2919; found 597.2924.

### 2.4. N1-Fmoc-N3,N4-Di-Boc-Arginine Weinreb Amide (D)

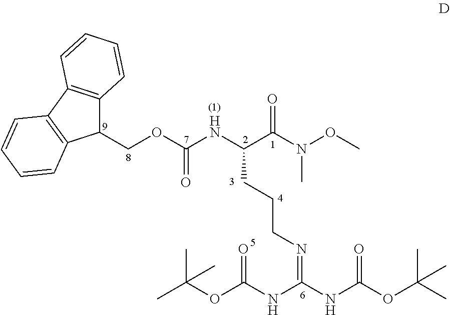 [SMILES: CON(C)C(=O)[C@H](CCCN=C(NC(=O)OC(C)(C)C)NC(=O)OC(C)(C)C)NC(=O)OCC1c2ccccc2-c2ccccc21]

added subsequently DIPEA (263 μl, 195 mg, 1.51 mmol) and HBTU (229 mg, 0.603 mmol). The mixture was stirred at r.t. for 5 min followed by the addition of N,O-dimethylhydroxylamine hydrochlorid (98 mg, 1.01 mmol). The reaction was stirred at r.t. overnight. To the reaction mixture was added H₂O and the resulting suspension was extracted twice with EtOAc. The combined organic layers were washed with brine, dried over Na₂SO₄, filtered and evaporated. The crude product was purified by flash chromatography over a silica column (mobile phase: cyclohexane/EtOAc 1:0 to 1:1). The appropriate fractions were collected and evaporated to afford weinreb amide D (228 mg, 0.356 mmol, 71% yield) as colorless foam.

¹H-NMR (500 MHz, CDCl₃): δ=11.50 (s, 1H, NHCO₂tBu), 8.40, 8.28* (s, 1H, NHCO₂tBu), 7.77-7.75 (m, 2H, Ar), 7.62-7.60, 7.57* (m, 2H, Ar), 7.41-7.38 (m, 2H, Ar), 7.33-7.30 (m, 2H, Ar), 5.61, 5.15* (d, J=8.9 Hz, 1H, NH(1)), 4.78-4.70, 4.60* (m, 1H, 2-H), 4.39 (dd, J=10.5, 7.5 Hz, 1H, 8-H), 4.35 (dd, J=10.5, 7.0 Hz, 1H, 8-H), 4.22 (t, J=7.0 Hz, 1H, 9-H), 3.77 (s, 3H, N(CH₃)OCH₃), 3.47-3.46, 3.36* (m, 2H, 5-H), 3.22, 3.11* (s, 3H, N(CH₃OCH₃), 1.84-1.80 (m, 1H, 3-H), 1.69-1.62 (m, 3H, 3-H, 4-H), 1.50 (s, 9H, NHCO₂t̅B̅u̅), 1.49 (s, 9H, NHCO₂tBu) ppm *minor rotamer.

¹³C-NMR (125 MHz, CDCl₃): δ=172.2, 163.1, 156.1 (3s, C-1, C-6, C-7), 156.0, 153.2 (2s, NHCO₂tBu), 143.9*, 143.8 (s, 2×Ar), 141.3*, 141.3 (s, 2Ar), 127.6 (d, 2×Ar), 127.0 (d, 2×Ar), 125.2*, 125.1 (d, 2×Ar), 119.9*, 119.9 (d, 2×Ar), 83.3, 79.5 (2s, NHCO₂tBu), 67.0 (t, C-8), 61.6 (q, N(CH₃)OCH₃), 50.7, 47.2 (2d, C-2, C-9), 40.5 (t, C-5), 32.1 (q, N(CH₃)OCH₃), 29.9 (t, C-3), 28.3, 28.0 (2q, NHCO₂tBu), 25.1 (t, C-4) ppm *minor rotamer.

HRMS (ESI-TOF): calculated for C₃₃H₄₆N₅O₈ [M+H]⁺ 640.3341; found 640.3347.

### 2.5. N1-Fmoc-N3,N4-di-Boc-vinylogous arginine ethyl ester (E)

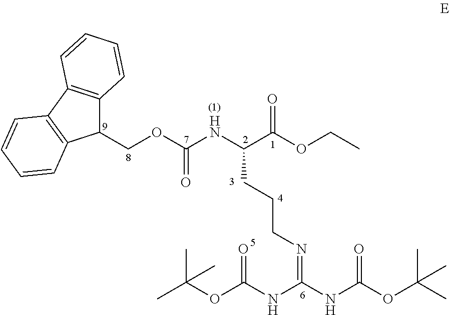 [SMILES: CCOC(=O)[C@H](CCCN=C(NC(=O)OC(C)(C)C)NC(=O)OC(C)(C)C)NC(=O)OCC1c2ccccc2-c2ccccc21]

A solution of weinrebamide D (221 mg, 0.345 mmol) in dry THF (3.5 mL) was cooled to 0° C. followed by slow addition of LiAlH₄ (66 mg, 1.73 mmol). The mixture was stirred for 30 min at 0° C. The reaction was quenched with 10% aq. citric acid solution and extracted twice with EtOAc. The combined organic layers were washed with water and brine, dried over MgSO₄, filtered. The solvent was removed under reduced pressure to afford the aldehyde (146 mg) as colorless foam which was used in the next step without further purification.

To an ice-cooled solution of triethyl phosphonoacetate (60 μl, 68 mg, 0.302 mmol) in dry THF (0.5 mL) was added NaH (15 mg, 0.377 mmol, 60% in mineral oil). After 30 min of stirring at 0° C., the crude aldehyde (146 mg) dissolved in dry THF (2 mL) was added drop wise at 0° C. to the reaction mixture. Upon completed addition, the reaction was stirred for 45 min at 0° C. The mixture was poured into 10% aq. citric acid solution and the suspension was extracted twice with EtOAc. The combined organic layers were washed with brine, dried over MgSO₄, filtered and evaporated. The crude product was purified by chromatography over a silica gel column (mobile phase: cyclohexane/EtOAc 5:1). The appropriate fractions were collected and evaporated to afford protected vinyl arginine E (71 mg, 0.109 mmol, 32% yield over two steps) as colorless oil.

¹H-NMR (300 MHz, CDCl₃): δ=11.52 (s, 1H, NHCO₂tBu), 8.39 (m, 1H, NHCO₂tBu), 7.76-7.73 (m, 2H, Ar), 7.66-7.59 (m, 2H, Ar), 7.40-7.35 (m, 2H, Ar), 7.32-7.25 (m, 2H, Ar), 6.84 (dd, J=15.6 Hz, 5.0 Hz, 1H, 3-H), 6.05 (d, J=8.9 Hz, 1H, NH(1)), 5.98 (d, J=15.6 Hz, 1H, 2-H), 4.44-4.41 (m, 3H, 4-H, 10-H), 4.22-4.14 (q, J=7.1 Hz, 3H, OCH₂CH₃, 11-H), 3.58-3.53 (m, 1H, 7-H), 3.32-3.26 (m, 1H, 7-H), 1.66-1.58 (m, 4H, 5-H, 6-H), 1.49 (s, 18H, 2 NHCO₂tBu), 1.28 (t, J=7.1 Hz, 3H, OCH₂CH₃) ppm.

¹³C-NMR (75 MHz, CDCl₃): δ=166.3, 163.5, 156.6 (3s, C-1, C-8, C-9), 156.0, 153.4 (2s, NHCO₂tBu), 148.0 (d, C-3), 144.1, 143.8, 141.4, 141.3 (4s, Ar), 127.7 (d, 2×Ar), 127.1 (d, 2×Ar), 125.3, 125.1 (2d, Ar), 121.1 (d, C-2), 120.0 (d, 2×Ar), 83.3, 79.5 (2s, NHCO₂tBu), 66.6 (t, C-10), 60.5 (t, OCH₂CH₃), 52.3, 47.4 (2d, C-4, C-11), 40.3 (t, C-7), 30.2, 29.7* (t, C-5), 28.3, 28.1 (2q, NHCO₂tBu), 27.0*, 26.2 (t, C-6), 14.3 (q, OCH₂CH₃) ppm *minor rotamer.

HRMS (ESI-TOF): calculated for C₃₅H₄₇N₄O₈ [M+H]⁺ 651.3388; found 651.3400.

### 2.6. N3,N4-Di-Boc-vinylogous arginine ethyl ester (F)

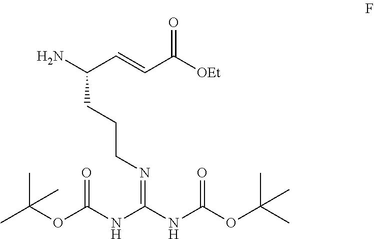 [SMILES: CCOC(=O)/C=C/[C@@H](N)CCCN=C(NC(=O)OC(C)(C)C)NC(=O)OC(C)(C)C]

Compound E (71 mg, 0.109 mmol) was dissolved in DMF (1 mL) and piperidine (200 μl) was added. The solution was stirred at r.t. for 4 h. The reaction was evaporated and dried under vacuum overnight. The crude product was purified by chromatography over a silica gel column (mobile phase gradient: from cyclohexane/EtOAc 1:1 to 0:1, switched to DCM/MeOH 9:1). The appropriate fractions were collected and evaporated to afford amine F (40 mg, 0.093 mmol, 86% yield) as colorless oil.

¹H-NMR (300 MHz, MeOD-d₄): δ=6.88 (dd, J=15.7 Hz, 6.7 Hz, 1H, 3-H), 5.98 (dd, J=15.7 Hz, 1.2 Hz, 1H, 2-H), 4.18 (q, J=7.1 Hz, 2H, OCH₂CH₃), 3.53-3.47 (m, 1H, 4-H), 3.40-3.35 (m, 2H, 7-H), 1.68-1.57 (m, 4H, 5-H, 6-H), 1.52 (s, 9H, NHCO₂tBu), 1.47 (s, 9H, NHCO₂tBu), 1.28 (t, J=7.1 Hz, 3H, OCH₂CH₃) ppm.

¹³C-NMR (75 MHz, MeOD-d₄): δ=168.1, 164.5 (2s, C-1, C-8), 157.6, 154.1 (2s, NCO₂tBu), 152.7, 121.5 (2d, C-3, C-2), 84.4, 80.3 (2s, NHCO₂tBu), 61.5 (t, OCH₂CH₃), 53.2 (d, C-4), 41.4, 34.5 (2t, C-7, C-5), 28.6, 28.2 (2q, NHCO₂tBu), 26.5 (t, C-6), 14.5 (q, OCH₂CH₃) ppm.

HRMS (ESI-TOF): calculated for C₂₀H₃₇N₄O₆ [M+H]⁺: 429.2708; found 429.2713.

### 2.7. (E)-Oct-2-enoyl-tyrosine-N3,N4-di-Boc-vinylogous arginine ethyl ester (G)

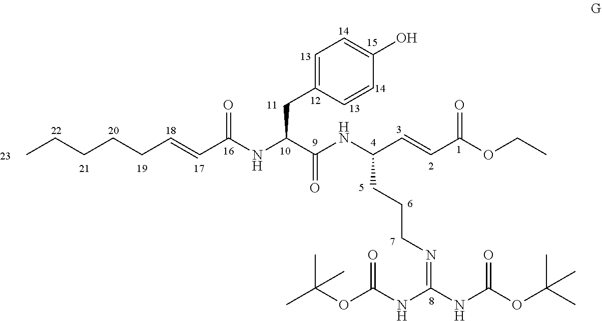 [SMILES: CCCCC/C=C/C(=O)N[C@@H](Cc1ccc(O)cc1)C(=O)N[C@H](/C=C/C(=O)OCC)CCCN=C(NC(=O)OC(C)(C)C)NC(=O)OC(C)(C)C]

To a solution of amine F (40 mg, 0.093 mmol) and octenoic acid (43 mg, 0.140 mmol) in DMF (1.0 mL) was added DIPEA (81 μl, 60 mg, 0.467 mmol) (pH>7). The mixture was cooled to 0° C. followed by the addition of COMU (64 mg, 0.149 mmol). The solution was stirred for 6 h at this temperature. The reaction was poured into 10% aq. citric acid solution and the suspension was extracted with EtOAc twice and three times with a DCM/IPA (4:1) mixture. The combined organic layers were washed with H₂O and brine, dried over Na₂SO₄, filtered and evaporated. The crude product was purified by HPLC using a semipreparative phenyl hexyl column (mobile phase: A: 20 mM aq. NH₄OAc, pH 7, B: MeOH gradient: 80% B 5 min, from 80% to 97% B in 30 min, 97% B 15 min). The appropriate fractions were collected and evaporated to afford compound G (18 mg, 0.025 mmol, 27% yield) as yellowish oil.

¹H-NMR (300 MHz, MeOD-d₄): δ=7.05-7.02 (m, 2H, 13-H), 6.82-6.66 (m, 4H, 3-H, 14-H, 18-H), 5.97 (dt, J=15.4 Hz, 1.5 Hz, 1H, 17-H), 5.64 (dd, J=15.7 Hz, 1.5 Hz, 1H, 2-H), 4.60-4.55 (m, 1H, 10-H), 4.52-4.47 (m, 1H, 4-H), 4.18 (q, J=7.0 Hz, 2H, OCH₂CH₃), 3.39-3.33 (m, 2H, 7-H), 2.97 (dd, J=13.5 Hz, 8.0 Hz, 1H, 11-H), 2.84 (dd, J=13.5 Hz, 7.3 Hz, 1H, 11-H), 2.18 (q*, J=7.1 Hz, 2H, 19-H, *fine splitting), 1.67-1.53 (m, 4H, 5-H, 6-H), 1.52 (s, 9H, NHCO₂tBu), 1.46 (s, 9H, NHCO₂tBu), 1.45-1.41 (m, 2H, 20-H), 1.36-1.25 (m, 4H, 21-H & 22-H), 1.29 (t, J=7.0 Hz, 3H, OCH₂CH₃), 0.91 (t, J=7.0 Hz, 3H, 23-H) ppm.

¹³C-NMR (75 MHz, MeOD-d₄): δ=173.4, 168.3, 167.9, 164.6 (4s, C-9, C-16, C-1, C-8), 157.6 (s, NHCO₂tBu), 157.4 (s, C-15), 154.1 (s, NHCO₂tBu), 148.8, 146.4 (2d, C-3, C-18), 131.3 (d, 2×C-13), 128.6 (s, C-12), 124.3, 122.0 (2d, C-17, C-2), 116.3 (d, 2×C-14), 84.4, 80.3 (2s, NHCO2tBu), 61.6 (t, OCH₂CH₃), 56.7, 51.2 (2d, C-10, C-4), 41.3, 38.5, 33.0, 32.5, 31.9, 29.1 (6t, C-7, C-11, C-19, C-21, C-5, C-20), 28.6, 28.3 (q, NHCO₂tBu), 26.7 (t, C-6), 23.5 (2q, NHCO2tBu), 14.6 (q, OCH₂CH₃), 14.4 (q, C-23) ppm.

### 2.8. (E)-Oct-2-enoyl-tyrosine-vinylogous arginine ethyl ester (3)

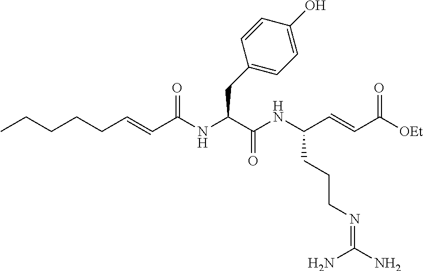 [SMILES: CCCCC/C=C/C(=O)N[C@@H](Cc1ccc(O)cc1)C(=O)N[C@H](/C=C/C(=O)OCC)CCCN=C(N)N]

A solution of ester G (18 mg, 25.1 μmol) in DCM (950 μl) was treated with TFA (50 μl) for 1 hour at 0° C. followed by stirring the reaction for 1.5 h at r.t. Additional TFA (50 μl) was added and the reaction was stirred at r.t. overnight. The reaction mixture was evaporated and dried under vacuum to afford the ester 3 as yellowish oil. The crude product was used in the next step without further purification.

¹H-NMR (500 MHz, MeOD-d4): δ=7.05-7.03 (m, 2H, 13-H), 6.76 (dt, J=15.4 Hz, 7.0 Hz, 1H, 18-H), 6.70-6.68 (m, 2H, 14-H), 6.66 (dd, J=15.8 Hz, 5.6 Hz, 1H, 3-H), 5.98 (d*, J=15.4 Hz, 1H, 17-H, *fine splitting), 5.59 (dd, J=15.8 Hz, 1.5 Hz, 1H, 2-H), 4.49 (m, 2H, 4-H, 10-H), 4.19 (dq, J=7.1 Hz, 2H, OCH₂CH₃), 3.21-3.11 (m, 2H, 7-H), 2.95 (dd, J=13.6 Hz, 8.3 Hz, 1H, 11-H), 2.87 (dd, J=13.6 Hz, 7.1 Hz, 1H, 11-H), 2.19 (q*, J=7.1 Hz, 2H, 19-H, *fine splitting), 1.70-1.58 (m, 3H, 5-H, 6-H), 1.57-1.52 (m, 1H, 5-H), 1.49-1.44 (m, 2H, 20-H), 1.37-1.31 (m, 4H, 21-H, 22-H), 1.29 (t, J=7.1 Hz, 3H, OCH₂CH₃), 0.91 (t, J=6.9 Hz, 3H, 23-H) ppm.

¹³C-NMR (125 MHz, MeOD-d4): δ=173.7, 168.5, 167.9, 158.7, 157.5 (5s, C-9, C-16, C-1, C-8, C15), 148.2, 146.4 (2d, C-3, C-18), 131.3 (d, 2×C-13), 128.5 (s, C-12), 124.3, 122.2 (2d, C17, C-2), 116.4 (d, 2×C-14), 61.7 (t, OCH₂CH₃), 57.1, 50.7 (2d, C-10, C-4), 42.0, 38.3, 33.0, 32.5, 32.0, 29.1, 26.1, 23.5 (8t, C-7, C-11, C-19, C-21, C-5, C-20, C-6, C-22), 14.5 (q, OCH₂CH₃), 14.3 (q, C-23) ppm.

HRMS (ESI-TOF): calculated for C₂₇H₄₁N₅O₅ [M+H]+: 516.3180; found 516.3183.

### 2.9. Barnesin A (1)

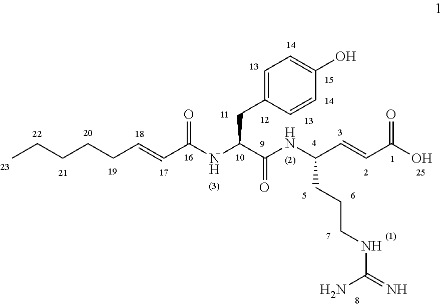 [SMILES: CCCCC/C=C/C(=O)N[C@@H](Cc1ccc(O)cc1)C(=O)N[C@H](/C=C/C(=O)O)CCCNC(=N)N]

Compound 3 was dissolved in DMSO (36 μl) and H₂O (144 μl) followed by the addition of porcine liver esterase (36 mg) in Tris/HCl buffer (1 mL, 50 mM, pH 6.8). The mixture was shaken for 5 days at 37° C. The reaction was filtered twice over a SPE cartridge. The methanolic and aqueous fractions were combined and evaporated. The crude product was purified by HPLC using a semipreparative phenyl hexyl column (mobile phase: A: 20 mM aq. NH₄OAc, pH 7, B: MeOH gradient: 30% B 5 min, from 30% to 80% B in 35 min, 80% B 10 min, from 80% to 95% B in 0.1 min, 95% B 4.9 min). The appropriate fractions were collected and evaporated to afford barnesin A (1) (7.0 mg, 14.4 μmol, 47% yield over two steps, colourless solid).

HRMS (ESI-TOF): calculated for C₂₅H₃₈N₅O₅ [M+H]⁺: 488.2867; found 488.2876.

[α]_{D}²⁵=−5.09° (0.0910; MeOH)

TABLE 1A
NMR data of barnesin A (1) (DMSO-d₆, at 300K)^{a}.

| position | δ_{H}, mult. (J in Hz) | δ_{C}, type^{b} | COSY | HMBC |
|---|---|---|---|---|
| 1 | | 169.7, qC | | |
| 2 | 5.73, d (15.4) | 124.7, CH | 3 | 1, 4 |
| 3 | 6.46, dd (15.7, 4.8) | 144.3, CH | 2, 4 | 1, 2, 4 |
| 4 | 4.34, m | 48.9, CH | 3, 5, NH(2) | |
| 5a | 1.46^{d}, m | 30.7, CH₂ | 4, 5b, 6 | |
| 5b | 1.69, m | | 4, 5a | 6 |
| 6 | 1.45, m | 24.9, CH₂ | 5a, 5b, 7 | 4, 5, 7 |
| 7 | 3.06, m | 40.4^{c}, CH₂ | 6 | 6, 8 |
| 8 | | 157.4, qC | | |
| 9 | | 171.1, qC | | |
| 10 | 4.47, m | 54.6, CH | 11a, 11b, NH(1) | 9, 11, 12, 16 |
| 11a | 2.87, m | 37.3, CH₂ | 10, 11b | 10, 12 |
| 11b | 2.59, m | | 10, 11a | 10, 12 |
| 12 | | 128.0, qC | | |
| 13 | 7.00, d (8.3) | 129.9, CH | 14 | 11, 13, 14, 15 |
| 14 | 6.60, d (8.2) | 114.8, CH | 13 | 13, 14, 15 |
| 15 | | 155.7, qC | | |
| 16 | | 164.7, qC | | |
| 17 | 5.90, d (15.3) | 124.3, CH | 18, 19 | 16 |
| 18 | 6.50, dt (15.4, 6.8) | 142.6, CH | 17, 19 | 16, 17, 19 |
| 19 | 2.08, m | 31.1, CH₂ | 18, 20 | 17, 18, 20, 21 |
| 20 | 1.35, m | 27.5, CH₂ | 19 | 18, 20, 21 |
| 21 | 1.27, m | 30.8, CH₂ | | 20, 22, 23 |
| 22 | 1.23, m | 21.9, CH₂ | 23 | 20, 21, 23 |
| 23 | 0.85, t (6.7) | 13.9, CH₃ | 22 | 21, 22 |
| NH(3) | 9.80, br. S | | 7 | |
| NH(2) | 8.46, d (7.9) | | 4 | 9 |
| NH(1) | 8.05, d (8.5) | | 10 | 10, 11, 16 |

^{a}600 MHz for ¹H NMR, COSY, HSQC and HMBC; 150 MHz for ¹³C NMR;
^{b}numbers of attached protons were determined by analysis of 2D spectra;
^{c}to be seen in HSQC;
^{d}to be seen in COSY.

### Example 3 Synthesis According to Reaction Scheme 2

### 3.1. Preparation of Aldehyde

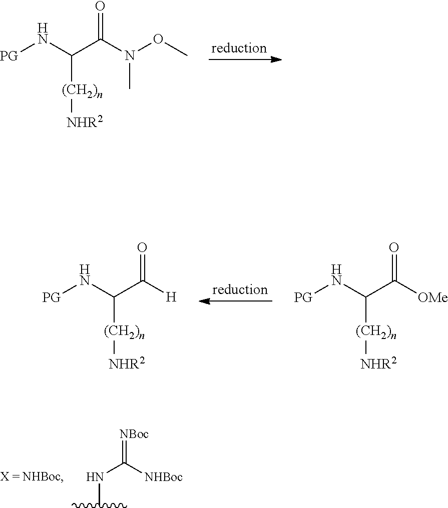

Weinreb amide route (GPC): Weinreb amide was solubilized in dry THF (1.0 mmol/mL) and cooled to 0° C. LiAlH₄ (5.0 eq) was added in portions over 30 min and the suspension was stirred at 0° C. for additional 2 h. Reaction control was performed using TLC. In case reduction was incomplete after 2 h, the reaction mixture was warmed up to r.t., LiAlH₄ (5.0-7.0 eq) was added and the reaction mixture was stirred for further 1-2 h depending on the reaction process. The reaction mixture was quenched using 10% aq. citric acid and extracted twice with EtOAc. The combined organic layers were washed twice with dest. H₂O, twice with brine, dried over MgSO₄, filtered and concentrated to obtain a yellow foamy oil. The crude product was used without further purification in the Horner-Wasdsworth-Emmons (HWE) reaction (see below).

Methyl ester route (GPD): Amino acid methyl ester was solubilized in dry DCM (50 μmol/mL) and cooled to −80° C. DIBAL-(H) (1.2 M in toluene, 2.0 eq) was added dropwise over 1 h and the reaction mixture stirred at −80° C. for one additional hour. Reaction control was performed using TLC. In case reduction was incomplete after one hour, additional 0.5 eq DIBAL-(H) was added every 30 min until full conversion of the methyl ester to the aldehyde. The solution was then quenched with a sat. solution of Rochelle salt until the production of gas ceased. The reaction mixture was extracted with DCM and warmed up at r.t. DCM and dest. H₂O were added in order to get a clear suspension, which was strongly stirred for an additional hour. The aq. phase was at last extracted with DCM, and the combined org. layers were washed with brine, dried over Na₂SO₄, filtered and concentrated to obtain a yellowish oil. The crude product was used without further purification in the HWE-step (see below).

### 3.1.1 Products

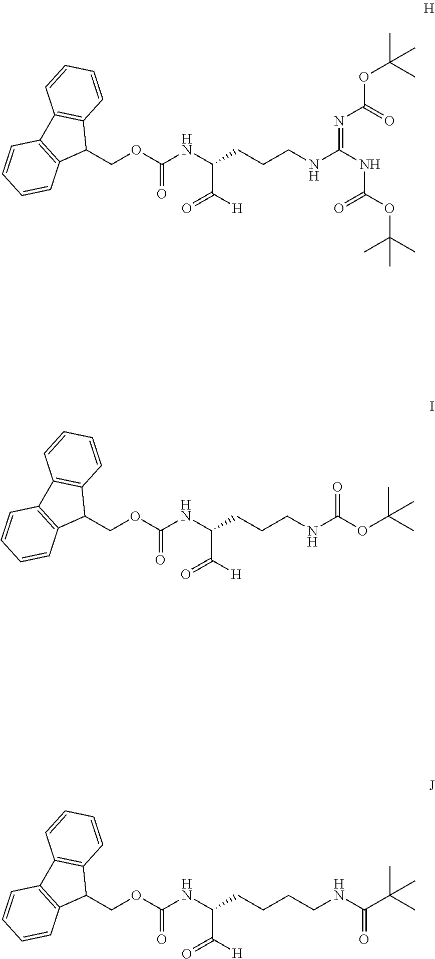 [SMILES: CC(C)(C)OC(=O)/N=C(/NCCC[C@H](C=O)NC(=O)OCC1c2ccccc2-c2ccccc21)NC(=O)OC(C)(C)C ; CC(C)(C)OC(=O)NCCC[C@H](C=O)NC(=O)OCC1c2ccccc2-c2ccccc21 ; CC(C)(C)C(=O)NCCCC[C@H](C=O)NC(=O)OCC1c2ccccc2-c2ccccc21]

Fmoc-Arg(ω,ω′-Boc) aldehyde (H): According to GPC, Fmoc-Arg(ω,ω′-Boc) Weinreb amide (110 mg, 0.17 mmol, 1.0 eq) was converted to aldehyde (H, 56 mg) as yellowish oil. According to GPD, Fmoc-L-Arg(ω,ω′-Boc)-OMe (720 mg, 1.18 mmol, 1.0 eq) was converted to aldehyde (H, 830 mg).

Fmoc-L-Orn(δ-Boc) aldehyde (I): According to GPD, Fmoc-Orn(δ-Boc)-OMe (340 mg, 0.73 mmol, 1.0 eq) was converted to aldehyde (I, 350 mg) as yellowish oil.

Fmoc-L-Lys(c-Boc) aldehyde (J): According to GPC, Fmoc-Lys(c-Boc) Weinreb amide (186 mg, 0.36 mmol, 1.0 eq) was converted to aldehyde (J. 126 mg) as yellowish oil. According to GPD, Fmoc-Lys(c-Boc)-OMe (340 mg, 0.70 mmol, 1.0 eq) was converted to aldehyde (J, 296 mg) as yellowish oil.

### 3.2. Solide Phase Peptide Synthesis (SPPS)

### 3.2.1 Loading of Resin

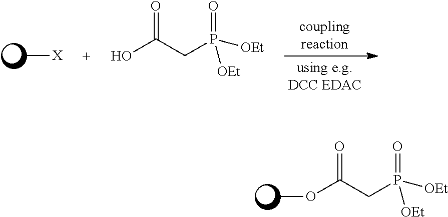

Wang Resin: Wang resin (275 mg, 1.40 mmol/g, 0.39 mmol, 1.0 eq) was activated by shaking in DMF (4 mL) for 1 h. The resin was filtrated, cooled to 0° C. and a solution of coupling reagents and diethylphosphonoacetic acid (377 mg, 1.92 mmol, 5.1 eq), dissolved in DMF (2 mL), was added at 0° C. The coupling reagents were either HBTU (719 mg, 1.93 mmol, 5.0 equ)/HOBt (265 mg, 1.94 mmol, 5.0 eq), or PyBOP (927 mg, 1.92 mmol, 5.1 eq). DIPEA (667 μL, 3.81 mmol, 10.0 eq) was added at the end. The resin was first shaken at 0° C. for 5 min, then at r.t. for additional 24 h. The reaction mixture turned red overtime. Lastly, the resin was filtrated and washed 3 times, alternatively, with DMF (2 mL), and IPA (2 mL). The last wash was with DMF. A capping step was added by shaking the resin a 20% acetanhydrid solution in DMF (2 mL) during 30 min. The resin was then again washed as previously described.

CTT Resin: CTT resin (300 mg, 0.875 mmol/g, 0.26 mmol, 1.0 eq) was swelled 1 h, while shaken, in dry DCM (4 mL). A solution of diethylposponoacetic acid (87 mg, 0.44 mmol, 2.5 eq), dissolved in dry DCM (2 mL), was poured on the resin with a first addition of DIPEA (61.1 μL, 0.35 mmol, 2.0 eq). After 5 minutes of shaking, at room temperature, DIPEA (91.7 μL, 0.53 mmol, 3.0 eq) was added a second time. The resin was them shaken at room temperature, overnight. The reaction mixture got yellow overtime. Lastly, the resin was capped by adding MeOH (300 μL), and shaken for further 30 minutes. The resin was filtrated and subsequently washed with DCM (2 mL), DMF (2 mL), DCM (2 mL) and, at the end with MeOH (2 mL).

### 3.2.2. Horner-Wasdsworth-Emmons Reaction on Solid Phase (GPE)

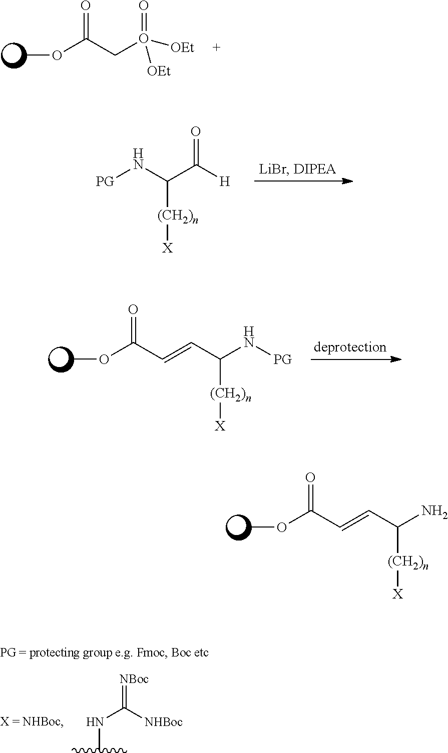

The amount of reagents was calculated estimating a resin loading of 0.50 mmol/g. The resin (300 mg, 0.50 mmol*g⁻¹, 0.15 mmol, 1.0 eq) was activated by shaking in dry THF (4 mL) for 1 h. A solution of LiBr (26.1 mg, 0.30 mmol, 2.0 eq) and DIPEA (41.1 μL, 0.23 mmol, 1.5 eq), solubilized in dry THF (1 mL), was poured on the resin, followed by the addition of the aldehyde (Table 2), also solubilized in dry THF (1 mL). The resin was shaken for 24 h at r.t.

TABLE 2
Amount of α-amino aldehydes.

| Aldehyde | Weight [mg] | n [mmole] | eq |
|---|---|---|---|
| Fmoc-L-Arg (ω,ω′-Boc)-H (H) | 261.3 | 0.45 | 3.0 |
| Fmoc-L-Orn (δ-Boc)-H (I) | 197.3 | 0.45 | 3.0 |
| Fmoc-L-Lys (ε-Boc)-H (J) | 206.8 | 0.46 | 3.1 |

Upon completed reaction, the resin was alternately washed three times with THF (2 mL) and IPA (2 mL). To determine the conversion during HWE reaction, the N-terminus was deprotected by shaking the resin three times with a 20% Piperidine DMF solution (1 mL) for 3 min. Piperidine was removed from the resin by washing alternately the resin three times with DMF (2 mL) and IPA (2 mL), and lastly with DMF (2 mL). An analytical amount of resin was then taken and analyzed using Kaiser test. In case of a positive Kaiser test, the peptide coupling step was performed (see GPF).

### 3.2.3. General Procedure for Peptide Coupling (GPF)

The general procedure F for 300 mg resin, with an estimated loading of 0.50 mmol/g, is summed up in

Table 3. All steps were carried out at room temperature. The coupling step was monitored using the Kaiser test (an analytical amount of the resin was taken, washed with DMF and analysed).

TABLE 3
Procedure for solid phase peptide synthesis.

| | | | Volume | Repeats × |
|---|---|---|---|---|
| Step | Operation | Reagents/Solvent | [mL] | time [min] |
| 1 | Coupling | Fmoc-Tyr(Bu)-OH, or fatty | 3 | 1 × 30 |
| | | acid (0.45 mmol, 3.0 eq), | | |
| | | HOBt (50.7 mg, 0.38 mmol, | | |
| | | 2.5 eq), | | |
| | | HBTU (142.2 mg, 0.38 mmol, | | |
| | | 2.5 eq), | | |
| | | DIPEA (137.0 μL, 0.75 mmol, | | |
| | | 5.0 eq) | | |
| | | in DMF | | |
| 2 | Washing | Alternating, DMF and IPA | 2 | 3 × 1 |
| 3 | Deprotection | 20% piperidine in DMF | 1 | 3 × 3 |
| 4^{b} | Washing | Alternating, DMF and IPA | 2 | 3 × 1 |

### 3.2.4. Cleavage of Protected Product from Resin

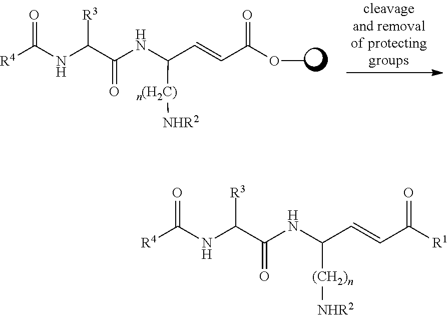

Wang Resin: The resin was shaken in 95% TFA, 2.5% TES and 2.5% H₂O (1 mL pro 100 mg resin) for 24 h at r.t. in order to get the unprotected peptide.

CTT Resin: The resin was shaken in HFIP:DCM (1:4) (1 mL pro 100 mg resin) for 15 min at r.t. in order to get the protected peptide.

Depending on the quantity of crude product, the peptide was either purified by semi-preparative HPLC (see methods above) or by reverse-phase flash chromatography.

### 3.2.5. Products

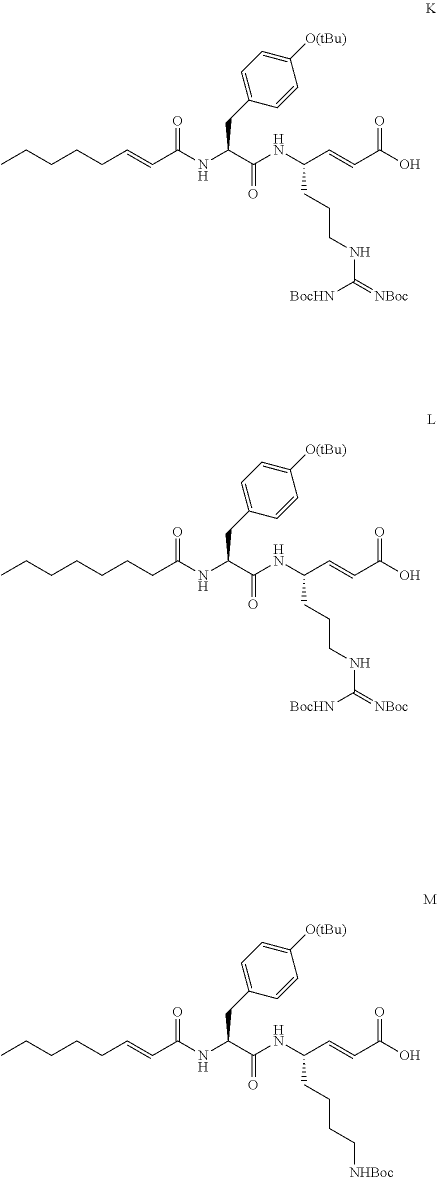

Protected barnesin (K): HRMS (ESI-TOF): calculated for C₃₉H₆₂O₉N₅ [M+H]⁺ 744.4542; found 744.4537.

Protected 17,18-Dihydrobarnesin (L): HRMS (ESI-TOF): calculated for C₃₉H₆₄O₉N₅ [M+H]⁺ 746.4699; found 744.4701.

Protected Lysin-Barnesin (M): HRMS (ESI-TOF): calculated for C₃₄H₅₄O₇N3 [M+H]+616.3956; found 616.3955.

### 3.3 Modification of Protected Lipodipeptides

### 3.3.1 Synthesis of Protected Barnesin Weinreb Amide (T)

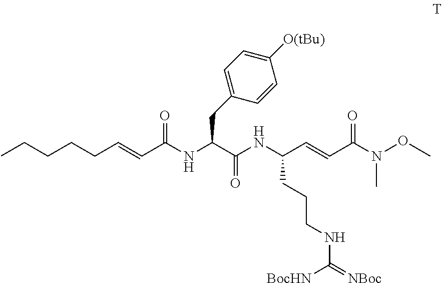

Protected barnesin (K, 4.5 mg, 6.0 μmol, 1.0 eq) was solubilized in DCM (2 mL) and cooled to 0° C. and then treated with HBTU (1.2 eq) and DiEPA (2.1 eq). Then hydrochloride salt of N,O-dimethylhydroxylamine (2.0 eq.) was added. The progress of the reaction was monitored by TLC. The reaction was quenched by aqueous work-up (brine, sodium carbonate solution) and the organic layer was dried over Na₂SO₄ and evaporated under reduced pressure. The pure Boc-Weinreb amide was isolated after column chromatography as colourless oil.

Protected barnesin weinreb amide (T): HRMS (ESI-TOF): calculated for C₄₁H₆₇N₆O₉ [M+H]⁺ 787.4970; found 787.4969.

### 3.3.2 Modifications of C-terminus (GPG)

The protected purified peptide was solubilized in dry MeOH (2 mL) and cooled to 0° C. TMS-diazomethane (5.0 eq) was added dropwise. A production of gas (N2) was observed and the solution turned yellow. The reaction mixture was stirred at r.t. The reaction was monitored by UHPLC-MS and in case of uncomplete conversion additional TMS-diazomethane (5.0 eq) was added every hour until full conversion of the peptide into the methyl ester. The reaction mixture was concentrated to obtain a yellowish oil, which was purified by semi preparative HPLC.

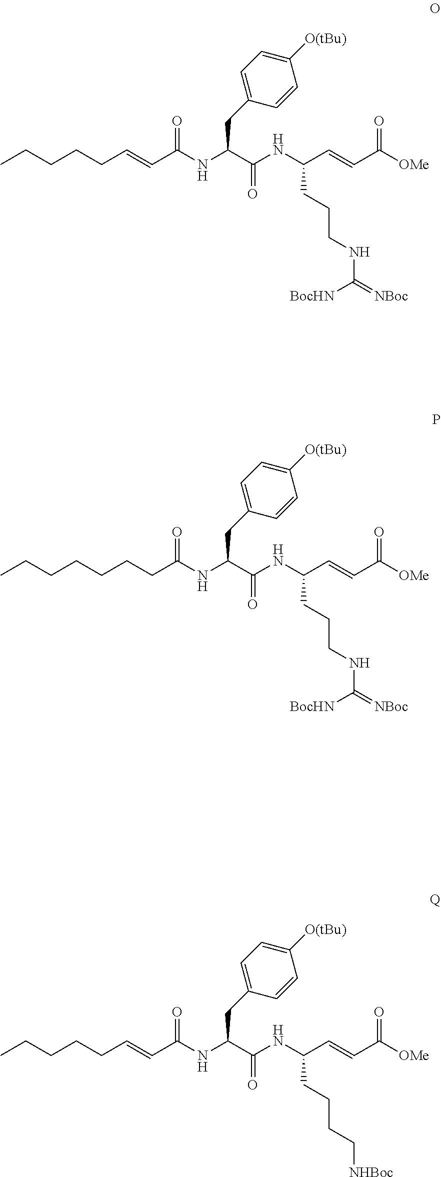

Protected barnesin methyl ester (O): According to GPG, protected peptide (K, 4.5 mg, 6.0 μmol 1.0 eq) was converted to ester (O, 2.5 mg after purification, 3.3 μmol 53% yield, colourless oil). HRMS (ESI-TOF): calculated for C₄₀H₆₄O₉N₅ [M+H]⁺ 758.4699; found 758.4694.

Protected 17,18-dehydrobarnesin methyl ester (P): According to GPG, protected peptide (L, 5.6 mg, 7.5 μmol 1.0 eq) was converted to ester (P) (2.7 mg after purification, 3.6 μmol 47% yield, colourless oil). HRMS (ESI-TOF): calculated for C₄₀H₆₆O₉N5 [M+I-1]+760.4855; found 758.4860.

Protected lysin-barnesin methyl ester (Q): According to GPG, protected peptide (M, 6.7 mg, 10.5 μmol 1.0 eq) was converted to ester (Q) (2.6 mg after purification, 4.1 μmol 38% yield, colourless oil). HRMS (ESI-TOF): calculated for C₃₅H₅₆O₇N₃ [M+H]⁺ 630.4113; found 630.4114.

### 3.3.3 Deprotection (GPH)

The protected purified peptide was cooled to 0° C. A solution of 95% TFA in DCM (1 mL) was poured on the peptide and the mixture was stirred and allowed to warm to r.t. over 24 h. Upon completed reaction, the mixture was concentrated to obtain the deprotected peptide as yellowish oil. It was purified on semi-preparative HPLC.

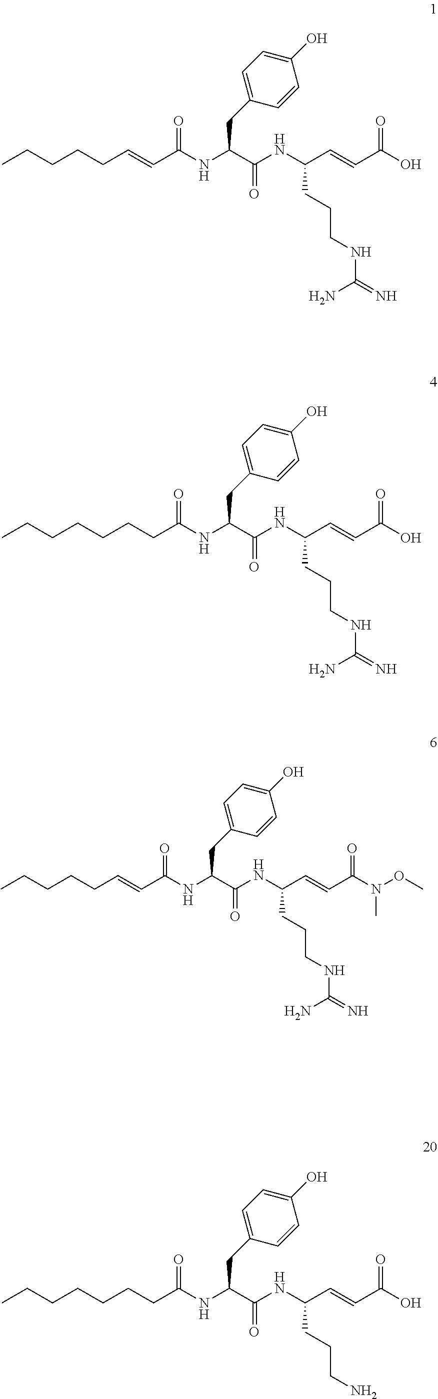

Barnesin A (1): According to GPH, protected barnesin (0, 4.4 mg, 5.9 μmol, 1.0 eq) was converted to barnesin (1) (1.5 mg, 3.1 μmol, 52% yield, colourless oil). HRMS (ESI-TOF): calculated for C₂₅H₃₈O₅N₅ [M+H]⁺ 488.2867; found 488.2871.

17,18-Dihydrobarnesin (4): According to GPH, protected purified 17,18-dihydrobarnesin (P, 5.6 mg, 7.5 μmol, 1.0 eq) was converted to 17,18-dihydrobarnesin (4, 3.5 mg, 7.1 μmol, 95% yield, colourless oil). HRMS (ESI-TOF): calculated for C₂₅H₄₀O₅N₅ [M+H]⁺ 490.3024; found 490.3020. IR (ATR) vₘₐₓ: 3273, 2956, 2927, 2856, 1632, 1541, 1516, 1457, 1393. [α]_{D}²⁵: −9.0° (c 1.0; MeOH). Weinreb barnesin (6): According to GPH, protected purified weinreb barnesin (T, 6 mg, 7.6 μmol, 1.0 eq.) was converted to Weinreb barnesin (6, 3 mg, 5.6 μmol, 73%, colourless oil). HRMS (ESI-TOF): calculated for C₂₇H₄₄N₆O₅ [M+H]⁺ 533.3451; found 533.3453.

Lysin-barnesin (20): According to GPH, protected purified lysin-barnesin (Q, 4.5 mg, 7.3 μmol, 1.0 eq) was converted to lysin-barnesin (20, 2.5 mg, 5.4 μmol, 74% yield, colourless oil). HRMS (ESI-TOF): calculated for C₂₅H₃₈O₅N₃ [M+H]⁺ 460.2806; found 460.2801.

### 3.3.4 Stereoisomers

Stereoisomers were synthesized according to the procedures outlined above using appropriate isomeric building blocks as starting materials. Final purification of the crude products was carried out on a Shimadzu HPLC system using a Phenomenex Luna C18 (2) 250×10.

a) Barnesin-D-Arginine-L-Tyrosine (1A)

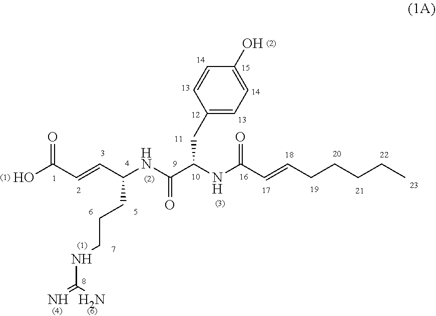 [SMILES: CCCCC/C=C/C(=O)N[C@@H](Cc1ccc(O)cc1)C(=O)N[C@@H](/C=C/C(=O)O)CCCNC(=N)N]

TABLE 1B
NMR data of Barnesin-D-Arginine-L-Tyrosine (1A)
(DMSO-d₆, at 300K)^{a}.

| position | δ_{H}, mult. (J in Hz) | δ_{C}, type^{b} | COSY | HMBC |
|---|---|---|---|---|
| 1 | | | | |
| 2 | 5.70, d (15.5) | 128.7, CH | 2 | 1, 4 |
| 3 | 6.37, dd (15.9, 5.3) | 141.2, CH | 2, 4 | 1, 2, 4 |
| 4 | 4.29, m | 48.7, CH | NH(2) | |
| 5a | 1.58^{d}, m | 30.9, CH₂ | 5b, 6 | |
| 5b | 1.45, m | | 5a, 6 | 6 |
| 6 | 1.29, m | 22.1, CH₂ | 5a, 5b, 7 | 4, 5, 7 |
| 7 | 2.97, m | 40.4^{c}, CH₂ | 6 | 6, 8 |
| 8 | | | | |
| 9 | | 170.8, qC | | |
| 10 | 4.52, m | 54.6, CH | 11a, 11b, NH(1) | 9, 11, 12, 16 |
| 11a | 2.82, m | 37.5, CH₂ | 10, 11b | 10, 12 |
| 11b | 2.66, m | | 10, 11a | 10, 12 |
| 12 | | 128.0, qC | | |
| 13 | 7.00, d (8.4) | 130.0, CH | 14 | 11, 13, 14, 15 |
| 14 | 6.60, d (8.4) | 114.9, CH | 13 | 13, 14, 15 |
| 15 | | 155.9, qC | | |
| 16 | | 164.6, qC | | |
| 17 | 5.93, d (15.5) | 124.4, CH | 18, 19 | 16 |
| 18 | 6.50, dt (15.1, 7.1) | 142.7, CH | 17, 19 | 16, 17, 19 |
| 19 | 2.07, m | 31.2, CH₂ | 18, 20 | 17, 18, 20, 21 |
| 20 | 1.35, m | 27.5, CH₂ | 19 | 18, 20, 21 |
| 21 | 1.26, m | 30.8, CH₂ | | 20, 22, 23 |
| 22 | 1.23, m | 21.9, CH₂ | 23 | 20, 21, 23 |
| 23 | 0.85, t (6.9) | 13.9, CH₃ | 22 | 21, 22 |
| NH(3) | 10.02, br. S | | 7 | |
| NH(2) | 8.21, d (8.3) | | 4 | 9 |
| NH(1) | 8.15, d (8.3) | | 10 | 10, 11, 16 |

^{a}600 MHz for ¹H NMR, COSY, HSQC and HMBC; 150 MHz for ¹³C NMR;
^{b}numbers of attached protons were determined by analysis of 2D spectra;
^{c}to be seen in HSQC;
^{d}to be seen in COSY.

b) Barnesin-L-Arginine-D-Tyrosine (1B)

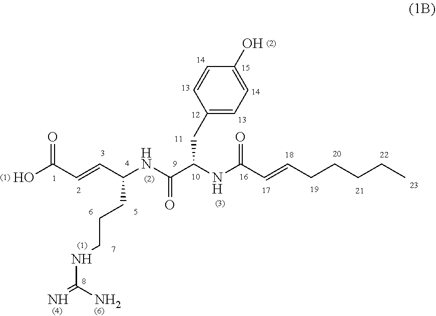 [SMILES: CCCCC/C=C/C(=O)N[C@@H](Cc1ccc(O)cc1)C(=O)N[C@@H](/C=C/C(=O)O)CCCNC(=N)N]

TABLE 1C
NMR data of Barnesin-L-Arginine-D-Tyrosine (1B)
(DMSO-d₆, at 300K)^{a}.

| position | δ_{H}, mult. (J in Hz) | δ_{C}, type^{b} | COSY | HMBC |
|---|---|---|---|---|
| 1 | | | | |
| 2 | 5.70, d (15.8) | 129.2, CH | 3 | 4 |
| 3 | 6.36, dd (15.8, 5.2) | 140.8, CH | 2, 4 | 1, 4 |
| 4 | 4.28, m | 49.2, CH | 3, 6, NH(2) | |
| 5a | 1.58, m | 29.1, CH₂ | 5b, 6 | |
| 5b | 1.45, m | | 5a, 6 | |
| 6 | 1.29, m | 22.5, CH₂ | 5a, 5b, 7 | 5, 7 |
| 7 | 2.97, m | 40.4^{c}, CH₂ | 6 | |
| 8 | | | | |
| 9 | | 171.2, qC | | |
| 10 | 4.52, m | 55.0, CH | 11a, 11b, NH(1) | |
| 11a | 2.82, m | 38.0, CH₂ | 10, 11b | 12, 13 |
| 11b | 2.66, m | | 10, 11a | 12, 13 |
| 12 | | 128.3, qC | | |
| 13 | 6.99, d (8.3) | 130.5, CH | 14 | 11, 13, 15 |
| 14 | 6.60, d (8.5) | 115.4, CH | 13 | 12, 14, 15 |
| 15 | | 156.3, qC | | |
| 16 | | 165.0, qC | | |
| 17 | 5.94, d (15.8) | 124.8, CH | 18, 19 | 16, 19 |
| 18 | 6.53, dt (15.0, 7.2) | 143.1, CH | 17, 19 | 16, 19, 20 |
| 19 | 2.07, m | 31.6, CH₂ | 18, 20 | 17, 18, 20, 21 |
| 20 | 1.35, m | 27.9, CH₂ | 19 | 18, 21, 22 |
| 21 | 1.24, m | 31.3, CH₂ | | 20, 22, 23 |
| 22 | 1.23, m | 22.3, CH₂ | 23 | 20, 21, 23 |
| 23 | 0.85, t (6.7) | 14.3, CH₃ | 22 | 21, 22 |
| NH(3) | 9.99, br. S | | | |
| NH(2) | 8.21, d (8.1) | | 4 | 9 |
| NH(1) | 8.15, d (8.1) | | 10 | 16 |

^{a}600 MHz for ¹H NMR, COSY, HSQC and HMBC; 150 MHz for ¹³C NMR;
^{b}numbers ofattached protons were determined by analysis of 2D spectra;
^{c}to be seein in HSQC;
^{d}to be seen in COSY.

c) Barnesin-D-Lysin (20A)

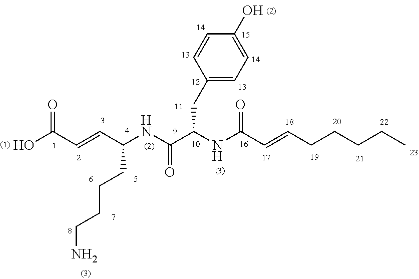 [SMILES: CCCCC/C=C/C(=O)N[C@@H](Cc1ccc(O)cc1)C(=O)N[C@@H](/C=C/C(=O)O)CCCCN]

TABLE 1D
NMR data of Barnesin-D-Lysin (20A) (DMSO-d₆, at 300K)^{a}.

| position | δ_{H}, mult. (J in Hz) | δ_{C}, type^{b} | COSY | HMBC |
|---|---|---|---|---|
| 1 | | 167.1, qC | | |
| 2 | 5.73, dd (15.7, 1.5) | 121.2, CH | 3 | 1, 4 |
| 3 | 6.70, dd (15.7, 5.5) | 147.9, CH | 2, 4 | 1, 2, 4, 5 |
| 4 | 4.39, m^{c} | 49.1, CH | 5a, 5b, | 2, 3, 5 |
| | | | NH(2) | |
| 5a | 1.57, m^{c} | 32.8, CH₂ | 4 | 4, 6 |
| 5b | 1.44, m^{c} | | 4 | 4, 6 |
| 6 | 1.27, m^{c} | 22.2, CH₂ | | |
| 7 | 1.51, m | 36.6, CH₂ | 8 | 6, 8 |
| 8 | 2.75, t (7.57) | 38.7, CH₂ | 7 | 6, 7 |
| 9 | | 171.1, qC | | |
| 10 | 4.45, m | 54.6, CH | 11a, 11b, | 8, 9, 11 |
| | | | NH(1) | |
| 11a | 2.85, m^{c} | 37.1, CH₂ | 10, 11b | 9, 10, 12, 13 |
| 11b | 2.67, m^{c} | | 10, 11a | 9, 10, 12, 13 |
| 12 | | 127.8, qC | | |
| 13 | 7.03, d (8.3)^{c} | 130.0, CH | 14 | 11, 13, 14, 15 |
| 14 | 6.62, d (8.3)^{c} | 114.9, CH | 13 | 12, , 13, 14, 15 |
| 15 | | 155.8, qC | | |
| 16 | | 164.8, qC | | |
| 17 | 5.95, d (15.4)^{c} | 124.2, CH | 18 | 16, 19 |
| 18 | 6.54, dt (15.5, 7.1)^{c} | 142.9, CH | 19, 17 | 16, 17, 19, 20 |
| 19 | 2.09, q (6.8, | 31.1, CH₂ | 18, 20 | 17, 18, 20, 21 |
| 20 | 1.37, m^{c} | 27.4, CH₂ | 19, 21 | 18, 19, 22 |
| 21 | 1.25, m^{d} | 30.8, CH₂ | 20 | 22 |
| 22 | 1.27, m^{c} | 21.9, CH₂ | 23 | 20, 23 |
| 23 | 0.86, t (7.0) | 13.9, CH₃ | 22 | 21, 22 |
| NH(1) | 8.09, d (8.3) | | 10 | 10, 16 |
| NH(2) | 8.18, d (8.3) | | 4 | 4, 9 |
| NH2 (3) | 9.17, br, s | | | |

^{a}600 MHz for ¹H NMR, ¹³C NMR, COSY, HSQC, HMBC;
^{b}numbers of attached protons were determined by analysis of 2D spectra;
cdetermined through COSY;
^{d}determined through HSQC.

d) Barnesin-L-Phenylalanine (10)

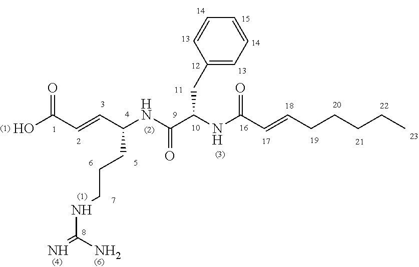 [SMILES: CCCCC/C=C/C(=O)N[C@@H](Cc1ccccc1)C(=O)N[C@@H](/C=C/C(=O)O)CCCNC(=N)N]

TABLE 1E
NMR data of Barnesin-L-Phenylalanine (10) (DMSO-d₆, at 300K)^{a}.

| position | δ_{H}, mult. (J in Hz) | δ_{C}, type^{b} | COSY | HMBC |
|---|---|---|---|---|
| 1 | | 170.6, qC | | |
| 2 | 5.72, d (15.4) | 126.2, CH | 3 | 1, 4 |
| 3 | 6.49, m | 142.3, CH | 2, 4 | 1, 2, 4 |
| 4 | 4.36, m | 48.8, CH | 3, 5, NH(2) | 5, 6, 9 |
| 5a | 1.39, m | 30.7, CH₂ | 4, 5b, 6 | 4, 6, 7 |
| 5b | 1.67, m | | 4, 5a | 4, 6, 7 |
| 6 | 1.46, m | 24.9, CH₂ | 5a, 5b, 7 | 4, 5, 7 |
| 7 | 3.07, m | 40.1^{c}, CH₂ | 6 | 5, 6, 8 |
| 8 | | 157.3, qC | | |
| 9 | | 170.9, qC | | |
| 10 | 4.56, m | 54.3, CH | 11a, 11b, NH(1) | 9, 11, 12, 16 |
| 11a | 2.98, m | 38.2, CH₂ | 10, 11b | 9, 10, 12, 13, 14 |
| 11b | 2.71, m | | 10, 11a | 9, 10, 12, 13, 14 |
| 12 | | 138.0, qC | | |
| 13 | 7.23, m | 129.1, CH | | 11, 13, 14, 15 |
| 14 | 7.23, m | 128.0, CH | 15 | 13, 14, 15 |
| 15 | 7.16, m | 129.7, qC | 14 | 13, 14 |
| 16 | | 164.7, qC | | |
| 17 | 5.89, d (15.5) | 124.3, CH | 18 | 16, 19 |
| 18 | 6.49, m | 142.8, CH | 17, 19 | 16, 17, 19 |
| 19 | 2.06, q (7.3, 14.2) | 31.2, CH₂ | 18, 20 | 17, 18, 20, 21 |
| 20 | 1.35, m | 27.5, CH₂ | 19 | 18, 20, 21 |
| 21 | 1.27, m | 30.8, CH₂ | | 20, 22, 23 |
| 22 | 1.23, m | 21.9, CH₂ | 23 | 20, 21, 23 |
| 23 | 0.85, t (7.0) | 13.9, CH₃ | 22 | 21, 22 |
| NH(3) | 9.79, br. S | | 7 | |
| NH(2) | 8.47, d (8.3) | | 4 | 4, 9 |
| NH(1) | 8.13, d (8.5) | | 10 | 10, 11, 16 |

^{a}600 MHz for ¹H NMR, COSY, HSQC and HMBC; 150 MHz for ¹³C NMR;
^{b}numbers of attached protons were determined by analysis of 2D spectra;
^{c}to be seen in HSQC;
^{d} to be seen in COSY.

### 3.4 Preparation of Comparative Compounds R and S

### 3.4.1 Synthesis of protected 2,3-17,18-Tetrahydrobarnesin (N)

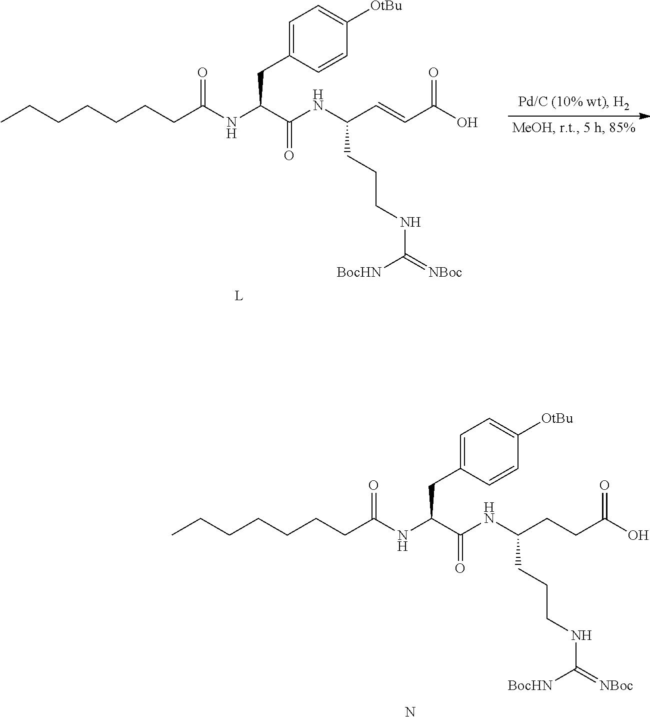

Peptide (L) (16 mg, 21.4 μmol 1.0 eq) was solubilized in methanol and put under argon atmosphere in a dried round-bottom flask. A spatula tip of Pd on carbon (10% weight) was added to the solution. The suspension was put under H₂ atmosphere, and stirred at r.t. for 5 h. An analysis by UPLC-MS confirmed the completion of the reaction. The reaction mixture is filtered over Celite and washed with methanol. The solution was then dried under vacuum to obtain hydrogenated peptide (N) (13.6 mg, 18 μmol 85% yield) as yellowish oil. HRMS (ESI-TOF): calculated for C₃₉H₆₆O₉N₅ [M+H]⁺ 747.4855; found 748.4847.

### 3.4.2 Preparation of Comparative Compound R

2,3,17,18-Tetrahydrobarnesin A (R): According to GPH (see section 3.3.3 above) protected purified 2,3-17,18-tetrahydrobarnesin (N, 12 mg, 16.0 μmol 1.0 eq) was converted to 2,3-17,18-tetrahydrobarnesin (R, 3.8 mg, 7.7 μma 48% yield, colourless oil).

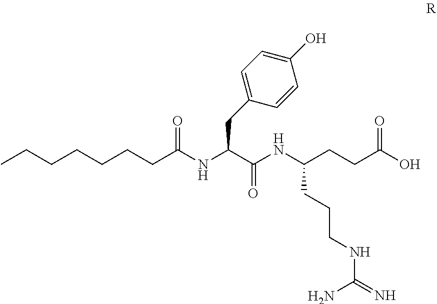 [SMILES: CCCCCCCC(=O)N[C@@H](Cc1ccc(O)cc1)C(=O)N[C@@H](CCCNC(=N)N)CCC(=O)O]

HRMS (ESI-TOF): calculated for C₂₅H₄₂O₅N₅ [M+H]⁺ 492.3180; found 492.3177. IR (ATR) vₘₐₓ: 3288, 2949, 2838, 1644, 1557, 1518, 1449, 1402. [α]_{D}²⁵: +8.3° (c 1.0; MeOH).

### 3.4.3 Preparation of Comparative Compound S

2,3-17,18-Tetrahydrobarnesin A methyl ester (S): Treatment of R with acidic methanol afforded methyl ester (S, 2.9 mg, 5.7 μmol colourless oil).

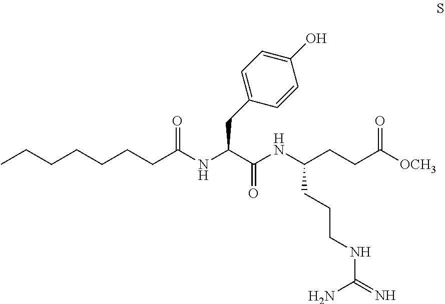 [SMILES: CCCCCCCC(=O)N[C@@H](Cc1ccc(O)cc1)C(=O)N[C@@H](CCCNC(=N)N)CCC(=O)OC]

HRMS (ESI-TOF): calculated for C₂₆H₄₄O₅N₅ [M+H]⁺ 506.3337; found 506.3336. IR (ATR) vₘₐₓ: 3280, 2929, 2856, 1652, 1540, 1517, 1438. [α]_{D}²⁵: +10.22° (c 1.0; MeOH).

### Example 4 Assessment of Protease Inhibition

Protease Assays with azocasein: Protease inhibition assays against the proteases papain, ficin, trypsin, pepsin and thermolysin were performed according to the protocol of Garcia-Carreño (loc. cit.) in a reaction buffer containing 25 mM TRIS-HCl, 0.15 M NaCl, pH 7.2 (or pH 3.5 for aspartic protease). The total assay volume was 47 μL buffer with 2 μL of the protease as well as 1 μL of suitable inhibitor or corresponding inhibitor solvent (100% MeoH or water). Final concentration of proteases was as follows: 2 mg/mL for papain (Sigma-Aldrich, P4762), 600 μg/mL for ficin (Sigma-Aldrich, F6008), 80 μg/mL for trypsin (Thermo Fisher, 23266), 60 mg/mL for pepsin (Sigma-Aldrich P6887)) and 2 μg/mL for thermolysin (Sigma-Aldrich, P1512).

Inhibitors were dissolved in 100% MeOH or water and added to the assay (1 μL); final concentration: 2% MeOH. Samples of control inhibitors were prepared as follows: (1) serine protease (trypsin): PMFS (2 mM) and soybean trypsin inhibitor (240 μM) (Sigma-Aldrich, T1021), (2) cysteine proteases (papain and ficin): iodacetamide (2 mM), (3) asparagine protease (pepsin): pepstatin A (1 μg/mL) (Sigma-Aldrich, P4265), (4) metalloprotease (thermolysin): EDTA (10 mM). In all cases the given concentration inhibited enzyme activity >95%. Negative controls were performed with protease alone (without inhibitor) and MeOH at a final concentration of 2% (reference for activity of 100%). Extract absorbance controls were performed using inhibitor (1 μL in MeOH) without protease. Blanks were performed using 49 μL buffer and 1 μL MeOH.

Samples were incubated with buffer and suitable inhibitor (or without for negative controls) for 1 h at r.t. and the reaction was started afterward by adding 50 μl of 1 azocasein substrate and incubated for 1 h at 37° C. After stopping the reaction with trichloracetic acid (TCA), the separation of the precipitate was accomplished by centrifugation at 6500 g for 5 min. The hydrolyzed substrate supernatant was incubated in an equal volume of 0.5 N NaOH for 5 min and the absorbance was measured at 440 nm. OD values of the protease control without inhibitor were used as reference for a 100% activity.

The log IC₅₀ values were determined using the standard curves analyzing tool with four parameter logistic equation of SigmaPlot12 with technical triplicates. Propagation of error was calculated using the standard error and log IC₅₀ values where the equation of error propagation is defined as Δy=0.43Δx/x.

Cathepsin B inhibition assay. Cathepsin B inhibition assay was determined according to Hiwasa et al. (loc. cit.) with minor changes. Cathepsin B was purchased from Sigma Aldrich (C0150) and stored in 50 mM sodium acetate, 1 mM EDTA, pH 5 (adjusted with acetic acid). Cathepsin B was activated by preincubation at 40° C. for 10 min in assay buffer (0.1 M sodium acetate 1.3 mM EDTA, pH 6.0 adjusted with acetic acid, 2 μM DTT, 2.6 mM cysteine and 0.05% Triton X100). The total assay volume was 47 μL buffer with 2 μL cathepsin B as well as 1 μL of suitable inhibitor or corresponding inhibitor solvent (100% MeOH or water). Final concentration of cathepsin B was 0.1 mg/ml. Samples were incubated with buffer and suitable inhibitor (or without for negative controls) for 20 min at 4° C. and the reaction was started afterwards by adding 1 μL of 10 mM Z-Arg-Arg-AMC (Peptanova, 3123-v) and incubated for 20 min at 40° C. After stopping the reaction with 85 μL buffer containing 100 mM sodium monochlor acetate, 30 mM sodium acetate, 70 mM acetic acid, the hydrolyzed substrate was detected at an excitation wavelength of 380 nm and a fluorescence wavelength of 450 nm, using a fluorophotometer. 1 μL of 1.5 mM leupeptin was used as enzyme inhibition control.

The results of the inhibition assays are summarized in Tables 4A and B below.

TABLE 4A
Evaluation of protease inhibition activities

| | Protease class | | | | | |
|---|---|---|---|---|---|---|
| | (inhibitory activity as IC50 value (μM)) | | | | | |
| | | | | | | Metallo |
| | | | | Serine | Aspartic | (3.4.24) |
| | Cysteine (3.4.22) | | | (3.4.21) | (3.4.23) | Thermo- |
| Compound | Papain | Ficin | Cathepsin B | Trypsin | Pepsin | lysin |
| Barnesin A (1) | 15.96 μM (±5.8) | 3.43 μM (±0.15) | 91.72 nM (±5.8) | n.i. | n.i. | n.i. |
| Barnesin A | 2.89 μM (±0.13) | 3.43 μM (±0.38) | 23.99 nM (±0.13) | n.i. | n.i. | n.i. |
| ethyl ester (3) | | | | | | |
| 17,18- | 4.78 μM (±2.9) | 1.16 μM (±0.07) | 87.56 nM (±2.9) | n.i. | n.i. | n.i. |
| Dihydro- | | | | | | |
| barnesin (4) | | | | | | |
| Comparative | n.i. | n.i. | n.i. | n.i. | n.i. | n.i. |
| Compound (R) | | | | | | |
| Comparative | n.i. | n.i. | n.i. | n.i. | n.i. | n.i. |
| Compound (S) | | | | | | |

TABLE 4B
Reported protease inhibition activities of known compounds

| | Protease class | | | | | |
|---|---|---|---|---|---|---|
| | (inhibitory activity as IC50 value (μM)) | | | | | |
| | | | | | | Metallo |
| | Cysteine (3.4.22) | | | Serine | Aspartic | (3.4.24) |
| | | | Cath- | (3.4.21) | (3.4.23) | Thermo- |
| Compound | Papain | Ficin | epsin B | Trypsin | Pepsin | lysin |
| Leupeptin | 0.86 μM | n.r. | 21.5 nM | 2.2 μM | n.r. | n.r. |
| Cyclo- | 0.0054 μM | n.r. | 0.71 μM | n.r. | n.r. | n.r. |
| propenone | | | | | | |
| 1’S | | | | | | |
| Cyclo- | 22 μM | n.r. | 0.044 μM | n.r. | n.r. | n.r. |
| propenone | | | | | | |
| 1’R | | | | | | |
| Cystatin | 0.029 μM | n.r. | n.r. | 3.5 μM | n.r. | n.r. |
| Miraziri- | n.r. | n.r. | 2.05 μM | 60 μM | n.r. | n.r. |
| dine A | | | | | | |
| Tokar- | n.r. | n.r. | 62.4 nM | n.r. | n.r. | n.r. |
| amide A | | | | | | |
| YM 51084 | 2.2 μM | n.r. | 12.0 nM | n.r. | n.r. | n.r. |

a) n.r. = not reported in literature;
n.i. = no inhibition

Activity Tests Against Cathepsin B, Cathepsin L and Rhodesain.

Rhodesain is a central cysteine protease from Trypanosoma brucei rhodesiense and a potential drug target against human African trypanosomiasis (sleeping sickness).

The determination of the activity of the inhibitors against hCatL and RD was performed in fluorescence-based assays in accordance with the assays described in Giroud et al., ChemMedChem 12 (2017), 257-270 and Schirmeister, Bioorganic & Medicinal Chemistry Letters 27 (2017) 45-50. The biological activities against hCatL were determined using Cbz-Phe-Arg-AMC as substrate, which releases AMC (7-amino-4-methylcoumarin) after amide bond cleavage by the enzyme. The proteolytic activity of the enzyme can be monitored spectrophotometrically by the increase of fluorescence intensity by release of AMC (emission at 460 nm) upon hydrolysis. An initial screen at an inhibitor concentration of 20 mM was performed to identify ligands with an inhibition of hCatL and RD higher than 80%.

TABLE 5
Evaluation of protease inhibition activities

| | Cathepsin B | Cathepsin L | Rhodesain |
|---|---|---|---|
| Compound | (%) | (%) | (%) |
| 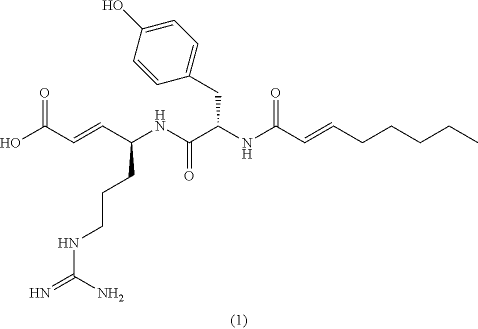 [SMILES: CCCCC/C=C/C(=O)N[C@@H](Cc1ccc(O)cc1)C(=O)N[C@H](/C=C/C(=O)O)CCCNC(=N)N] | 50 | 95 | 99 |
| 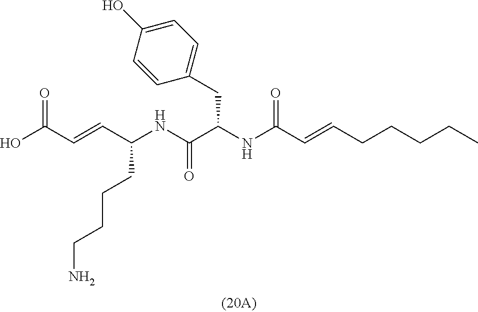 [SMILES: CCCCC/C=C/C(=O)N[C@@H](Cc1ccc(O)cc1)C(=O)N[C@@H](/C=C/C(=O)O)CCCCN] | 73 | 96 | 9 |
| 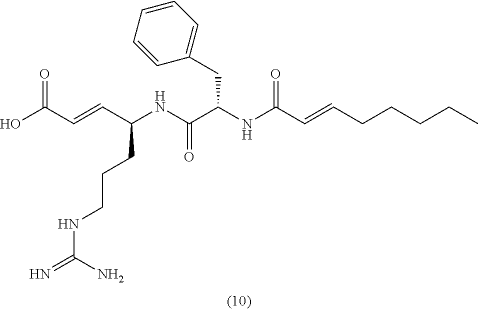 [SMILES: CCCCC/C=C/C(=O)N[C@@H](Cc1ccccc1)C(=O)N[C@H](/C=C/C(=O)O)CCCNC(=N)N] | 41 | 90 | 99 |
| 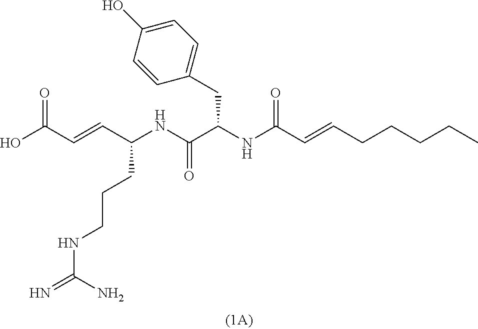 [SMILES: CCCCC/C=C/C(=O)N[C@@H](Cc1ccc(O)cc1)C(=O)N[C@@H](/C=C/C(=O)O)CCCNC(=N)N] | 15 | 20 | 60 |
| 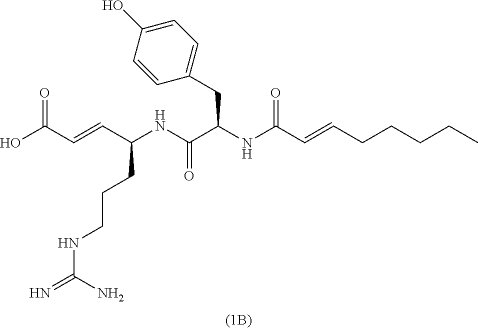 [SMILES: CCCCC/C=C/C(=O)N[C@H](Cc1ccc(O)cc1)C(=O)N[C@H](/C=C/C(=O)O)CCCNC(=N)N] | 5 | 7 | ni |
| 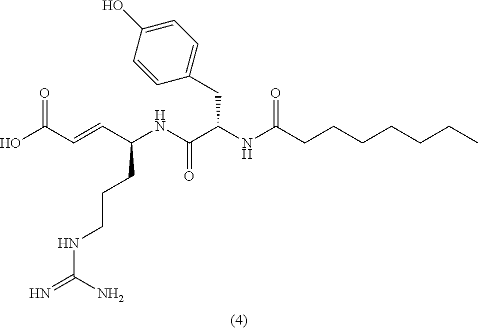 [SMILES: CCCCCCCC(=O)N[C@@H](Cc1ccc(O)cc1)C(=O)N[C@H](/C=C/C(=O)O)CCCNC(=N)N] | 30 | 80 | 99 |

### Example 5 Determination of Metabolic Stability

Glutathione (GSH) Assay:

Glutathione (GSH), a thiol-containing tripeptide (γ-glutamyl-cysteinyl-glycine), is a key antioxidant in many species. It has been highly implicated in the detoxification/elimination of antibiotics and xenobiotics (naturally occurring harmful compounds such as free radicals, hydroperoxides etc.) and in the maintenance of the oxidation state of protein sulfhydryl groups. In addition, GSH plays a pivotal role in the pathogenesis of numerous human diseases including cancer and cardio-vascular diseases. Glutathione is present in cells in both reduced (GSH) and oxidized (GSSG) forms—GSH being, the predominant species under normal physiological conditions inside cells. Furthermore, electrophiles that cause the depletion of the cellular GSH pool can cause cytotoxicity.

Procedure: A standard GSH assay includes a phosphate buffer (720 μL), a GSH solution (40 μL, 100 mM) and the test solution of the test compound (20 μM in 10% DMSO in phosphate buffer (100 mM, pH 7.4)). As negative control (NC) serves the test item (20 μM in 10% DMSO in phosphate buffer (100 mM, pH 7.4) in phosphate buffer (760 μL).

Samples are prepared as follows: phosphate buffer (720 μL) and GSH solution (40 μL, 100 mM) were preincubated at 37° C., and 40 μL of the test item solution (or internal standard) added. Then 100 aliquots of the assay mixture were mixed with 100 μL ACN were at the following time points: t=0, 30 and 90 min; in case of the test item, t=0 and 90 min in case of PC or NC. The samples were mixed for 2 min at 150 rpm, centrifuged at 16.1 (krcf). 5 μL of each samples were injected to a UPLC/HRMS system. The peak areas of the extracted ion chromatograms were determined using the standard software.

The results of the assay are shown in Table 6 below. As will be recognized, the compounds of the invention, i.e. barnesin and derivatives thereof, are remarkably unreactive towards general thiol nucleophiles such as GSH. In other words, the compounds of the invention have a high stability towards soft nucleophiles. More specifically, an intracellular detoxification mechanism, unwanted side reactions and unregulated depletion of the GSH pool by simple, unselective 1,4-addition of GSH to the vinylogous double bond of the claimed compounds can be excluded.

Thus, the experimental data demonstrate that the compounds of the invention have an advantageous pharmacokinetic property, namely high stability towards soft nucleophiles.

TABLE 6
Metabolic stability in GSH Assay

| | | | Time | | Area | Area rel. |
|---|---|---|---|---|---|---|
| Test item | Sample | Type | [min] | Area | rel. | [%] |
| 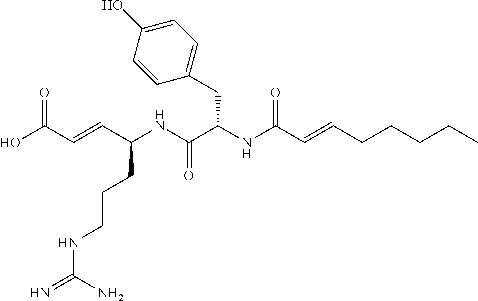 [SMILES: CCCCC/C=C/C(=O)N[C@@H](Cc1ccc(O)cc1)C(=O)N[C@H](/C=C/C(=O)O)CCCNC(=N)N] | FM049_1 FM049_7 FM049_10 FM049_4 FM049_13 | Test item NC | 0 30 90 0 90 | 367407693 342890085 345965508 363205474 333706916 | 1.000 0.933 0.942 1.000 0.919 | 100.0 93.3 94.2 100.0 91.9 |
| 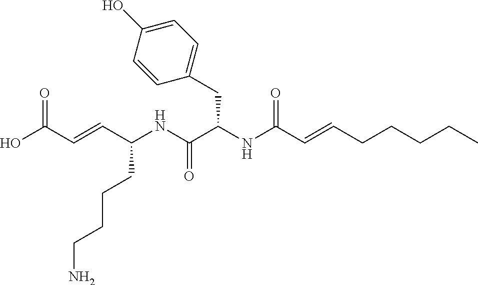 [SMILES: CCCCC/C=C/C(=O)N[C@@H](Cc1ccc(O)cc1)C(=O)N[C@@H](/C=C/C(=O)O)CCCCN] | FM049_2 FM049_8 FM049_11 FM049_5 FM049_14 | Test item NC | 0 30 90 0 90 | 519869606 542406698 509683755 510530074 530330916 | 1.000 1.043 0.980 1.000 1.039 | 100.0 104.3 98.0 100.0 103.9 |
| 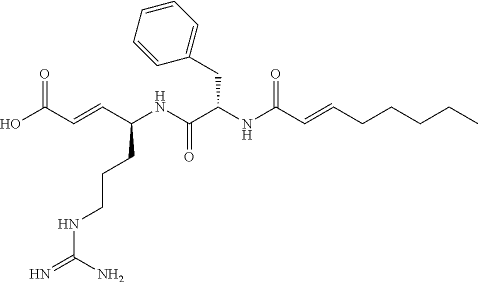 [SMILES: CCCCC/C=C/C(=O)N[C@@H](Cc1ccccc1)C(=O)N[C@H](/C=C/C(=O)O)CCCNC(=N)N] | FM049_3 FM049_9 FM049_12 FM049_6 FM049_15 | Test item NC | 0 30 90 0 90 | 680377355 698665622 722442552 705274108 717968682 | 1.000 1.027 1.062 1.000 1.018 | 100.0 102.7 106.2 100.0 101.8 |
| 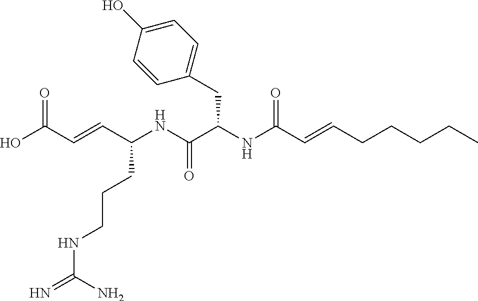 [SMILES: CCCCC/C=C/C(=O)N[C@@H](Cc1ccc(O)cc1)C(=O)N[C@@H](/C=C/C(=O)O)CCCNC(=N)N] | FM062_29 FM062_35 | Test item | 0 90 | 202656073 201316491 | 1.000 0.993 | 100.0 99.3 |
| 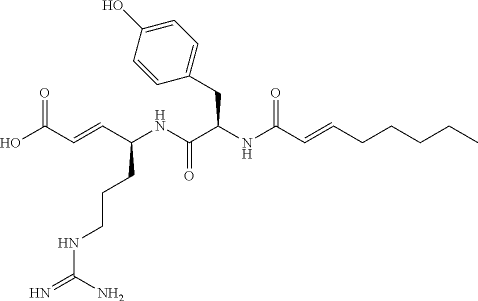 [SMILES: CCCCC/C=C/C(=O)N[C@H](Cc1ccc(O)cc1)C(=O)N[C@H](/C=C/C(=O)O)CCCNC(=N)N] | FM062_30 FM062_36 FM062_40 FM062_42 | Test item NC | 0 90 0 90 | 115525226 113959407 117560798 108084764 | 1.000 0.986 1.000 0.919 | 100.0 98.6 100.0 91.9 |
| 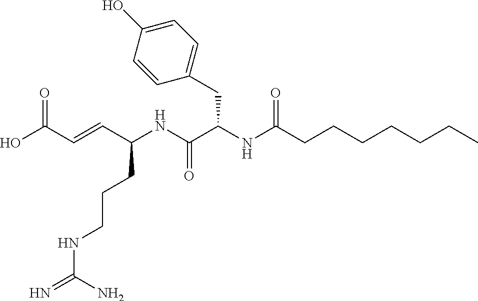 [SMILES: CCCCCCCC(=O)N[C@@H](Cc1ccc(O)cc1)C(=O)N[C@H](/C=C/C(=O)O)CCCNC(=N)N] | FM062_31 FM062_37 | Test item | 0 90 | 489372587 507468070 | 1.000 1.037 | 100.0 103.7 |
| 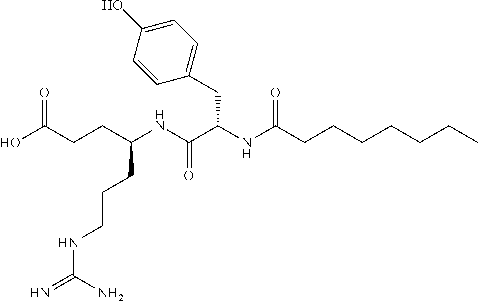 [SMILES: CCCCCCCC(=O)N[C@@H](Cc1ccc(O)cc1)C(=O)N[C@@H](CCCNC(=N)N)CCC(=O)O] | FM062_32 FM062_38 | Test item | 0 90 | 501743817 516317782 | 1.000 1.029 | 100.0 102.9 |
| 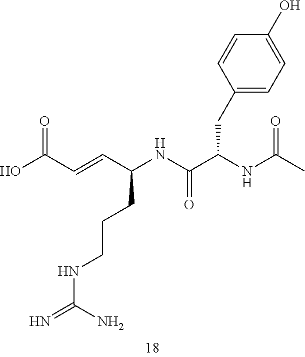 [SMILES: CC(=O)N[C@@H](Cc1ccc(O)cc1)C(=O)N[C@H](/C=C/C(=O)O)CCCNC(=N)N] | FM062_33 FM062_39 FM062_41 FM062_43 | Test sample NC | 0 90 0 90 | 40293650.6 45445179.6 37458620.2 40800514.3 | 1.000 1.128 1.000 1.089 | 100.0 112.8 100.0 108.9 |
| IR76 | FM062_28 | PC | 0 | 29338493 | 1.000 | 100.0 |
| (internal standard) | FM062_34 | | 90 | 17109.4453 | 0.001 | 0.1 |

Microsome Stability Assay

The liver is the main organ of drug metabolism in the body. Subcellular fractions such as liver microsomes are useful in vitro models of hepatic clearance as they contain many of the drug metabolising enzymes found in the liver. Liver microsomes are subcellular fractions which contain membrane bound drug metabolising enzymes.

The microsomes are incubated with the test compound at 37° C. in the presence of the co-factors, which initiates the reaction. The reaction is terminated by the addition of organic solvents containing internal standard. Following centrifugation, the supernatant is analysed on the LC-MS/MS. The disappearance of test compound is monitored over certain time period.

Here, a microsome stability assay was used to investigate the metabolism of the compounds; using this assay it is possible to measure in vitro the intrinsic clearance or to identify metabolites formed.

For a standard assay the following solutions were used: 360 μL Microsomal solution (1.1 mg/mL in phosphate buffer), 360 μL NADP-regeneration mix (containing NADP (10 mM), MgCl₂ (50 mM), glucose-6-phosphate (50 mM), glucose-6-phosphate dehydrogenase (50 U/ml)) and 40 μL phosphate buffer (100 mM, pH 7.4). Diclofenac (10 μL, 200 μM) was used as positive control (360 μL Microsomal solution, 360 μL NADP-regeneration mix and 40 μL Phosphate buffer) and phosphate buffer (360 μL Microsomal solution, 360 μL NADP-regeneration mix) served as negative control. The test substance was dissolved to 20 μM in 10% DMSO (phosphate buffer (100 mM, pH 7.4)) and used as working solution.

The following procedure was applied: 40 μL of the working solution was added to a pre-incubated solution containing the microsomal solution and the NADP-regeneration mix at 37° C./1500 rpm. Samples were prepared as follows: Proteine precipitation was induced by addition of 100 μL ACN to 100 μL aliquots of the assay mixture at t=0, 30 and 90 min in case of the test item, t=0 and 30 min in case of PC and t=0 and 90 min in case of NC. Samples were mixed at 1500 rpm for 2 min, centrifuged for 2 min at 16.1 krcf. 5 uL of each sample was injected to a UPLC/HRMS system and the peak areas of the extracted ion chromatograms were determined

TABLE 7
Metabolic stability in liver microsomes

| | | | Time | | Area | Area rel. |
|---|---|---|---|---|---|---|
| | Sample | Type | [min] | Area | rel. | [%] |
| 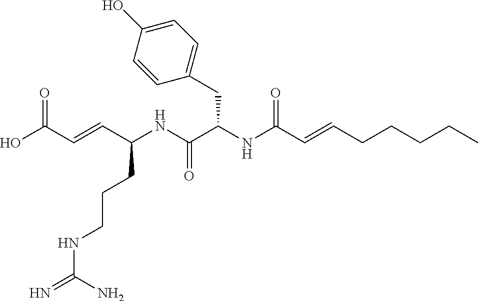 [SMILES: CCCCC/C=C/C(=O)N[C@@H](Cc1ccc(O)cc1)C(=O)N[C@H](/C=C/C(=O)O)CCCNC(=N)N] | FM052_13 FM052_19 FM052_22 FM052_16 FM052_25 | Sub- stance NC | 0 30 90 0 90 | 235792454 208205685 196717888 211564777 195341003 | 1.000 0.883 0.834 1.000 0.923 | 100.0 88.3 83.4 100.0 92.3 |
| 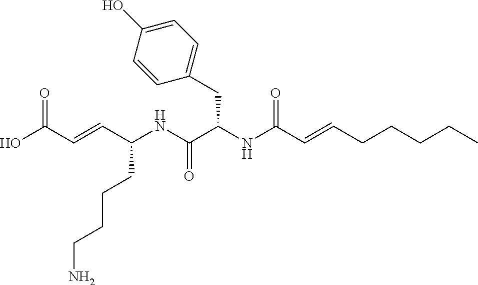 [SMILES: CCCCC/C=C/C(=O)N[C@@H](Cc1ccc(O)cc1)C(=O)N[C@@H](/C=C/C(=O)O)CCCCN] | FM052_14 FM052_20 FM052_23 FM052_17 FM052_26 | Sub- stance NC | 0 30 90 0 90 | 361788562 298891411 288411590 336394843 322058861 | 1.000 0.826 0.797 1.000 0.957 | 100.0 82.6 79.7 100.0 95.7 |
| 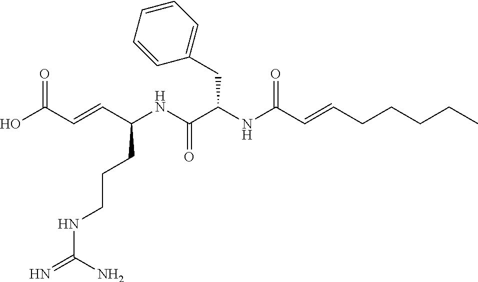 [SMILES: CCCCC/C=C/C(=O)N[C@@H](Cc1ccccc1)C(=O)N[C@H](/C=C/C(=O)O)CCCNC(=N)N] | FM052_15 FM052_21 FM052_24 FM052_18 FM052_27 | Sub- stance NC | 0 30 90 0 90 | 330765136 281767935 262648398 280077767 249809907 | 1.000 0.852 0.794 1.000 0.892 | 100.0 85.2 79.4 100.0 89.2 |
| Diclo | FM052_10 | PC | 0 | 4.18E+08 | 1.000 | 100.0 |
| | FM052_11 | | 10 | 2.05E+08 | 0.490 | 49.0 |
| | FM052_12 | | 35 | 1.60E+07 | 0.038 | 3.8 |
| 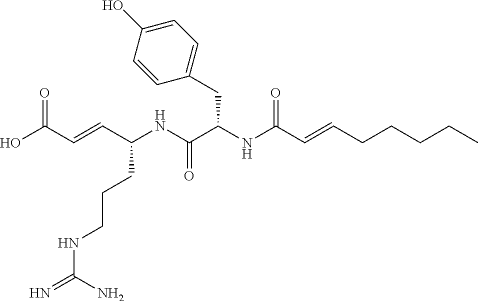 [SMILES: CCCCC/C=C/C(=O)N[C@@H](Cc1ccc(O)cc1)C(=O)N[C@@H](/C=C/C(=O)O)CCCNC(=N)N] | FM062_1 FM062_7 FM062_13 FM062_18 FM062_23 | Sub- stance NC | 0 30 90 0 90 | 179467850 142403215 121177629 143469802 147452392 | 1.000 0.793 0.675 1.000 1.028 | 100.0 79.3 67.5 100.0 102.8 |
| 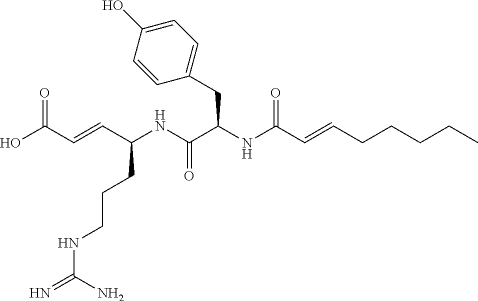 [SMILES: CCCCC/C=C/C(=O)N[C@H](Cc1ccc(O)cc1)C(=O)N[C@H](/C=C/C(=O)O)CCCNC(=N)N] | FM062_2 FM062_8 FM062_14 FM062_19 FM062_24 | Sub- stance NC | 0 30 90 0 90 | 95241639.3 85162953 82923793.2 84799508 89736887.8 | 1.000 0.894 0.871 1.000 1.058 | 100.0 89.4 87.1 100.0 105.8 |
| 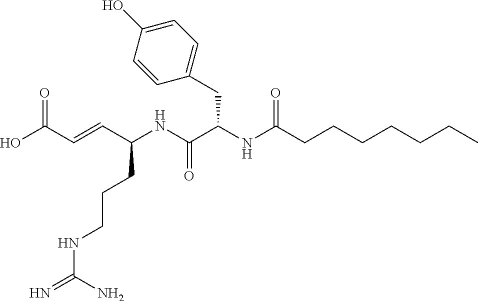 [SMILES: CCCCCCCC(=O)N[C@@H](Cc1ccc(O)cc1)C(=O)N[C@H](/C=C/C(=O)O)CCCNC(=N)N] | FM062_3 FM062_9 FM062_15 FM062_20 FM062_25 | Sub- stance NC | 0 30 90 0 90 | 328347157 304396247 302016018 332191329 346639421 | 1.000 0.927 0.920 1.000 1.043 | 100.0 92.7 92.0 100.0 104.3 |
| 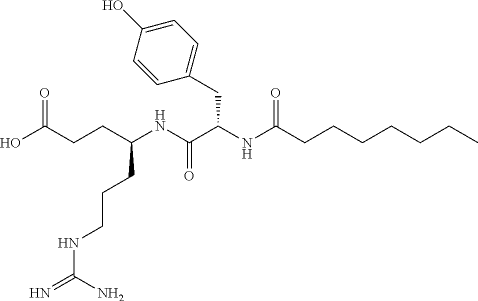 [SMILES: CCCCCCCC(=O)N[C@@H](Cc1ccc(O)cc1)C(=O)N[C@@H](CCCNC(=N)N)CCC(=O)O] | FM062_4 FM062_10 FM062_16 FM062_21 FM062_26 | Sub- stance NC | 0 30 90 0 90 | 396622297 379045795 389518824 363662768 361941845 | 1.000 0.956 0.982 1.000 0.995 | 100.0 95.6 98.2 100.0 99.5 |
| 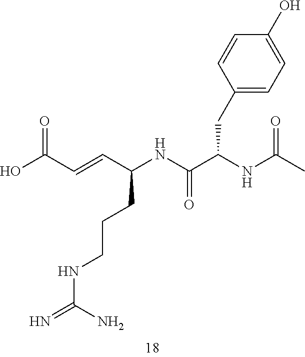 [SMILES: CC(=O)N[C@@H](Cc1ccc(O)cc1)C(=O)N[C@H](/C=C/C(=O)O)CCCNC(=N)N] | FM062_5 FM062_11 FM062_17 FM062_22 FM062_27 | Sub- stance NC | 0 30 90 0 90 | 38412530.4 33877398.1 38180235.5 25518415.7 28546900.9 | 1.000 0.882 0.994 1.000 1.119 | 100.0 88.2 99.4 100.0 111.9 |

As can be taken from Table 7, all compounds have a good metabolic stability compared to the positive control. In essence, the compounds of the invention are able to reach the cellular target and are not metabolist instantaneously. Accordingly, this data shows that the compounds of the invention have good pharmacokinetic properties which makes them suitable as active agents in various applications, including oral drugs.

The above results confirm that the compounds according to the invention act as selective cysteine protease inhibitors in the low molecular range.

The features of the present invention disclosed in the specification, the claims and/or the drawings may both separately and in any combination thereof be material for realizing the invention.

1. A compound of the general formula (IA): 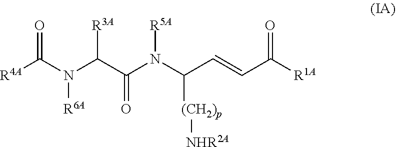
or a pharmacologically acceptable salt thereof, wherein
R^{1A} represents a hydrogen atom, —OR¹¹, —NR¹¹R¹²; or a (C₁-C₆)alkyl, (C₂-C₆)alkenyl, (C₂-C₆) alkinyl, or (C₃-C₆) cycloalkyl group, all of which groups may optionally be substituted;
R¹¹ and R¹² each, independently of one another, represents a hydrogen atom, or a (C₁-C₆)alkyl, (C₂-C₆)alkenyl, (C₂-C₆) alkinyl or (C₁-C₆)heteroalkyl group, all of which groups may optionally be substituted, or R¹¹ and R¹² together with the nitrogen atom to which they are attached form a 5- to 8-membered heterocyclic or heteroaromatic ring that can be substituted with from 0 to 3 substituents which substituents are each independently selected from halogen atom, —OH, —NH₂, —NHC₁₋₆ alkyl, and —N(C₁₋₆ alkyl)₂;
R^{2A} is a hydrogen atom, a group of formula —C(═NH)NH₂, or a group of formula —C(═O)R²¹;
R²¹ represents a (C₁-C₆)alkyl, (C₂-C₆)alkenyl, (C₂-C₆) alkinyl or (C₁-C₆)heteroalkyl group; all of which groups may optionally be substituted;
R^{3A} is an amino acid side chain; a hydrogen atom, a halogen atom, OH; or an alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl group; all of which groups may optionally be substituted;
R^{4A} is a hydrocarbon group containing 1 to 12 carbon atoms or a heteroaryl group containing from 5 to 10 ring atoms, and, optionally, 1 to 3 H atoms in the hydrocarbon and the heteroaryl group may, independently of each other, be replaced by a halogen atom, OH, NH₂, —NHCH₃, —N(CH₃)₂, —NH(OC₁₋₃ alkyl), unsubstituted C₁-C₃alkyl, (C₁-C₃)haloalkyl, (C₁-C₃)hydroxyalkyl, or (C₁-C₃)alkoxy group;
R^{5A} and R^{6A} each, independently of one another, represents a hydrogen atom or a methyl group; and
p is an integer of from 1 to 6.

2. The compound according to claim 1, or a pharmacologically acceptable salt thereof, wherein R^{1A} represents —NH₂, —NHCH₃, —N(CH₃)₂, —NH(OC₁₋₃ alkyl), —NCH₃(OC₁₋₃ alkyl), —NH(C₁₋₃ alkyl)CN; 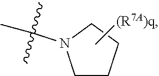
wherein each R^{7A} is independently selected from halogen atom, —OH, —NH₂, and —NHC₁₋₃ alkyl, and q is an integer of from 0 to 3; a C₁₋₃ alkoxy group; or —OH.

3. The compound according to claim 1, or a pharmacologically acceptable salt thereof, wherein R^{2A} is a hydrogen atom, a group of formula —C(═NH)NH₂, a group of formula —C(═O)CH₃, or a group of formula —C(═O)CH₂CH₂CH₃.

4. The compound according to claim 1, or a pharmacologically acceptable salt thereof, wherein R^{3A} is an optionally substituted amino acid side chain of a proteinogenic amino acid; or a group of formula (II): 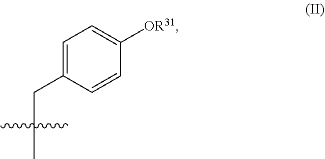
wherein R³¹ represents a (C₁-C₆)alkyl, (C₂-C₆)alkenyl, or (C₂-C₆) alkinyl group, all of which groups may optionally be substituted.

5. The compound according to claim 1, or a pharmacologically acceptable salt thereof, wherein R^{3A} represents the amino acid side chain of tyrosine, a group of formula (II); or an amino acid side chain of phenylalanine, leucine or isoleucine, wherein 1 to 3 H atoms in the respective side chain group may, independently of each other, be replaced by a halogen atom, OH, NH₂, —NHCH₃, —N(CH₃)₂, —NH(OC₁₋₃ alkyl), unsubstituted C₁-C₃alkyl, (C₁-C₃)haloalkyl, (C₁-C₃)hydroxyalkyl, or (C₁-C₃)alkoxy group.

6. The compound according to claim 1, or a pharmacologically acceptable salt thereof, wherein R^{4A} is a C₁₋₇ alkyl, C₂₋₇ alkenyl, (C₂-C₇) alkynyl, cyclohexyl, phenyl, benzyl or pyridyl group; wherein 1 to 3 H atoms in said groups may, independently of each other, be replaced by a halogen atom, OH, NH₂, —NHCH₃, —N(CH₃)₂, —NH(OC₁₋₃ alkyl), unsubstituted C₁-C₃alkyl, (C₁-C₃)haloalkyl, (C₁-C₃)hydroxyalkyl, or (C₁-C₃)alkoxy group.

7. The compound according to claim 1, or a pharmacologically acceptable salt thereof, wherein p is 3 or 4.

8. The compound according to claim 1, wherein R^{5A} and R^{6A} represent a hydrogen atom.

9. The compound according to claim 1, wherein the compound is: 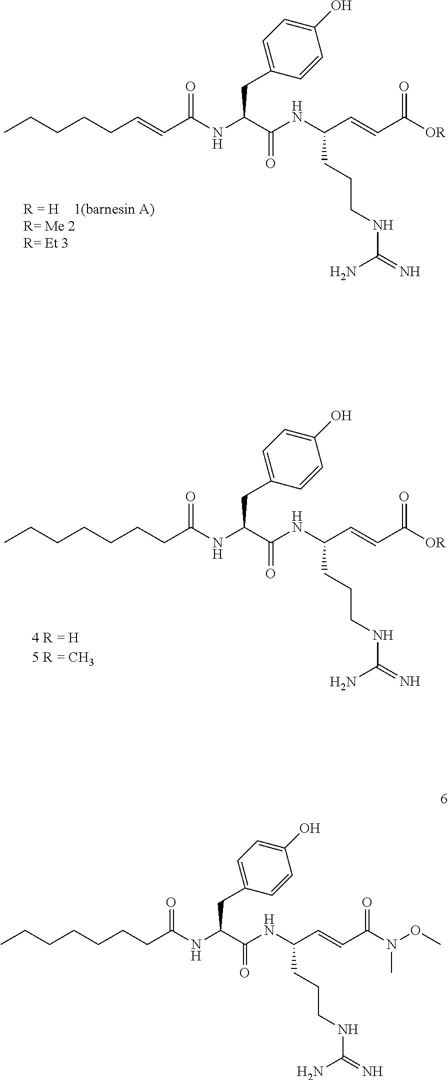 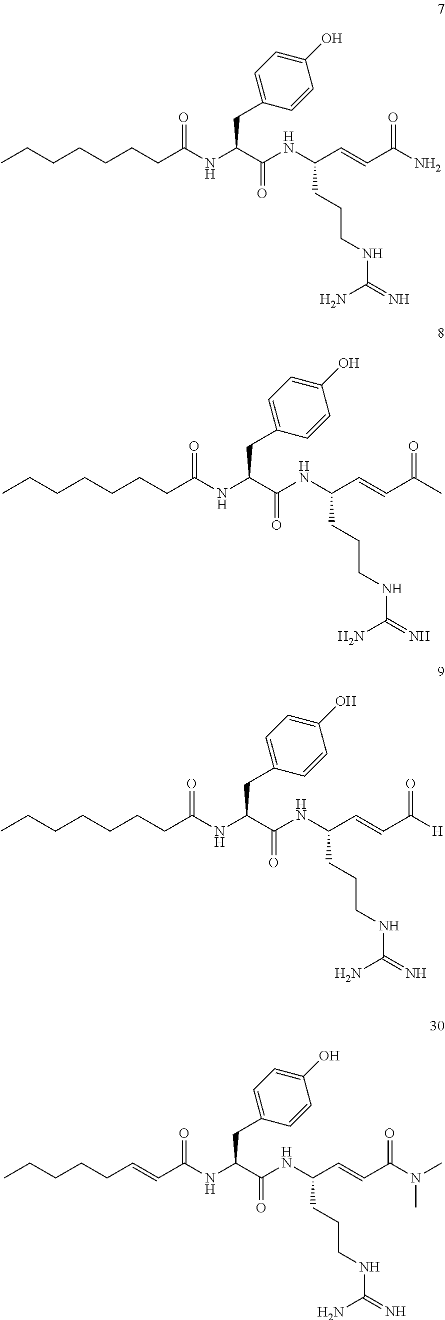 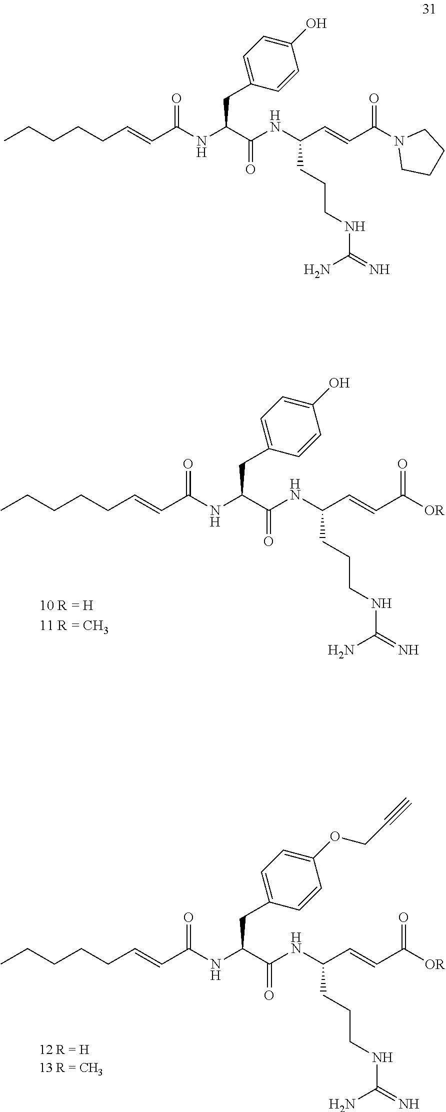 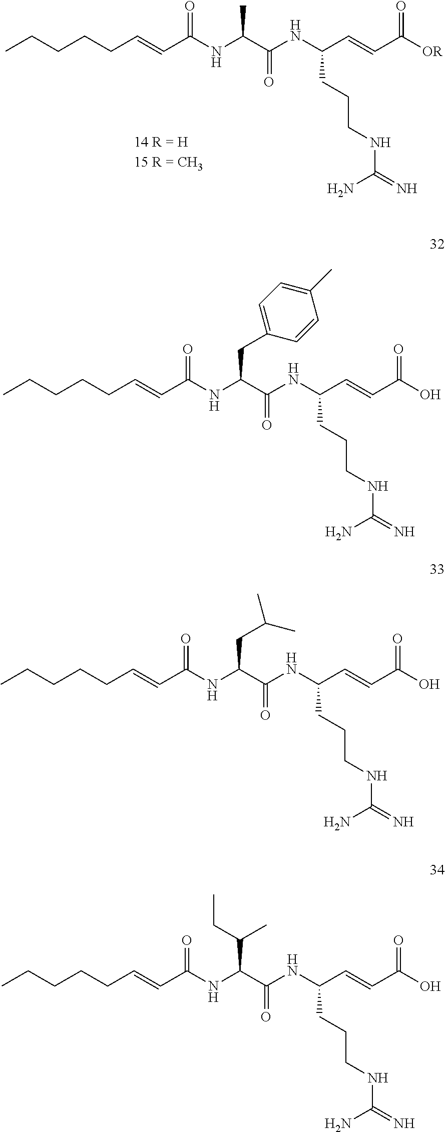 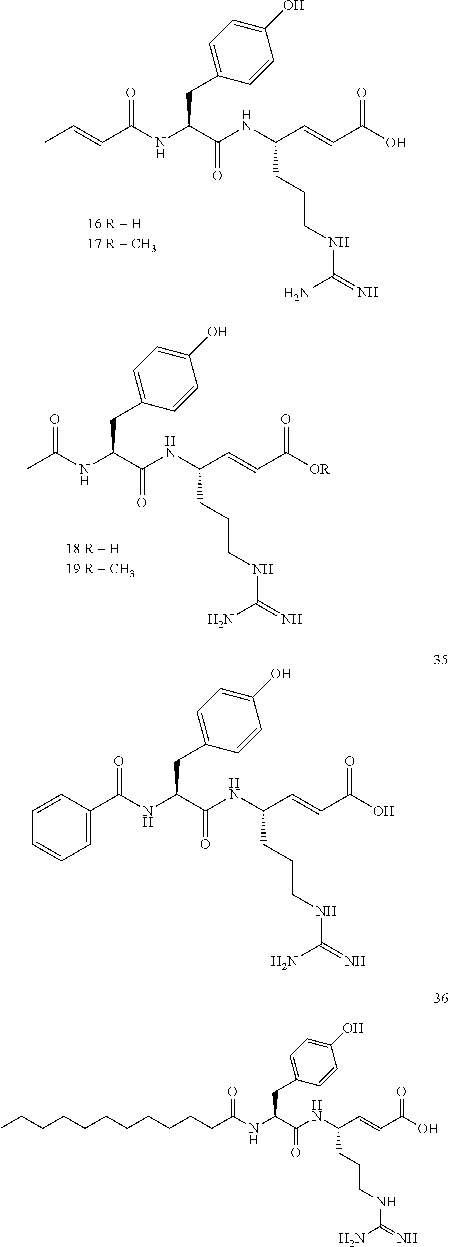 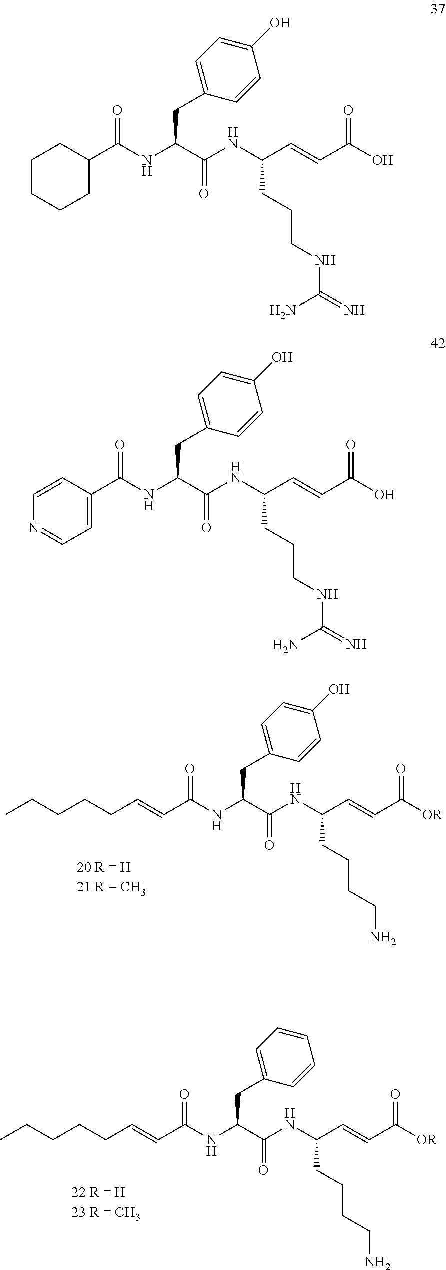 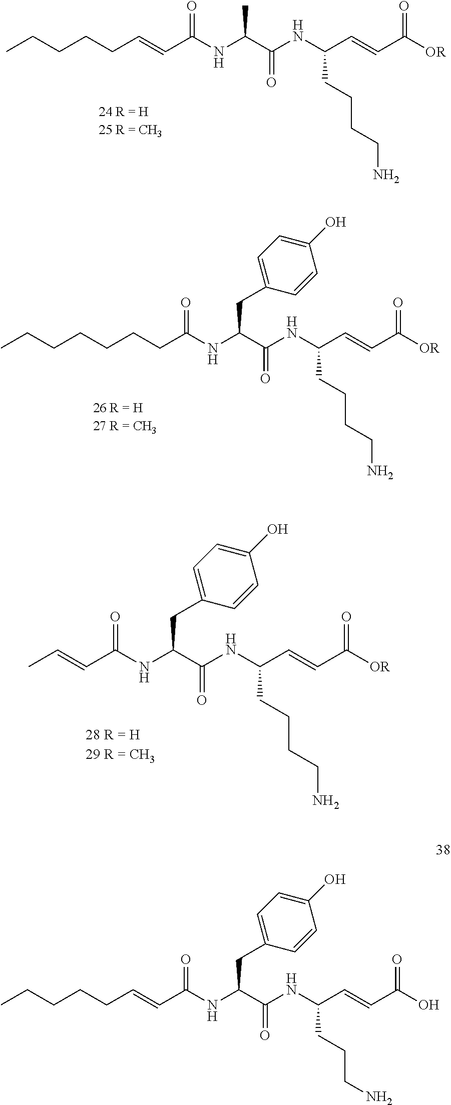 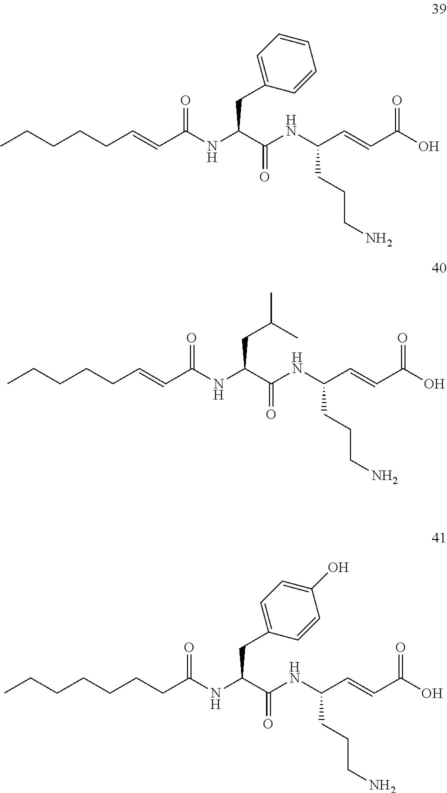 [SMILES: CCCCC/C=C/C(=O)N[C@@H](Cc1ccccc1)C(=O)N[C@H](/C=C/C(=O)O)CCCN ; CCCCC/C=C/C(=O)N[C@@H](CC(C)C)C(=O)N[C@H](/C=C/C(=O)O)CCCN ; CCCCCCCC(=O)N[C@@H](Cc1ccc(O)cc1)C(=O)N[C@H](/C=C/C(=O)O)CCCN]
or a pharmacologically acceptable salt thereof.

10. A pharmaceutical composition comprising at least one compound according to claim 1 and, optionally, one or more carrier substance(s), excipient(s) and/or adjuvant(s).

11. A combination preparation containing at least one compound according to claim 1 and at least one further active pharmaceutical ingredient.

12. The compound according to claim 1 for use as a medicament.

13. The compound according to claim 1 claim 10 claim 11 for use in the prevention and/or treatment of a condition or disorder associated with a pathophysiological level of a proteasome or a cysteine protease.

14. The compound for use according to claim 13, wherein the condition or disorder associated with a pathophysiological level of a proteasome or a cysteine protease is a neurodegenerative disorder, a parasitic infection, an invasive cancer, or a metastatic cancer.

15. The compound according to claim 1, or the pharmaceutical salt thereof, for use as an inhibitor of a proteasome or a cysteine protease.

16. A synthetic nucleic acid comprising a sequence encoding a nonribosomal peptide-synthetase (NRPS)-polyketide synthase (PKS) gene cluster capable of synthesizing compound (1) of claim 9, wherein the sequence has a sequence identity to the full-length sequence of SEQ ID NO. 1 from at least 90%, 95%, 96%, 97%, 98%, 98.5%, 99%, or 99.5% to 100%.

17. A method for the preparation of compound (1) of claim 9, the method comprising the steps of:
(a) fermenting Sulfurospirillum barnesii (DSM 10660); and
(b) separating and retaining the compound according to general formula (I) from the culture broth.

18. A method of treating a subject who is suffering from or susceptible to a condition or disorder associated with a pathophysiological level of a proteasome or a cysteine protease, comprising administering to a patient in need thereof an effective amount of a compound of claim 1.

19. A method of treating a subject who is suffering from or susceptible to a neurodegenerative disorder, a parasitic infection, an invasive cancer, or a metastatic cancer, comprising administering to a patient in need thereof an effective amount of a compound of claim 1.


SEQUENCE LISTING (1 sequence)

SEQ ID NO: 1 - DNA, 16035 bp, Artificial Sequence
  Barnesin A expression cassette
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatataccatggatgcttaatatatacgcactttcatcaccacaagaggctatatatattgactcccttttgtatagtgaaaccacaaaatatactatgggtggctttgctctcttaagtggtcggattgatgttgagatatttaaaaaagcccatgccattacactgacccttcacgatgtatttctttccaaaatagagacaaggcatgggaaaattgtacaaagctctccacatgaaccatatggggtatattttaaggatttttccacttccgttgattcatacagggcaggtgttgagttcattcttgaagatttttctcatcccatacccatcaatgcatacccccttgcagcagatatgctactcaaagtgaatgaagagctttatatttggtacaccaaactgcatcatattatgaacgacgcatttggtcatgctctttttgcagaaactttatctgaaatttatacagctttgttttataagcaagagttgccagtaaaagaaaggcacagatacgttgattttgttgaagatgacaacgcatatctccattccgaaacctttttaaaagatgcagcattttggatggagaagtttaagactctacctgaacctatttctttttcaggttcaaaagatggactttgtgagcaagaagcattaaaaacggaaagaatgacattaacacttagcagattgtgttacaacgatatgctcaggatttgtagcgaaaatagttttacctcctttcattttcttcttgccgttctttatacgtatttgtatagaacaaaaaaccgcaacgatattgttattggcatgcccattcttaatcgcaataataaaaaatttagaaacacttcgggtatgtttatgggtatgataccccttcgtattaaaatggaagagggtatgagttttatggatctagcgcttggtataagccgtgaacttagggagtgttatcggcatcagcgttaccctcttggtcagataataaaggattgccgtagcaaagaggggttttatggtaacatttttgatataacatttgtgtaccgtaaactttcctatgataagaagtttggaaaaactcttatgcgcatgcaaatacttgatacaaaagcgagggaagaatccttttccgttgaagtagatgagtttgacgatggcgatgtgaatcttttttttaattacaatccgtatgttattcctccagaagaggcagaacagatggttagagggtttgaaaccctttttttagatgttgcaatgatggaagataagcctcttaaagagcttagaattatgcctgaaagagtgaggtgtggggtttttggagaaaaaattaactttcctaaaaagcgatttgaagaaatctttgcacagacatgtatgcagtatggcgctaaagaagccgtaaaatacaaagacactactcttacatacgttgaattagatggcttctcttcaggcatcgccaaaaaactaagagacgattttgggctacaaaaaggtgacagggttgtttgtgttcttgagcgtactcacattgtgccagtggtgatacttgccctttttaaaatggggtgtgtgtatgttccagccgatacgtcccttcctaaagatcgtatcaattacatggtaaatgattgtgcagcaaagctaattatcagttcagctcctcttgaagcagaggtttgcgtctatatacccgatactctagtttcaggcttttttgcacccatagagagtttaaatgctgaagatgatgcttatatcatatacacttctggaacaacaggtcgtcctaaaggggttgttgtaccgcatcaaggaatagtcaatacggtgttgtcacaagcaagtcaatgggagggtggtaagcatgccaatgttctccaatttgcttctttaggatttgatgcctccttgtcagagattgggatggcgctccttagtggcgcatccttaaagatcgtgccaaagcagacaattctaagtcctgctgagtttatagattttatgaacaaagaaaaggttacagtagcaacattgcctccttcttatctgagttcattagggttccctgaatttttttatcttaaaagcttaataaccgcaggtgaatcacccatagaaaaagatgttcttttttacaaaaacctctgtcgtgtcatcaacgcatacggacccacagaaatatcagtgtgcgcaagttggtacgaaatccccaaagactattgtgggggcgctgttcccataggaaaagcaattgataatgcacatatttatattttggatgataacctcaacccccttccgcttggttctgtggggcagatatgtgtaggaggagcaggtgttactaggggataccttaataatccagaactaacgcagcaaacatttcaaaaagacctcttttttgaggggcagaaaatgtatctaacgggggatatggggaaaattaatgctgatggtaaccttgtgtttttaggaagaaaagatgcacagattaagcttaggggttatcgcattgagccagaagaggtagcacgtgtgatggaggggctttcttttatagatgccacatccattgatgttcacgacgaaggtgaaagcaaaagccttgtagcgtattacactgcaaaaaatgtagttgatgttgcgtatattaaaaaggcacttttggaaaaattacctccttatatgattccctcattttttatacatgtaaaagaatttcctcttaatgttagtgggaaaatagacaaaaaagcactcaaaggggcatttaaaaaaataccttcttctacaaaagagctccaacttccgctaagtgaaacagagcagaatgtttctcgtgtgtggtcagaaattcttgagatagatcactttgaaacggatacaaacttttttgatttaggtgggcattctttgcatgcgattaaggtcatgagcaaactgttcactctctttggtgtgcgcattgggttaagggagttttttgcagacccaacgcttaagggtattgcttctattctggacaaaaggagcggcaataaagagaaaaaaatcgcaaatcttgcattgccacataccagagcgttaagtccatcggaaagacgtatttgggtgttttgcgcaatgcaaggtagctcttctgcttataatatgcctcttctcatggacattgaaggagaggtggatgtaggtgcgctaaacagtgctcttaagcttattctctcacggcatgatattttaagaacctattatgaagaagaaagggctgtgtgccacatcaaaagtgagtgcatgtttgaagttcaagaaatcgtcaccaaaaacctcactgaggcactcttgcatgagattgatgaacctttcaatttatacgatgcgccccttttcaggttgaagatattgactgatggaaccaaaaaaacattgagcttcgtgattcatcacatcatttcagacggttggtcgcttcaagtgttgatgaacgaattgcttcaagcctacgaagcggtgaaaaaaagagaagtgcccgagctttcaccaacaaatgtttcttacgcctcttatgccacatggttagagagtgaagagtatcaagagcttgcgaggtcggatagagcttattggcttgagcgctttaagcatatgcctcctgagttggaattgccgatagatttttcacgaggttcgatgcttagtttcaaaggtgcatccattcacagagattttgaattgcgttgggatgctatttcgcataaggcagcgacccttgatacaacaccttttacactctttcttgcagcactttatggggtgttgcatacctattcaagctcaacagatattgtaattggttctcctgtctcagggcggattcatcctgatattcaaaaaacatgtggcattttcataaacacgctcccacttagggtaacctttagtcctacagaaaactttttaaccctagtagccagaacaaaaacagccatcaccgaagggcatgatcatcaactatacccctttgataggcttgttaatgaacttgaacttgctagaagaacagaccatcagcctctttttgatgttatgctcttgcttcaagatacggttgaagagacaatgcaagcacacgattttacgcttaaagcaagagggcttgagagctctaattcacattttgatatgacattttttatcagtcaaatgggtggtaatattcgtcttgacattgagtacaacacctctttatttacgaaagaaagtgccctacgttttgctcggcactatgaaaattatcttaaaaatgcgctgcgtcaacccagcactatgctcttggatatacgttgtataacccctgaagaaacgaacttcttagcgcaggtttcaaaagggttgcgcagaaatcttcctcaaaaaagctttatccactgttttcttgaacaagcccagaagtttccttacaaagaggcagtggtaacgcctttgggtcggtttagctatgcagatattgcgcaaaaagccattgccatcgctgctgatattcaaagcgctggtatcgaaaaaggtgattttgtaggtcttacatgtaaaagggatgaaaacttagtagcaggaatgcttgggattatgatgagtggtgcaagttatgtttttatgtctgctgaactgcctgaaaaaaggtttgctaaaattgtaaaaactgcccatattaaaagggtatatgcatccacggaagttccctttgttattcaaaatatcacacgtaggatcgatgatgtagcgcctgtggcgtttgtgccagattctttttctacgatggcatattgcatttttacttctggcacaacaggggagccaaagggagtgctcatcacaagggaaaacctagaaaaccttgtttttgcaaccgatatagaaatctacaaacaaagcgataaaactttacgtgaattgtgtgctgtctcaggtgcttttgacgtttcgattaagcaagtttgtgcggccctttcttgcggacacaccctttgtatgccagacgatacaacgctgtatgacccttttttacttcttgagttcattaaaaaagagcagattcatctgctggatatatcgccctctcttttactgatgcttatggaagtagggctttgtgaaacgccattaccctatgttaaaaaactgctcataggctctgaagctgttcagttttctgtgataaaacgatttatgcatgcccatccaaacgtagaggttttcaattgttatggtcctagtgaatgcaccgttgagtctgtgagactaaagatagaggcaacacgagaataccctcatattctccccataggtcacccctgccttaacagcaatgcttatgtgcttgatacgcatatgaacctttgttgcgaaggtgtttatggggaaatccatcttggtggggcatgtgtgggggcaggatacagtaacgagacaaaaaatcggtttacatattttgagaatgaacgtgtctatgcaacaggcgatataggtcgctataaccgttttggcgagatagaaattgcggggagaaaagatactcagcttaaaataagagggtatcgtattgagccagaggagatagaacattgcattcttttagcaccaggtgttgccttggctagtattgcagttttcaaacaaggcactttggatgagatggcagccttttacacaggaacagcagacccagaagaggttaaaaaacatgtcgcttactttgtgccttcttatgccttgcctgcagtctttaagaaaatagataaaatgccactcagtgcaggtggcaaaatagataaaaaggcactcttagcagagcttgtctttaacgaaaaacaaccagttcaacccgaaggacttcatctcgaaattgcatccttatggcacgaccttctgggtcattctggttttggcatggaagatacattctttgatgctggaggaaattctgttttgctggttcaactttacagtaggctgaataaggcctatccagatgtttttaccctagcaggtcttttttccaaaagcagtattgaagctcaagccgaagccattaaagtatattctatgacagatactttgatagagggaatcacactcccctctcatgcccttagtcgtgaggcaaaaggcaatacaaggctttcatcggcattacatgtaacgttgagtattcctcaggcatcggcttatgcgatgtaccttatgttcgagctttatggcatagaagacggggtgagtgctgttattaaaaaaggtatttgctatctttgccgtgtcaatcttcgtggggttgaagatatagatgcgcttataaaaagatgtgcggacaatttagaggattcaaaaagcatacgaccacgagaaggatgtatccccttagtgttatgtgatagtgaaattttagcagaggagtacgtcgctcaaaatggtattgtttttgtgatgcaaaacggttttgttgaggggtatttttctgaaaaaatagacagcaatgaagctgttaagataatggagacgtttatcatgattatacaggcagcctcatgaaaaaaaaacttgaccttaaaaatgccataaagccagatattgaaatcaaaagcgaagctttaaaaggagatgtggctgttgtgggtattggtatacgtgtaggagacatcgtggggccagaagatttttttgatgcgctgctgcgtcaaagagaatttgttaaaaaaccttcatggcagaggatgaaagacacagaagtgtttgctgagacatcttctcaagaatttgcagagcttgcttacattgaagaacacgataggtttgacaacagatttttcaaaattccaccgcacaaagctgagcttatggatccacaagagaagattctgcttcagtgtgctgttgaagcggcagaagatgctggcttagggggtgatgctcttagtggaacaaaaaccgttgtgtttgctggtggcatgggaagcttttttgtgtatgagaatgctcttaaagacatgagcgatgtcaacacacttcttgaactcttaaccccttccatgagtgccgctagaattgctcattttatgaatcttaaaggtggcgcaacggttgttgacacggcttgttcctcttcccttgccgctattttttatgcttatcgtgctgttgcttcgggtgaatattcggctgcttttgctgttgcttcaaagctcattcttaaaccctttgcgtattcaggcgtagagatagcctctaaagatggacgtacaagagcattcgataaagaggctagcggaacgggaggcggagaagctgcggcggctgttgtccttaaatctttggagcgagcactgagcgatggggatgacatttatgccgttatcaaagcaggatcattgaatcataatggtttttctgtaaacattacagcccctgatgcaaaggcgcaaacagaactcatcacgtcagcatggcgcaatgcgcagataaagcctgaggaaattaattttatagaagcccatggaacagcaacaaaaataggcgacgccatagagatagaagcattgaatgcagcctttagaagatacactgacaaaaaagcattttgtacggttggctctgtcaaatcaaacgttggtcatacggataatgccgcaggaatcgtggggtttattaaagccgttctttctgttaaaaatagaatctatcctgcgactgttcactttaatgagcctcatccagcagttgattttaatggctcacccgtctacgctacaggggtaaatgttccactccaaagtcccatcattacagcaggcgttagtgcatttgggcttagtggaaccaatgtgcatatggtgcttcagaatgcaccacaaaaaaaacaattacccagaccgcttcccccttatatttttgcgctttcggcaaaaacagaaatatcccttgatatgcttatccaaaaatgggcaaaatcaaagcttaatgcttatcatcctgctgatatttcagcaacgcttcttcatggaagagggcactatgaagtgcgtgtggcttttgtttttaatacatctgaagaactcttagaaaaacttcaaaagcttactactaaagagccgtgcgatgtttttcaaggggtacacagtattgtcaaggatgtgcgcaatcttaaagaaggtgcgctgagcgttgaaaaagccaaagaaatagggcatgcaatgaaaaacgccttgaagcaaggtgattttgagtccgcactcgcactgtatgtaaaaggggcagaaccatcgtttgatggtgtttttaaagatgcttatacatcaaagttacacttgcctgtctatgcttttgaagctactcgtagatggttttctcatcaaaaggaggtacgttttagtctttcaggtacggatagggttacaacaggtgtttttacggtttttagatcgtttatctcttttacatcagattcactggtgggtgaacatagcgttggtgataaatgcgttatgcctgctagtgggattatagatcaaatttttgatgctgcagaaaagcactatggtcatttggagtatcgcattgaagcattaaagcttcttacttttttggagatacccaaagagggtagctccgaggtggttattcatgtctcaacgacccaagacgctgaagagtgttcgcttatgcagaacatagggggtagctggaaagtgtgtgctcagtggaggatgcataagggaaaatctttgcctttaaaatccaaagaaacagatattgaaaacagataccctgaaaaatatttctcaaaagagagatttgcaaaccaaatgggtggtgttgttgtaagcgataaatggaactgcattgagtatgcaggcaaaaggggtgagtcagcagcttctattttacaagttcctagggatgataggagcttttcaggagcctttttattttacccgcctatggcagatgccgcactgaattttcatcctgaggctaaaaatggtatccccgcatcgtttggtcgaatagagttttttaagcctttgggtacagcatgtctctcttttgccaaacttaaagaaaagagcgataagtatgtggtcttgagcattgttattacggataacaatggtgaaccatgcgttgtgattgatgattatacgctaagactttctacgagtgaggggtatctgcacaaaaaagtatggaattctttagggaagattgctccaaaagtgttcgaagatgtttttgtatttgaggaaagtgatgctgaaaagcccctctttcaagaaagactcaagcaaatttattcagtagcatctgtacgcattgtatacaaagtaaatactctcaacaagcctgaaaaaacattgcgtactctttttgactttttgtgctctctttcaaaaatggaaggtaagatagagcttattatctcagcagatgagaggacacacttgcttcatggtgctatggcggcgatgggtcgaacctttgaatttgaacatccttgtgcaagattccgatttgttgaaacgtatgaagagagcattaatctttttgatgtgtttcattttcaaggatttagatttagggtttctaatgggactctccagaggttggagattgaagagtgtggtatagggcaagggttggagttaaaaaacggtggtcaatatcttattacagggggacttggtggattaggtctttttactgcagaatatgtgtctaaaaaagttgattgtgcattcgttttgatgtcccgtagtggatttgcaccacaagatgagtggaattatctctctgagttaaaaggcatatctgcggatattatagaaaagatacaaaaagtaaaaaacaaccattgttcagtgacaatttttaaaggagatgtctctttggagcgtgatcttgcacgccttaaagaacaatttggtcattttgatgggatttttcatgccgcaggttctgctacagagaggttttttatcaaaaatgatttaaacgcttttttaagaacagtgttaccaaaggtcagaggaacacttaacgtgcttgagatgtttcaagatactggtttttttctgctcttctcatcgctttctggcttgacatcggcacctggtcaagcaggctattgtgctgcaaatgcttttatggattctttggcagaagatatgcctgaaaaaattctttccgtgggttggccacgatggagtgaagttggcatggcaattggacttactagcaagggggatgattatgaagctataaacttaaaagaagcatctaggatactggatattcttcctgcggggggatatgttgctgttggcaaaagtcatgcgccactgagacctttactagaagaagcctcagtgttaaaaagctcactcgttgcttctgcaaaaacgaagatgagtaagagcgttgaactaacaggtgcagaatcatttacgcacatgcaaatagatattggtactatctgggcgggtgaacttggatatgaaacaattgatatttatgaggattacgaagatttaggtggagattcgattgcatctctttccattcatgaaaaattagaaaagagcttaggcataagtctttctttttcggagttgtttgaaaataaaacaattgccactatggctcaataccttgagcaaaaaattcaccaaaagaataagccaacaaaggaggataatatccttgttttgaggcaaaaaggtgaacgaaagattatctgttttccaccagggacagttatgggaaatgtttacgctggggtagcaacggctttgagtggttatgcggtttatgctttgaattatattcagtcgaagaatcccccagagttgtttgcagatatgacagagtccgttcagccagaaggtgaactgattttactagggtattctgtggggggcaatttagcctatgaaacagcgaaggttcttgaaaaaagaggcagaaggattagagcacttatctttttggacaactttcgacgacttgagctttttgaacaaagtgatgaagcataccaaaggggagcaagggagtatttgagcattcttgaaaaaacacaaggaagcgacttagataaagaggagattctagcaagaattgaacactacgacagattcattgattcaagacgtgaacaagatcttattagtgctccaatttatcttataaaggctgaaaaaagtgctgccaatgccccaatgaaaatctcacaagatgcatggggtgagcttacaacgcattttgaaatctttcaaggagatggaaaacacatggagatgctttttggtgaatacttcgaaaaaaatattgagattataagaagtattttaaacacattaaagtaattgctatttatcttactataaatagcaattacactcttaaaggagacgaagatggcaacagaagtaaaagaagaaaaagtggtagaaggatccgtttttgaaacggtaagtttcgatgagagaaaacaaaaaactgacgagattgtcaagcgtcatgtgtacggtgctatgggtgtagggcttgtacctattcctctcatagattttattggtgtgacaagtgttcaagtggatatgctctcaacattggcgcaatattatggtgtgacattcaaaaaagagatggcaaaaaatctgataggatctcttgtcggtggagctgttcctgctggattgggaatgcccattgctagtattcttaagggcataccacttgtaggtcaaacggtgggcgctcttggaatgtctatggttgctggtgcttcaacgtatgccgtaggtagagtgtttgtccaacattttgaatcaggtggaacatttttaagttttgatcctggtgcggtaagagagtattttaaagaagagtttaatataggcaaaacagtcgcaaaagaagcctctgcaaagaaataacgatgagcttatttctttatatttttctttgccttgcaataggggtatttggtctgaacaagaagttgggattttggggctatttttttgcatcacttttgctgacacctcttcttgggattatcttgctccttgcatcaggagagaagaaaagcagaaccgattaagtcatgcaccttgaaactgtcttactacggtattgcctttttgatgcgtatggcgaagctaacccgtgggtgtctcttatgcctgaatgcaacaggaaaagatgcacccactttaggaaggacgaatcaaaaataaatcatgcagctgttaggctcttacttgcatgtgcattgggcaaaaaaagagacattattgctaagattagaaaaacggatgcaggaaagccttttatagataaaatgcctttttttagcttgagtcatacaacaaaagctgtagcggtaagtacatgtaaaaatcagcccacaggtgttgatgcagagttattaaggcacattgatgttaatgattttagccactatttatcaagggaagagaaggataagattaagggttcatctgaggagtttttaaggctgtggtgtacgaaagaagctgtttttaaagcagctggaactggactgcgtgaaccttttacggatgtggttgttatgccagattttgctctttttgatcataacacatggtactataaaaccttgattgtggagagtgtggtaatgagtgtttgttgtgcaagaccttttaagattgatatacaagagcttaaaccgtgcgaattacgggctttagattaatgtggatccgaattcgagctccgtcgacaagcttgcggccgcactcgagagcgcttggagccacccgcagttcgaaaaataatgagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca